(19) United States
(12) Patent Application Publication
(10) Pub. No.: US 2003/0139572 A1
(43) Pub. Date: Jul. 24, 2003

(54) Novel compounds

(76) Inventors: Panjak Agarwal, King of Prussia, PA (US); Karen S. Kabnick, Lafayette Hill, PA (US); Ying-Ta Lai, Upper Darby, PA (US); Paul R. Murdock, Harlow Essex (GB); Safia K. Rizvi, Philadelphia, PA (US); Randall F. Smith, Lafayette Hill, PA (US); Zhaoying Xiang, Fort Lee, NJ (US)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/239,663

(22) PCT Filed: Mar. 22, 2001
(86) PCT No.: PCT/US01/09226

(51) Int. Cl. C12P 21/02; C12N 5/06; C07K 14/47; C07H 21/04

(52) U.S. Cl. 530/350; 435/69.1; 435/320.1; 435/325; 536/23.5

(57) ABSTRACT

Polypeptides and polynucleotides of the genes set forth in Table 1 and methods for producing such polypeptides by recombinant techniques are disclosed. Also disclosed are methods for utilizing polypeptides and polynucleotides of the genes set forth in Table 1 in diagnostic assays.

### FIELD OF INVENTION

This invention relates to newly identified polypeptides and polynucleotides encoding such polypeptides, to their use in diagnosis and in identifying compounds that may be agonists, antagonists that are potentially useful in therapy, and to production of such polypeptides and polynucleotides. The polynucleotides and polypeptides of the present invention also relate to proteins with signal sequences which allow them to be secreted extracellularly or membrane-associated (hereinafter often referred collectively as secreted proteins or secreted polypeptides).

### BACKGROUND OF THE INVENTION

The drug discovery process is currently undergoing a fundamental revolution as it embraces “functional genomics”, that is, high throughput genome- or gene-based biology. This approach as a means to identify genes and gene products as therapeutic targets is rapidly superseding earlier approaches based on “positional cloning”. A phenotype, that is a biological function or genetic disease, would be identified and this would then be tracked back to the responsible gene, based on its genetic map position.

Functional genomics relies heavily on high-throughput DNA sequencing technologies and the various tools of bioinformatics to identify gene sequences of potential interest from the many molecular biology databases now available. There is a continuing need to identify and characterise further genes and their related polypeptides/proteins, as targets for drug discovery.

Proteins and polypeptides that are naturally secreted into blood, lymph and other body fluids, or secreted into the cellular membrane are of primary interest for pharmaceutical research and development. The reason for this interest is the relative ease to target protein therapeutics into their place of action (body fluids or the cellular membrane). The natural pathway for protein secretion into extracellular space is the endoplasmic reticulum in eukaryotes and the inner membrane in prokaryotes (Palade, 1975, Science, 189, 347; Milstein, Brownlee, Harrison, and Mathews, 1972, Nature New Biol., 239, 117; Blobel, and Dobberstein, 1975, J. Cell. Biol., 67, 835). On the other hand, there is no known natural pathway for exporting a protein from the exterior of the cells into the cytosol (with the exception of pinocytosis, a mechanism of snake venom toxin intrusion into cells). Therefore targeting protein therapeutics into cells poses extreme difficulties.

The secreted and membrane-associated proteins include but are not limited to all peptide hormones and their receptors (including but not limited to insulin, growth hormones, chemokines, cytokines, neuropeptides, integrins, kallikreins, lamins, melanins, natriuretic hormones, neuropsin, neurotropins, pituitiary hormones, pleiotropins, prostaglandins, secretogranins, selecting, thromboglobulins, thymosins), the breast and colon cancer gene products, leptin, the obesity gene protein and its receptors, serum albumin, superoxide dismutase, spliceosome proteins, 7TM (transmembrane) proteins also called as G-protein coupled receptors, immunoglobulins, several families of serine proteinases (including but not limited to proteins of the blood coagulation cascade, digestive enzymes), deoxyribonuclease I, etc.

Therapeutics based on secreted or membrane-associated proteins approved by FDA or foreign agencies include but are not limited to insulin, glucagon, growth hormone, chorionic gonadotropin, follicle stimulating hormone, luteinizing hormone, calcitonin, adrenocorticotropic hormone (ACTH), vasopressin, interleukines, interferones, immunoglobulins, lactoferrin (diverse products marketed by several companies), tissue-type plasminogen activator (Alteplase by Genentech), hyaulorindase (Wydase by Wyeth-Ayerst), dornase alpha (Pulmozyme\ by Genentech), Chymodiactin (chymopapain by Knoll), alglucerase (Ceredase by Genzyme), streptokinase (Kabikinase by Pharmacia) (Streptase by Astra), etc. This indicates that secreted and membrane-associated proteins have an established, proven history as therapeutic targets. Clearly, there is a need for identification and characterization of further secreted and membrane-associated proteins which can play a role in preventing, ameliorating or correcting dysfunction or disease, including but not limited to diabetes, breast-, prostate-, colon cancer and other malignant tumors, hyper- and hypotension, obesity, bulimia, anorexia, growth abnormalities, asthma, manic depression, dementia, delirium, mental retardation, Huntington's disease, Tourette's syndrome, schizophrenia, growth, mental or sexual development disorders, and dysfunctions of the blood cascade system including those leading to stroke. The proteins, of the present invention which include the signal sequences are also useful to further elucidate the mechanism of protein transport which at present is not entirely understood, and thus can be used as research tools.

### SUMMARY OF THE INVENTION

The present invention relates to particular polypeptides and polynucleotides of the genes set forth in Table I including recombinant materials and methods for their production. Such polypeptides and polynucleotides are of interest in relation to methods of treatment of certain diseases, including, but not limited to, the diseases set forth in Tables III and V, hereinafter referred to as “diseases of the invention”. In a further aspect, the invention relates to methods for identifying agonists and antagonists (e.g., inhibitors) using the materials provided by the invention, and treating conditions associated with imbalance of polypeptides and/or polynucleotides of the genes set forth in Table I with the identified compounds. In still a further aspect, the invention relates to diagnostic assays for detecting diseases associated with inappropriate activity or levels the genes set forth in Table I. Another aspect of the invention concerns a polynucleotide comprising any of the nucleotide sequences set forth in the Sequence Listing and a polypeptide comprising a polypeptide encoded by the nucleotide sequence. In another aspect, the invention relates to a polypeptide comprising any of the polypeptide sequences set forth in the Sequence Listing and recombinant materials and methods for their production. Another aspect of the invention relates to methods for using such polypeptides and polynucleotides. Such uses include the treatment of diseases, abnormalities and disorders (hereinafter simply referred to as diseases) caused by abnormal expression, production, function and or metabolism of the genes of this invention, and such diseases are readily apparent by those skilled in the art from the homology to other proteins disclosed for each attached sequence. In still another aspect, the invention relates to methods to identify agonists and antagonists using the materials provided by the invention, and treating conditions associated with the imbalance with the identified compounds. Yet another aspect of the invention relates to diagnostic assays for detecting diseases associated with inappropriate activity or levels of the secreted proteins of the present invention.

### DESCRIPTION OF THE INVENTION

In a first aspect, the present invention relates to polypeptides the genes set forth in Table I. Such polypeptides include:

(a) an isolated polypeptide encoded by a polynucleotide comprising a sequence set forth in the Sequence Listing, herein when referring to polynucleotides or polypeptides of the Sequence Listing, a reference is also made to the Sequence Listing referred to in the Sequence Listing;

(b) an isolated polypeptide comprising a polypeptide sequence having at least 95%, 96%, 97%, 98%, or 99% identity to a polypeptide sequence set forth in the Sequence Listing;

(c) an isolated polypeptide comprising a polypeptide sequence set forth in the Sequence Listing;

(d) an isolated polypeptide having at least 95%, 96%, 97%, 98%, or 99% identity to a polypeptide sequence set forth in the Sequence Listing;

(e) a polypeptide sequence set forth in the Sequence Listing; and

(f) an isolated polypeptide having or comprising a polypeptide sequence that has an Identity Index of 0.95, 0.96, 0.97, 0.98, or 0.99 compared to a polypeptide sequence set forth in the Sequence Listing;

(g) fragments and variants of such polypeptides in (a) to (f).

Polypeptides of the present invention are believed to be members of the gene families set forth in Table II. They are therefore of therapeutic and diagnostic interest for the reasons set forth in Tables III and V. The biological properties of the polypeptides and polynucleotides of the genes set forth in Table I are hereinafter referred to as “the biological activity” of polypeptides and polynucleotides of the genes set forth in Table I. Preferably, a polypeptide of the present invention exhibits at least one biological activity of the genes set forth in Table I.

Polypeptides of the present invention also include variants of the aforementioned polypeptides, including all allelic forms and splice variants. Such polypeptides vary from the reference polypeptide by insertions, deletions, and substitutions that may be conservative or non-conservative, or any combination thereof. Particularly preferred variants are those in which several, for instance from 50 to 30, from 30 to 20, from 20 to 10, from 10 to 5, from 5 to 3, from 3 to 2, from 2 to 1 or 1 amino acids are inserted, substituted, or deleted, in any combination.

Preferred fragments of polypeptides of the present invention include an isolated polypeptide comprising an amino acid sequence having at least 30, 50 or 100 contiguous amino acids from an amino acid sequence set forth in the Sequence Listing, or an isolated polypeptide comprising an amino acid sequence having at least 30, 50 or 100 contiguous amino acids truncated or deleted from an amino acid sequence set forth in the Sequence Listing. Preferred fragments are biologically active fragments that mediate the biological activity of polypeptides and polynucleotides of the genes set forth in Table I, including those with a similar activity or an improved activity, or with a decreased undesirable activity. Also preferred are those fragments that are antigenic or immunogenic in an animal, especially in a human.

Fragments of a polypeptide of the invention may be employed for producing the corresponding full-length polypeptide by peptide synthesis; therefore, these variants may be employed as intermediates for producing the full-length polypeptides of the invention. A polypeptide of the present invention may be in the form of the “mature” protein or may be a part of a larger protein such as a precursor or a fusion protein. It is often advantageous to include an additional amino acid sequence that contains secretory or leader sequences, pro-sequences, sequences that aid in purification, for instance multiple histidine residues, or an additional sequence for stability during recombinant production.

Polypeptides of the present invention can be prepared in any suitable manner, for instance by isolation form naturally occurring sources, from genetically engineered host cells comprising expression systems (vide infra) or by chemical synthesis, using for instance automated peptide synthesizers, or a combination of such methods. Means for preparing such polypeptides are well understood in the art.

In a further aspect, the present invention relates to polynucleotides of the genes set forth in Table I. Such polynucleotides include:

(a) an isolated polynucleotide comprising a polynucleotide sequence having at least 95%, 96%, 97%, 98%, or 99% identity to a polynucleotide sequence set forth in the Sequence Listing;

(b) an isolated polynucleotide comprising a polynucleotide set forth in the Sequence Listing;

(c) an isolated polynucleotide having at least 95%, 96%, 97%, 98%, or 99% identity to a polynucleotide set forth in the Sequence Listing;

(d) an isolated polynucleotide set forth in the Sequence Listing;

(e) an isolated polynucleotide comprising a polynucleotide sequence encoding a polypeptide sequence having at least 95%, 96%, 97%, 98%, or 99% identity to a polypeptide sequence set forth in the Sequence Listing;

(f) an isolated polynucleotide comprising a polynucleotide sequence encoding a polypeptide set forth in the Sequence Listing;

(g) an isolated polynucleotide having a polynucleotide sequence encoding a polypeptide sequence having at least 95%, 96%, 97%, 98%, or 99% identity to a polypeptide sequence set forth in the Sequence Listing;

(h) an isolated polynucleotide encoding a polypeptide set forth in the Sequence Listing;

(i) an isolated polynucleotide having or comprising a polynucleotide sequence that has an Identity Index of 0.95, 0.96, 0.97, 0.98, or 0.99 compared to a polynucleotide sequence set forth in the Sequence Listing;

(j) an isolated polynucleotide having or comprising a polynucleotide sequence encoding a polypeptide sequence that has an Identity Index of 0.95, 0.96, 0.97, 0.98, or 0.99 compared to a polypeptide sequence set forth in the Sequence Listing; and

polynucleotides that are fragments and variants of the above mentioned polynucleotides or that are complementary to above mentioned polynucleotides, over the entire length thereof.

Preferred fragments of polynucleotides of the present invention include an isolated polynucleotide comprising an nucleotide sequence having at least 15, 30, 50 or 100 contiguous nucleotides from a sequence set forth in the Sequence Listing, or an isolated polynucleotide comprising a sequence having at least 30, 50 or 100 contiguous nucleotides truncated or deleted from a sequence set forth in the Sequence Listing.

Preferred variants of polynucleotides of the present invention include splice variants, allelic variants, and polymorphisms, including polynucleotides having one or more single nucleotide polymorphisms (SNPs).

Polynucleotides of the present invention also include polynucleotides encoding polypeptide variants that comprise an amino acid sequence set forth in the Sequence Listing and in which several, for instance from 50 to 30, from 30 to 20, from 20 to 10, from 10 to 5, from 5 to 3, from 3 to 2, from 2 to 1 or 1 amino acid residues are substituted, deleted or added, in any combination.

In a further aspect, the present invention provides polynucleotides that are RNA transcripts of the DNA sequences of the present invention. Accordingly, there is provided an RNA polynucleotide that:

(a) comprises an RNA transcript of the DNA sequence encoding a polypeptide set forth in the Sequence Listing;

(b) is a RNA transcript of a DNA sequence encoding a polypeptide set forth in the Sequence Listing;

(c) comprises an RNA transcript of a DNA sequence set forth in the Sequence Listing; or

(d) is a RNA transcript of a DNA sequence set forth in the Sequence Listing;

and RNA polynucleotides that are complementary thereto.

The polynucleotide sequences set forth in the Sequence Listing show homology with the polynucleotide sequences set forth in Table II. A polynucleotide sequence set forth in the Sequence Listing is a cDNA sequence that encodes a polypeptide set forth in the Sequence Listing. A polynucleotide sequence encoding a polypeptide set forth in the Sequence Listing may be identical to a polypeptide encoding a sequence set forth in the Sequence Listing or it may be a sequence other than a sequence set forth in the Sequence Listing, which, as a result of the redundancy (degeneracy) of the genetic code, also encodes a polypeptide set forth in the Sequence Listing. A polypeptide of a sequence set forth in the Sequence Listingis related to other proteins of the gene families set forth in Table II, having homology and/or structural similarity with the polypeptides set forth in Table II. Preferred polypeptides and polynucleotides of the present invention are expected to have, inter alia, similar biological functions/properties to their homologous polypeptides and polynucleotides. Furthermore, preferred polypeptides and polynucleotides of the present invention have at least one activity of the genes set forth in Table I.

Polynucleotides of the present invention may be obtained using standard cloning and screening techniques from a cDNA library derived from mRNA from the tissues set forth in Table IV (see for instance, Sambrook et al., Molecular Cloning: A Laboratory Manual, 2nd Ed., Cold Spring Harbor Laboratory Press, Cold Spring Harbor, N.Y. (1989)). Polynucleotides of the invention can also be obtained from natural sources such as genomic DNA libraries or can be synthesized using well known and commercially available techniques.

When polynucleotides of the present invention are used for the recombinant production of polypeptides of the present invention, the polynucleotide may include the coding sequence for the mature polypeptide, by itself, or the coding sequence for the mature polypeptide in reading frame with other coding sequences, such as those encoding a leader or secretory sequence, a pre-, or pro- or prepro-protein sequence, or other fusion peptide portions. For example, a marker sequence that facilitates purification of the fused polypeptide can be encoded. In certain preferred embodiments of this aspect of the invention, the marker sequence is a hexa-histidine peptide, as provided in the pQE vector (Qiagen, Inc.) and described in Gentz et al., Proc Natl Acad Sci USA (1989) 86:821-824, or is an HA tag. A polynucleotide may also contain non-coding 5′ and 3′ sequences, such as transcribed, non-translated sequences, splicing and polyadenylation signals, ribosome binding sites and sequences that stabilize mRNA.

Polynucleotides that are identical, or have sufficient identity to a polynucleotide sequence set forth in the Sequence Listing, may be used as hybridization probes for cDNA and genomic DNA or as primers for a nucleic acid amplification reaction (for instance, PCR). Such probes and primers may be used to isolate full-length cDNAs and genomic clones encoding polypeptides of the present invention and to isolate cDNA and genomic clones of other genes (including genes encoding paralogs from human sources and orthologs and paralogs from species other than ) that have a high sequence similarity to sequences set forth in the Sequence Listing, typically at least 95% identity. Preferred probes and primers will generally comprise at least 15 nucleotides, preferably, at least 30 nucleotides and may have at least 50, if not at least 100 nucleotides. Particularly preferred probes will have between 30 and 50 nucleotides. Particularly preferred primers will have between 20 and 25 nucleotides.

A polynucleotide encoding a polypeptide of the present invention, including homologs from species other than, may be obtained by a process comprising the steps of screening a library under stringent hybridization conditions with a labeled probe having a sequence set forth in the Sequence Listing or a fragment thereof, preferably of at least 15 nucleotides; and isolating full-length cDNA and genomic clones containing the polynucleotide sequence set forth in the Sequence Listing. Such hybridization techniques are well known to the skilled artisan. Preferred stringent hybridization conditions include overnight incubation at 42° C. in a solution comprising: 50% formamide, 5×SSC (150 mM NaCl, 15 mnM trisodium citrate), 50 mM sodium phosphate (pH 7.6), 5×Denhardt's solution, 10% dextran sulfate, and 20 microgram/ml denatured, sheared salmon sperm DNA; followed by washing the filters in 0.1×SSC at about 65° C. Thus the present invention also includes isolated polynucleotides, preferably with a nucleotide sequence of at least 100, obtained by screening a library under stringent hybridization conditions with a labeled probe having the sequence set forth in the Sequence Listing or a fragment thereof, preferably of at least 15 nucleotides.

The skilled artisan will appreciate that, in many cases, an isolated cDNA sequence will be incomplete, in that the region coding for the polypeptide does not extend all the way through to the 5′terminus. This is a consequence of reverse transcriptase, an enzyme with inherently low “processivity” (a measure of the ability of the enzyme to remain attached to the template during the polymerisation reaction), failing to complete a DNA copy of the mRNA template during first strand cDNA synthesis.

There are several methods available and well known to those skilled in the art to obtain full-length cDNAs, or extend short cDNAs, for example those based on the method of Rapid Amplification of cDNA ends (RACE) (see, for example, Frohman et al., Proc Nat Acad Sci USA 85, 8998-9002, 1988). Recent modifications of the technique, exemplified by the Marathon (trade mark) technology (Clontech Laboratories Inc.) for example, have significantly simplified the search for longer cDNAs. In the Marathon (trade mark) technology, cDNAs have been prepared from mRNA extracted from a chosen tissue and an ‘adaptor’ sequence ligated onto each end. Nucleic acid amplification (PCR) is then carried out to amplify the “missing” 5′ end of the cDNA using a combination of gene specific and adaptor specific oligonucleotide primers. The PCR reaction is then repeated using ‘nested’ primers, that is, primers designed to anneal within the amplified product (typically an adapter specific primer that anneals further 3′ in the adaptor sequence and a gene specific primer that anneals further 5′ in the known gene sequence). The products of this reaction can then be analyzed by DNA sequencing and a full-length cDNA constructed either by joining the product directly to the existing cDNA to give a complete sequence, or carrying out a separate full-length PCR using the new sequence information for the design of the 5′ primer.

Recombinant polypeptides of the present invention may be prepared by processes well known in the art from genetically engineered host cells comprising expression systems. Accordingly, in a further aspect, the present invention relates to expression systems comprising a polynucleotide or polynucleotides of the present invention, to host cells which are genetically engineered with such expression systems and to the production of polypeptides of the invention by recombinant techniques. Cell-free translation systems can also be employed to produce such proteins using RNAs derived from the DNA constructs of the present invention.

For recombinant production, host cells can be genetically engineered to incorporate expression systems or portions thereof for polynucleotides of the present invention. Polynucleotides may be introduced into host cells by methods described in many standard laboratory manuals, such as Davis et al., Basic Methods in Molecular Biology (1986) and Sambrook et al.(ibid). Preferred methods of introducing polynucleotides into host cells include, for instance, calcium phosphate transfection, DEAE-dextran mediated transfection, transvection, micro-injection, cationic lipid-mediated transfection, electroporation, transduction, scrape loading, ballistic introduction or infection.

Representative examples of appropriate hosts include bacterial cells, such as Streptococci, Staphylococci, E. coli, Streptomyces and Bacillus subtilis cells; fungal cells, such as yeast cells and Aspergillus cells; insect cells such as Drosophila S2 and Spodoptera Sf9 cells; animal cells such as CHO, COS, HeLa, C127, 3T3, BHK, HEK 293 and Bowes melanoma cells; and plant cells.

A great variety of expression systems can be used, for instance, chromosomal, episomal and virus-derived systems, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. The expression systems may contain control regions that regulate as well as engender expression. Generally, any system or vector that is able to maintain, propagate or express a polynucleotide to produce a polypeptide in a host may be used. The appropriate polynucleotide sequence may be inserted into an expression system by any of a variety of well-known and routine techniques, such as, for example, those set forth in Sambrook et al., (ibid). Appropriate secretion signals may be incorporated into the desired polypeptide to allow secretion of the translated protein into the lumen of the endoplasmic reticulum, the periplasmic space or the extracellular environment. These signals may be endogenous to the polypeptide or they may be heterologous signals.

If a polypeptide of the present invention is to be expressed for use in screening assays, it is generally preferred that the polypeptide be produced at the surface of the cell. In this event, the cells may be harvested prior to use in the screening assay. If the polypeptide is secreted into the medium, the medium can be recovered in order to recover and purify the polypeptide. If produced intracellularly, the cells must first be lysed before the polypeptide is recovered.

Polypeptides of the present invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography is employed for purification. Well known techniques for refolding proteins may be employed to regenerate active conformation when the polypeptide is denatured during intracellular synthesis, isolation and/or purification.

Polynucleotides of the present invention may be used as diagnostic reagents, through detecting mutations in the associated gene. Detection of a mutated form of a gene is characterized by the polynucleotides set forth in the Sequence Listing in the cDNA or genomic sequence and which is associated with a dysfunction. Will provide a diagnostic tool that can add to, or define, a diagnosis of a disease, or susceptibility to a disease, which results from under-expression, over-expression or altered spatial or temporal expression of the gene. Individuals carrying mutations in the gene may be detected at the DNA level by a variety of techniques well known in the art.

Nucleic acids for diagnosis may be obtained from a subject's cells, such as from blood, urine, saliva, tissue biopsy or autopsy material. The genomic DNA may be used directly for detection or it may be amplified enzymatically by using PCR, preferably RT-PCR, or other amplification techniques prior to analysis. RNA or cDNA may also be used in similar fashion. Deletions and insertions can be detected by a change in size of the amplified product in comparison to the normal genotype. Point mutations can be identified by hybridizing amplified DNA to labeled nucleotide sequences of the genes set forth in Table I. Perfectly matched sequences can be distinguished from mismatched duplexes by RNase digestion or by differences in melting temperatures. DNA sequence difference may also be detected by alterations in the electrophoretic mobility of DNA fragments in gels, with or without denaturing agents, or by direct DNA sequencing (see, for instance, Myers et al., Science (1985) 230:1242). Sequence changes at specific locations may also be revealed by nuclease protection assays, such as RNase and S1 protection or the chemical cleavage method (see Cotton et al., Proc Natl Acad Sci USA (1985) 85: 4397-4401).

An array of oligonucleotides probes comprising polynucleotide sequences or fragments thereof of the genes set forth in Table I can be constructed to conduct efficient screening of e.g., genetic mutations. Such arrays are preferably high density arrays or grids. Array technology methods are well known and have general applicability and can be used to address a variety of questions in molecular genetics including gene expression, genetic linkage, and genetic variability, see, for example, M. Chee et al., Science, 274, 610-613 (1996) and other references cited therein.

Detection of abnormally decreased or increased levels of polypeptide or mRNA expression may also be used for diagnosing or determining susceptibility of a subject to a disease of the invention. Decreased or increased expression can be measured at the RNA level using any of the methods well known in the art for the quantitation of polynucleotides, such as, for example, nucleic acid amplification, for instance PCR, RT-PCR, RNase protection, Northern blotting and other hybridization methods. Assay techniques that can be used to determine levels of a protein, such as a polypeptide of the present invention, in a sample derived from a host are well-known to those of skill in the art. Such assay methods include radio-immunoassays, competitive-binding assays, Western Blot analysis and ELISA assays.

Thus in another aspect, the present invention relates to a diagnostic kit comprising:

(a) a polynucleotide of the present invention, preferably the nucleotide sequence set forth in the Sequence Listing, or a fragment or an RNA transcript thereof;

(b) a nucleotide sequence complementary to that of (a);

(c) a polypeptide of the present invention, preferably the polypeptide set forth in the Sequence Listing or a fragment thereof; or

(d) an antibody to a polypeptide of the present invention, preferably to the polypeptide set forth in the Sequence Listing.

It will be appreciated that in any such kit, (a), (b), (c) or (d) may comprise a substantial component. Such a kit will be of use in diagnosing a disease or susceptibility to a disease, particularly diseases of the invention, amongst others.

The polynucleotide sequences of the present invention are valuable for chromosome localisation studies. The sequences set forth in the Sequence Listing are specifically targeted to, and can hybridize with, a particular location on an individual human chromosome. The mapping of relevant sequences to chromosomes according to the present invention is an important first step in correlating those sequences with gene associated disease. Once a sequence has been mapped to a precise chromosomal location, the physical position of the sequence on the chromosome can be correlated with genetic map data. Such data are found in, for example, V. McKusick, Mendelian Inheritance in Man (available on-line through Johns Hopkins University Welch Medical Library). The relationship between genes and diseases that have been mapped to the same chromosomal region are then identified through linkage analysis (co-inheritance of physically adjacent genes). Precise human chromosomal localisations for a genomic sequence (gene fragment etc.) can be determined using Radiation Hybrid (RH) Mapping (Walter, M. Spillett, D., Thomas, P., Weissenbach, J., and Goodfellow, P., (1994) A method for constructing radiation hybrid maps of whole genomes, Nature Genetics 7, 22-28). A number of RH panels are available from Research Genetics (Huntsville, Ala., USA) e.g. the GeneBridge4 RH panel (Hum Mol Genet 1996 Mar;5(3):33946 A radiation hybrid map of the human genome. Gyapay G, Schmitt K, Fizames C, Jones H, Vega-Czarny N, Spillett D, Muselet D, Prud'Homme J F, Dib C, Auffray C, Morissette J, Weissenbach J, Goodfellow P N). To determine the chromosomal location of a gene using this panel, 93 PCRs are performed using primers designed from the gene of interest on RH DNAs. Each of these DNAs contains random human genomic fragments maintained in a hamster background (human/hamster hybrid cell lines). These PCRs result in 93 scores indicating the presence or absence of the PCR product of the gene of interest. These scores are compared with scores created using PCR products from genomic sequences of known location. This comparison is conducted at http://www.genome.wi.mit.edu/.

The polynucleotide sequences of the present invention are also valuable tools for tissue expression studies. Such studies allow the determination of expression patterns of polynucleotides of the present invention which may give an indication as to the expression patterns of the encoded polypeptides in tissues, by detecting the mRNAs that encode them. The techniques used are well known in the art and include in situ hydridization techniques to clones arrayed on a grid, such as cDNA microarray hybridization (Schena et al, Science, 270, 467-470, 1995 and Shalon et al, Genome Res, 6, 639-645, 1996) and nucleotide amplification techniques such as PCR. A preferred method uses the TAQMAN (Trade mark) technology available from Perkin Elmer. Results from these studies can provide an indication of the normal function of the polypeptide in the organism. In addition, comparative studies of the normal expression pattern of mRNAs with that of mRNAs encoded by an alternative form of the same gene (for example, one having an alteration in polypeptide coding potential or a regulatory mutation) can provide valuable insights into the role of the polypeptides of the present invention, or that of inappropriate expression thereof in disease. Such inappropriate expression may be of a temporal, spatial or simply quantitative nature.

A further aspect of the present invention relates to antibodies. The polypeptides of the invention or their fragments, or cells expressing them, can be used as immunogens to produce antibodies that are immunospecific for polypeptides of the present invention. The term “immunospecific” means that the antibodies have substantially greater affinity for the polypeptides of the invention than their affinity for other related polypeptides in the prior art.

Antibodies generated against polypeptides of the present invention may be obtained by administering the polypeptides or epitope-bearing fragments, or cells to an animal, preferably a non-human animal, using routine protocols. For preparation of monoclonal antibodies, any technique which provides antibodies produced by continuous cell line cultures can be used. Examples include the hybridoma technique (Kohler, G. and Milstein, C., Nature (1975) 256:495-497), the trioma technique, the human B-cell hybridoma technique (Kozbor et al., Immunology Today (1983) 4:72) and the EBV-hybridoma technique (Cole et al., Monoclonal Antibodies and Cancer Therapy, 77-96, Alan R. Liss, Inc., 1985).

Techniques for the production of single chain antibodies, such as those described in U.S. Pat. No. 4,946,778, can also be adapted to produce single chain antibodies to polypeptides of this invention. Also, transgenic mice, or other organisms, including other mammals, may be used to express humanized antibodies.

The above-described antibodies may be employed to isolate or to identify clones expressing the polypeptide or to purify the polypeptides by affinity chromatography. Antibodies against polypeptides of the present invention may also be employed to treat diseases of the invention, amongst others.

Polypeptides and polynucleotides of the present invention may also be used as vaccines. Accordingly, in a further aspect, the present invention relates to a method for inducing an immunological response in a mammal that comprises inoculating the mammal with a polypeptide of the present invention, adequate to produce antibody and/or T cell immune response, including, for example, cytokine-producing T cells or cytotoxic T cells, to protect said animal from disease, whether that disease is already established within the individual or not. An immunological response in a mammal may also be induced by a method comprises delivering a polypeptide of the present invention via a vector directing expression of the polynucleotide and coding for the polypeptide in vivo in order to induce such an immunological response to produce antibody to protect said animal from diseases of the invention. One way of administering the vector is-by accelerating it into the desired cells as a coating on particles or otherwise. Such nucleic acid vector may comprise DNA, RNA, a modified nucleic acid, or a DNA/RNA hybrid. For use a vaccine, a polypeptide or a nucleic acid vector will be normally provided as a vaccine formulation (composition). The formulation may further comprise a suitable carrier. Since a polypeptide may be broken down in the stomach, it is preferably administered parenterally (for instance, subcutaneous, intra-muscular, intravenous, or intra-dermal injection). Formulations suitable for parenteral administration include aqueous and non-aqueous sterile injection solutions that may contain anti-oxidants, buffers, bacteriostats and solutes that render the formulation instonic with the blood of the recipient; and aqueous and non-aqueous sterile suspensions that may include suspending agents or thickening agents. The formulations may be presented in unit-dose or multi-dose containers, for example, sealed ampoules and vials and may be stored in a freeze-dried condition requiring only the addition of the sterile liquid carrier immediately prior to use. The vaccine formulation may also include adjuvant systems for enhancing the immunogenicity of the formulation, such as oil-in water systems and other systems known in the art. The dosage will depend on the specific activity of the vaccine and can be readily determined by routine experimentation.

Polypeptides of the present invention have one or more biological functions that are of relevance in one or more disease states, in particular the diseases of the invention hereinbefore mentioned. It is therefore useful to identify compounds that stimulate or inhibit the function or level of the polypeptide. Accordingly, in a further aspect, the present invention provides for a method of screening compounds to identify those that stimulate or inhibit the function or level of the polypeptide. Such methods identify agonists or antagonists that may be employed for therapeutic and prophylactic purposes for such diseases of the invention as hereinbefore mentioned. Compounds may be identified from a variety of sources, for example, cells, cell-free preparations, chemical libraries, collections of chemical compounds, and natural product mixtures. Such agonists or antagonists so-identified may be natural or modified substrates, ligands, receptors, enzymes, etc., as the case may be, of the polypeptide; a structural or functional mimetic thereof (see Coligan et al., Current Protocols in Immunology 1(2):Chapter 5 (1991)) or a small molecule. Such small molecules preferably have a molecular weight below 2,000 daltons, more preferably between 300 and 1,000 daltons, and most preferably between 400 and 700 daltons. It is preferred that these small molecules are organic molecules.

The screening method may simply measure the binding of a candidate compound to the polypeptide, or to cells or membranes bearing the polypeptide, or a fusion protein thereof, by means of a label directly or indirectly associated with the candidate compound. Alternatively, the screening method may involve measuring or detecting (qualitatively or quantitatively) the competitive binding of a candidate compound to the polypeptide against a labeled competitor (e.g. agonist or antagonist). Further, these screening methods may test whether the candidate compound results in a signal generated by activation or inhibition of the polypeptide, using detection systems appropriate to the cells bearing the polypeptide. Inhibitors of activation are generally assayed in the presence of a known agonist and the effect on activation by the agonist by the presence of the candidate compound is observed. Further, the screening methods may simply comprise the steps of mixing a candidate compound with a solution containing a polypeptide of the present invention, to form a mixture, measuring an activity of the genes set forth in Table I in the mixture, and comparing activity of the mixture of the genes set forth in Table I to a control mixture which contains no candidate compound.

Polypeptides of the present invention may be employed in conventional low capacity screening methods and also in high-throughput screening (HTS) formats. Such HTS formats include not only the well-established use of 96- and, more recently, 384-well micotiter plates but also emerging methods such as the nanowell method described by Schullek et al, Anal Biochem., 246, 20-29, (1997). Fusion proteins, such as those made from Fc portion and polypeptide of the genes set forth in Table I, as hereinbefore described, can also be used for high-throughput screening assays to identify antagonists for the polypeptide of the present invention (see D. Bennett et al., J Mol Recognition, 8:52-58 (1995); and K. Johanson et al., J Biol Chem, 270(16):9459-9471 (1995)).

The polynucleotides, polypeptides and antibodies to the polypeptide of the present invention may also be used to configure screening methods for detecting the effect of added compounds on the production of mRNA and polypeptide in cells. For example, an ELISA assay may be constructed for measuring secreted or cell associated levels of polypeptide using monoclonal and polyclonal antibodies by standard methods known in the art. This can be used to discover agents that may inhibit or enhance the production of polypeptide (also called antagonist or agonist, respectively) from suitably manipulated cells or tissues.

A polypeptide of the present invention may be used to identify membrane bound or soluble receptors, if any, through standard receptor binding techniques known in the art. These include, but are not limited to, ligand binding and crosslinking assays in which the polypeptide is labeled with a radioactive isotope (for instance, ¹²⁵I), chemically modified (for instance, biotinylated), or fused to a peptide sequence suitable for detection or purification, and incubated with a source of the putative receptor (cells, cell membranes, cell supernatants, tissue extracts, bodily fluids). Other methods include biophysical techniques such as surface plasmon resonance and spectroscopy. These screening methods may also be used to identify agonists and antagonists of the polypeptide that compete with the binding of the polypeptide to its receptors, if any. Standard methods for conducting such assays are well understood in the art.

Examples of antagonists of polypeptides of the present invention include antibodies or, in some cases, oligonucleotides or proteins that are closely related to the ligands, substrates, receptors, enzymes, etc., as the case may be, of the polypeptide, e.g., a fragment of the ligands, substrates, receptors, enzymes, etc.; or a small molecule that bind to the polypeptide of the present invention but do not elicit a response, so that the activity of the polypeptide is prevented.

Screening methods may also involve the use of transgenic technology and the genes set forth in Table I. The art of constructing transgenic animals is well established. For example, the genes set forth in Table I may be introduced through microinjection into the male pronucleus of fertilized oocytes, retroviral transfer into pre- or post-implantation embryos, or injection of genetically modified, such as by electroporation, embryonic stem cells into host blastocysts. Particularly useful transgenic animals are so-called “knock-in” animals in which an animal gene is replaced by the human equivalent within the genome of that animal. Knock-in transgenic animals are useful in the drug discovery process, for target validation, where the compound is specific for the human target. Other useful transgenic animals are so-called “knock-out” animals in which the expression of the animal ortholog of a polypeptide of the present invention and encoded by an endogenous DNA sequence in a cell is partially or completely annulled. The gene knock-out may be targeted to specific cells or tissues, may occur only in certain cells or tissues as a consequence of the limitations of the technology, or may occur in all, or substantially all, cells in the animal. Transgenic animal technology also offers a whole animal expression-cloning system in which introduced genes are expressed to give large amounts of polypeptides of the present invention

Screening kits for use in the above described methods form a further aspect of the present invention. Such screening kits comprise:

(a) a polypeptide of the present invention;

(b) a recombinant cell expressing a polypeptide of the present invention;

(c) a cell membrane expressing a polypeptide of the present invention; or

(d) an antibody to a polypeptide of the present invention;

which polypeptide is preferably that set forth in the Sequence Listing.

It will be appreciated that in any such kit, (a), (b), (c) or (d) may comprise a substantial component.

### GLOSSARY

The following definitions are provided to facilitate understanding of certain terms used frequently hereinbefore.

“Antibodies” as used herein includes polyclonal and monoclonal antibodies, chimeric, single chain, and humanized antibodies, as well as Fab fragments, including the products of an Fab or other immunoglobulin expression library.

“Isolated” means altered “by the hand of man” from its natural state, i.e., if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated”, as the term is employed herein. Moreover, a polynucleotide or polypeptide that is introduced into an organism by transformation, genetic manipulation or by any other recombinant method is “isolated” even if it is still present in said organism, which organism may be living or non-living.

“Secreted protein activity or secreted polypeptide activity” or “biological activity of the secreted protein or secreted polypeptide” refers to the metabolic or physiologic function of said secreted protein including similar activities or improved activities or these activities with decreased undesirable side-effects. Also included are antigenic and immunogenic activities of said secreted protein.

“Secreted protein gene” refers to a polynucleotide comprising any of the attached nucleotide sequences or allelic variants thereof and/or their complements.

“Polynucleotide” generally refers to any polyribonucleotide (RNA) or polydeoxribonucleotide (DNA), which may be unmodified or modified RNA or DNA. “Polynucleotides” include, without limitation, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term “polynucleotide” also includes DNAs or RNAs containing one or more modified bases and DNAs or RNAs with backbones modified for stability or for other reasons. “Modified” bases include, for example, tritylated bases and unusual bases such as inosine. A variety of modifications may be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

“Polypeptide” refers to any polypeptide comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids. “Polypeptides” include amino acid sequences modified either by natural processes, such as post-translational processing, or by chemical modification techniques that are well known in the art. Such modifications are well described in basic texts and in more detailed monographs, as well as in a voluminous research literature. Modifications may occur anywhere in a polypeptide, including the peptide backbone, the amino acid side-chains and the amino or carboxyl termini. It will be appreciated that the same type of modification may be present to the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from post-translation natural processes or may be made by synthetic methods. Modifications include acetylation, acylation, ADP-ribosylation, amidation, biotinylation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination (see, for instance, Proteins—Structure and Molecular Properties, 2nd Ed., T. E. Creighton, W.H. Freeman and Company, New York, 1993; Wold, F., Post-translational Protein Modifications: Perspectives and Prospects, 1-12, in Post-translational Covalent Modification of Proteins, B. C. Johnson, Ed., Academic Press, New York, 1983; Seifter et al., “Analysis for protein modifications and nonprotein cofactors”, Meth Enzymol, 182, 626-646, 1990, and Rattan et al., “Protein Synthesis: Post-translational Modifications and Aging”, Ann NY Acad Sci, 663, 48-62, 1992).

“Fragment” of a polypeptide sequence refers to a polypeptide sequence that is shorter than the reference sequence but that retains essentially the same biological function or activity as the reference polypeptide. “Fragment” of a polynucleotide sequence refers to a polynucleotide sequence that is shorter than the reference sequence set forth in the Sequence Listing.

“Variant” refers to a polynucleotide or polypeptide that differs from a reference polynucleotide or polypeptide, but retains the essential properties thereof. A typical variant of a polynucleotide differs in nucleotide sequence from the reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from the reference polypeptide. Generally, alterations are limited so that the sequences of the reference polypeptide and the variant are closely similar overall and, in many regions, identical. A variant and reference polypeptide may differ in amino acid sequence by one or more substitutions, insertions, deletions in any combination. A substituted or inserted amino acid residue may or may not be one encoded by the genetic code. Typical conservative substitutions include Gly, Ala; Val, Ile, Leu; Asp, Glu; Asn, Gln; Ser, Thr; Lys, Arg; and Phe and Tyr. A variant of a polynucleotide or polypeptide may be naturally occurring such as an allele, or it may be a variant that is not known to occur naturally. Non-naturally occurring variants of polynucleotides and polypeptides may be made by mutagenesis techniques or by direct synthesis. Also included as variants are polypeptides having one or more post-translational modifications, for instance glycosylation, phosphorylation, methylation, ADP ribosylation and the like. Embodiments include methylation of the N-terminal amino acid, phosphorylations of serines and threonines and modification of C-terminal glycines.

“Allele” refers to one of two or more alternative forms of a gene occurring at a given locus in the genome.

“Polymorphism” refers to a variation in nucleotide sequence (and encoded polypeptide sequence, if relevant) at a given position in the genome within a population.

“Single Nucleotide Polymorphism” (SNP) refers to the occurrence of nucleotide variability at a single nucleotide position in the genome, within a population. An SNP may occur within a gene or within intergenic regions of the genome. SNPs can be assayed using Allele Specific Amplification (ASA). For the process at least 3 primers are required. A common primer is used in reverse complement to the polymorphism being assayed. This common primer can be between 50 and 1500 bps from the polymorphic base. The other two (or more) primers are identical to each other except that the final 3′ base wobbles to match one of the two (or more) alleles that make up the polymorphism. Two (or more) PCR reactions are then conducted on sample DNA, each using the common primer and one of the Allele Specific Primers.

“Splice Variant” as used herein refers to cDNA molecules produced from RNA molecules initially transcribed from the same genomic DNA sequence but which have undergone alternative RNA splicing. Alternative RNA splicing occurs when a primary RNA transcript undergoes splicing, generally for the removal of introns, which results in the production of more than one mRNA molecule each of that may encode different amino acid sequences. The term splice variant also refers to the proteins encoded by the above cDNA molecules.

“Identity” reflects a relationship between two or more polypeptide sequences or two or more polynucleotide sequences, determined by comparing the sequences. In general, identity refers to an exact nucleotide to nucleotide or amino acid to amino acid correspondence of the two polynucleotide or two polypeptide sequences, respectively, over the length of the sequences being compared.

“% Identity”—For sequences where there is not an exact correspondence, a “% identity” may be determined. In general, the two sequences to be compared are aligned to give a maximum correlation between the sequences. This may include inserting “gaps” in either one or both sequences, to enhance the degree of alignment. A % identity may be determined over the whole length of each of the sequences being compared (so-called global alignment), that is particularly suitable for sequences of the same or very similar length, or over shorter, defined lengths (so-called local alignment), that is more suitable for sequences of unequal length.

“Similarity” is a further, more sophisticated measure of the relationship between two polypeptide sequences. In general, “similarity” means a comparison between the amino acids of two polypeptide chains, on a residue by residue basis, taking into account not only exact correspondences between a between pairs of residues, one from each of the sequences being compared (as for identity) but also, where there is not an exact correspondence, whether, on an evolutionary basis, one residue is a likely substitute for the other. This likelihood has an associated “score” from which the “% similarity” of the two sequences can then be determined.

Methods for comparing the identity and similarity of two or more sequences are well known in the art. Thus for instance, programs available in the Wisconsin Sequence Analysis Package, version 9.1 (Devereux J et al, Nucleic Acids Res, 12, 387-395, 1984, available from Genetics Computer Group, Madison, Wis., USA), for example the programs BESTFIT and GAP, may be used to determine the % identity between two polynucleotides and the % identity and the % similarity between two polypeptide sequences. BESTFIT uses the “local homology” algorithm of Smith and Waterman (J Mol Biol, 147,195-197, 1981, Advances in Applied Mathematics, 2, 482489, 1981) and finds the best single region of similarity between two sequences. BESTFIT is more suited to comparing two polynucleotide or two polypeptide sequences that are dissimilar in length, the program assuming that the shorter sequence represents a portion of the longer. In comparison, GAP aligns two sequences, finding a “maximum similarity”, according to the algorithm of Neddleman and Wunsch (J Mol Biol, 48, 443453, 1970). GAP is more suited to comparing sequences that are approximately the same length and an alignment is expected over the entire length. Preferably, the parameters “Gap Weight” and “Length Weight” used in each program are 50 and 3, for polynucleotide sequences and 12 and 4 for polypeptide sequences, respectively. Preferably, % identities and similarities are determined when the two sequences being compared are optimally aligned.

Other programs for determining identity and/or similarity between sequences are also known in the art, for instance the BLAST family of programs (Altschul S F et al, J Mol Biol, 215, 403-410, 1990, Altschul S F et al, Nucleic Acids Res., 25:389-3402, 1997, available from the National Center for Biotechnology Information (NCBI), Bethesda, Md., USA and accessible through the home page of the NCBI at www.ncbi.nlm.nih.gov) and FASTA (Pearson W R, Methods in Enzymology, 183, 63-99, 1990; Pearson W R and Lipman D J, Proc Nat Acad Sci USA, 85,2444-2448,1988, available as part of the Wisconsin Sequence Analysis Package).

Preferably, the BLOSUM62 amino acid substitution matrix (Henikoff S and Henikoff J G, Proc. Nat. Acad Sci. USA, 89, 10915-10919, 1992) is used in polypeptide sequence comparisons including where nucleotide sequences are first translated into amino acid sequences before comparison.

Preferably, the program BESTFIT is used to determine the % identity of a query polynucleotide or a polypeptide sequence with respect to a reference polynucleotide or a polypeptide sequence, the query and the reference sequence being optimally aligned and the parameters of the program set at the default value, as hereinbefore described.

“Identity Index” is a measure of sequence relatedness which may be used to compare a candidate sequence (polynucleotide or polypeptide) and a reference sequence. Thus, for instance, a candidate polynucleotide sequence having, for example, an Identity Index of 0.95 compared to a reference polynucleotide sequence is identical to the reference sequence except that the candidate polynucleotide sequence may include on average up to five differences per each 100 nucleotides of the reference sequence. Such differences are selected from the group consisting of at least one nucleotide deletion, substitution, including transition and transversion, or insertion. These differences may occur at the 5′ or 3′ terminal positions of the reference polynucleotide sequence or anywhere between these terminal positions, interspersed either individually among the nucleotides in the reference sequence or in one or more contiguous groups within the reference sequence- In other words, to obtain a polynucleotide sequence having an Identity Index of 0.95 compared to a reference polynucleotide sequence, an average of up to 5 in every 100 of the nucleotides of the in the reference sequence may be deleted, substituted or inserted, or any combination thereof, as hereinbefore described. The same applies mutatis mutandis for other values of the Identity Index, for instance 0.96, 0.97, 0.98 and 0.99.

Similarly, for a polypeptide, a candidate polypeptide sequence having, for example, an Identity Index of 0.95 compared to a reference polypeptide sequence is identical to the reference sequence except that the polypeptide sequence may include an average of up to five differences per each 100 amino acids of the reference sequence. Such differences are selected from the group consisting of at least one amino acid deletion, substitution, including conservative and non-conservative substitution, or insertion. These differences may occur at the amino- or carboxy-terminal positions of the reference polypeptide sequence or anywhere between these terminal positions, interspersed either individually among the amino acids in the reference sequence or in one or more contiguous groups within the reference sequence. In other words, to obtain a polypeptide sequence having an Identity Index of 0.95 compared to a reference polypeptide sequence, an average of up to 5 in every 100 of the amino acids in the reference sequence may be deleted, substituted or inserted, or any combination thereof, as hereinbefore described. The same applies mutatis mnutatidis for other values of the Identity Index, for instance 0.96, 0.97, 0.98 and 0.99.

The relationship between the number of nucleotide or amino acid differences and the Identity Index may be expressed in the following equation:

nₐ≦xₐ−(xₐ·I),

in which:

nₐ is the number of nucleotide or amino acid differences,

xₐ is the total number of nucleotides or amino acids in a sequence set forth in the Sequence Listing,

I is the Identity Index,

· is the symbol for the multiplication operator, and in which any non-integer product of xₐ and I is rounded down to the nearest integer prior to subtracting it from xₐ.

“Homolog” is a generic term used in the art to indicate a polynucleotide or polypeptide sequence possessing a high degree of sequence relatedness to a reference sequence. Such relatedness may be quantified by determining the degree of identity and/or similarity between the two sequences as hereinbefore defined. Falling within this generic term are the terms “ortholog”, and “paralog”. “Ortholog” refers to a polynucleotide or polypeptide that is the functional equivalent of the polynucleotide or polypeptide in another species. “Paralog” refers to a polynucleotide or polypeptide that within the same species which is functionally similar.

“Fusion protein” refers to a protein encoded by two, often unrelated, fused genes or fragments thereof. In one example, EP-A-0 464 533-A discloses fusion proteins comprising various portions of constant region of immunoglobulin molecules together with another human protein or part thereof. In many cases, employing an immunoglobulin Fc region as a part of a fusion protein is advantageous for use in therapy and diagnosis resulting in, for example, improved pharmacokinetic properties [see, e.g., EP-A 0232 262]. On the other hand, for some uses it would be desirable to be able to delete the Fc part after the fusion protein has been expressed, detected and purified.

All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

TABLE I

| | | | Corresponding |
|---|---|---|---|
| | GSK | Nucleic Acid | Protein |
| Gene Name | Gene ID | SEQ ID NO's | SEQ ID NO's |
| sbg123493SLITa | 123493 | SEQ ID NO: 1 | SEQ ID NO: 34 |
| sbg14936EGFa | 14936 | SEQ ID NO: 2 | SEQ ID NO: 35 |
| | | SEQ ID NO: 3 | SEQ ID NO: 36 |
| SBh80018.cyastin- | 80018 | SEQ ID NO: 4 | SEQ ID NO: 37 |
| related | | | |
| SBh74552.trypsinogen | 74552 | SEQ ID NO: 5 | SEQ ID NO: 38 |
| | | SEQ ID NO: 6 | SEQ ID NO: 39 |
| sbg90060IGFBP | 90060 | SEQ ID NO: 7 | SEQ ID NO: 40 |
| | | SEQ ID NO: 8 | SEQ ID NO: 41 |
| sbg97078ANGIOa | 97078 | SEQ ID NO: 9 | SEQ ID NO: 42 |
| | | SEQ ID NO: 10 | SEQ ID NO: 43 |
| sbg68091CMP | 68091 | SEQ ID NO: 11 | SEQ ID NO: 44 |
| | | SEQ ID NO: 12 | SEQ ID NO: 45 |
| sbg18525LRR | 18525 | SEQ ID NO: 13 | SEQ ID NO: 46 |
| SBh45597.trypsin | 45597 | SEQ ID NO: 14 | SEQ ID NO: 47 |
| inhibitor | | SEQ ID NO: 15 | SEQ ID NO: 48 |
| sbg34640CALa | 34640 | SEQ ID NO: 16 | SEQ ID NO: 49 |
| | | SEQ ID NO: 17 | SEQ ID NO: 50 |
| sbg14849LO | 14849 | SEQ ID NO: 18 | SEQ ID NO: 51 |
| SBh35812.CALGIZZ | 35812 | SEQ ID NO: 19 | SEQ ID NO: 52 |
| ARIN | | SEQ ID NO: 20 | SEQ ID NO: 53 |
| sbg37967ECMPa | 37967 | SEQ ID NO: 21 | SEQ ID NO: 54 |
| | | SEQ ID NO: 22 | SEQ ID NO: 55 |
| sbg15037SER | 15037 | SEQ ID NO: 23 | SEQ ID NO: 56 |
| sbg23161EGFa | 23161 | SEQ ID NO: 24 | SEQ ID NO: 57 |
| | | SEQ ID NO: 25 | SEQ ID NO: 58 |
| sbg82008TGFa | 82008 | SEQ ID NO: 26 | SEQ ID NO: 59 |
| sbg82008TGFb | 82008 | SEQ ID NO: 27 | SEQ ID NO: 60 |
| sbg27142IGBb | 27142 | SEQ ID NO: 28 | SEQ ID NO: 61 |
| | | SEQ ID NO: 29 | SEQ ID NO: 62 |
| sbg239881TAGL | 239881 | SEQ ID NO: 30 | SEQ ID NO: 63 |
| | | SEQ ID NO: 31 | SEQ ID NO: 64 |
| sbg248602CHP | 248602 | SEQ ID NO: 32 | SEQ ID NO: 65 |
| sbg219473HNKS | 219473 | SEQ ID NO: 33 | SEQ ID NO: 66 |

TABLE II

| | | | | Cell |
|---|---|---|---|---|
| | | Closest Polynuclotide by | Closest Polypeptide by | Localization |
| Gene Name | Gene Family | homology | homology | (by homology) |
| sbg123493S | Slit-like | SC: AL157714 | Rat slit1 protein, gi: | Membrane- |
| LITa | protein | Submitted (20 Jan. 2001) | 4585574 | bound |
| | | by Sanger Centre, Hinxton, | Brose K, Bland KS, Wang | |
| | | Cambridgeshire, CB10 | KH, Arnott D, Henzel W, | |
| | | 1SA, UK. | Goodman CS, Tessier- | |
| | | | Lavigne M, Kidd T. Cell | |
| | | | 1999 Mar. 19; 96(6):795- | |
| | | | 806. | |
| sbg14936EG | EGF-Like 2 | GB: Z97832 | Mouse EGF-related | Secreted |
| Fa | family of | Submitted (01 Feb. 2000) | protein SCUBE1, gi: | |
| | polypeptides | by Sanger Centre, Hinxton, | 10998440 | |
| | | Cambridgeshire, CB10 | Submitted (08 Jun. 2000) | |
| | | 1SA, UK. | by Mammalian Genetics | |
| | | | Unit, MRC Harwell, | |
| | | | Chilton, Didcot, Oxon | |
| | | | OX11 0RD, United | |
| | | | Kingdom. | |
| SBh80018.c | Cystatin- | GB: AL121894 | Mouse cystatin T (Zcys3), | Secreted |
| yastin- | related | Submitted (25 Oct. 2000) | geneseqp: Y96576 | |
| related | epididymal | by Sanger Centre, Hinxton, | Patented by | |
| | spermatogenic | Cambridgeshire, CB10 | ZYMOGENETICS INC | |
| | protein | 1SA, UK. | Patent number and and | |
| | | | publication date: | |
| | | | WO200031264-A2, 02 | |
| | | | Jun. 2000 | |
| SBh74552- | Trypsinogen | GB: U66059 | Mouse Trypsinogen, | Secreted |
| .trypsinogen | | Rowen, L., Koop, B. F. and | gi2358070 | |
| | | Hood, L. | Rowen, L., Smit, A. F. A. | |
| | | Science 272 (5269), 1755- | and Hood, L., Submitted | |
| | | 1762 (1996). | (20 Jul. 1997) | |
| | | | Department of Molecular | |
| | | | Biotechnology, Box | |
| | | | 357730 University of | |
| | | | Washington, Seattle, | |
| | | | Washington 98195, USA | |
| sbg90060- | Insulin-like | GB: AC020916 | Protein PRO332, | Secreted |
| IGFBP | growth factor | Direct submitted (12 Jan. | geneseqp: Y13396 | |
| | binding | 2000) by Production | Patented by Genetech Inc | |
| | protein | Sequencing Facility, DOE | Patent Number and | |
| | (IGFBP) | Joint Genome Institute, | publication date: | |
| | | 2800 Mitchell Drive, | WO9914328-A2, 25 Mar. | |
| | | Walnut Creek, CA 94598, | 1999 | |
| | | USA | | |
| sbg97078- | Angiotensin | GB: AC011476 | Human hypothetical | Membrane- |
| ANGIOa | II/vasopressin | Direct submitted (07 Oct. | protein FLJ20510: | bound |
| | receptor | 1999) by Production | gi: 8923473. Submitted | |
| | | Sequencing Facility, DOE | (02 Nov. 2000) by Sumio | |
| | | Joint Genome Institute, | Sugano, Institute of | |
| | | 2800 Mitchell Drive, | Medical Science, | |
| | | Walnut Creek, CA 94598, | University of Tokyo, | |
| | | USA. | Department of Virology; | |
| | | | Shirokane-dai, 4-6-1, | |
| | | | Minato-ku, Tokyo 1O8- | |
| | | | 8639 | |
| sbg68091- | Cartilage | GB: AC006356 | Human zkun5 protein, | Secreted |
| CMP | matrix | Direct Submitted (29 | geneseqp: Y52597. | |
| | protein | May 1999) by Genome | Patented by | |
| | | Sequencing Center, | ZYMOGENETICS INC. | |
| | | Washington University | Patent number and and | |
| | | School of Medicine, 4444 | publication date: | |
| | | Forest Park Parkway, St. | WO9961615-A1, 02 Dec. | |
| | | Louis, MO 63108, USA | 1999 | |
| sbg18525- | Leucine-rich | GB: AC016030 | Human KIAA0416 | Membrane- |
| LRR | repeat (LLR) | Direct submitted (19 | protein, gi: 7662102. | bound |
| | | Nov. 1999) by Whitehead | Ishikawa, K., Nagase. T., | |
| | | Institute/MIT Center for | Nakajima, D., Seki, N., | |
| | | Genome Research, 320 | Ohira, M., Miyajima, N., | |
| | | Charles Street, Cambridge, | Tanaka, A., Kotani, H., | |
| | | MA 02141, USA | Nomura, N. and Ohara, O. | |
| | | | 1997. DNA Res. 4:307- | |
| | | | 313. | |
| SBh45597- | Rab | SC: Z84479 | Human RAS like | Cytosolic |
| .trypsin | subfamily of | Submitted (16 Oct. 1997) | GTPASE, gi: 3036779. | |
| inhibitor | Ras-like | by Sanger Centre, | Submitted (16 Oct. | |
| | GTPase | Wellcome Trust Genome | 1997) Sanger Centre, | |
| | | Campus, Hinxton, | Wellcome Trust Genome | |
| | | Cambridgeshire, CB10 | Campus, Hinxton, | |
| | | 1SA, UK. | Cambridgeshire, CB10 | |
| | | | 1SA, UK. | |
| sbg34640- | Calgizzarin | GB: AC006483 | Human calgizzarin, | Cytosolic |
| CALa | (endothelial | Sulston, J. E. and | gi: 1710818. Tanaka, M., | |
| | monocyte- | Waterston, R | Adzuma, K., Iwami, M., | |
| | activating | Genome Res. 8 (11), 1097- | Yoshimoto. K., | |
| | polypeptide) | 1108 (1998) | Monden. Y. and | |
| | | | Itakura, M. Cancer Lett. | |
| | | | 89 (2), 195-200 (1995). | |
| sbg14849LO | Lysyl | GB: AC005033 | Mouse lysyl oxidase- | Secreted |
| | oxidase-like | Direct Submitted (12 Jun. | related protein 2, | |
| | | 1998) by Genome | gi: 7305239. Jang. W., | |
| | | Sequencing Center, | Hua, A., Spilson. S. V., | |
| | | Washington University | Miller, W., Roe, B. A. and | |
| | | School of Medicine, 4444 | Meisler, M. H., 1999, | |
| | | Forest Park Parkway, St. | Genome Res. 9:53-61. | |
| | | Louis, MO 63108, USA. | | |
| SBh35812- | Calgizzarin | GB: AL133399 | Mouse calgizzarin, | Cytosolic |
| .CALGIZ- | (endothelial | Submitted (08 Feb. 2000) | gi: 1710819. Submitted | |
| ZARIN | monocyte- | by Sanger Centre, Hinxton, | (27 Nov. 1995) Keith A. | |
| | activating | Cambridgeshire, CB10 | Houck, Biomolecular | |
| | polypeptide) | 1SA, UK. | Research, Sphinx | |
| | | | Pharmaceuticals Corp., | |
| | | | 4615 University Dr., | |
| | | | Durham, NC 27707, USA | |
| sbg37967- | Extracellular | JENA: X57A-X51X57A- | Human extracellular | Secreted |
| ECMPa | matrix | X51 found at Jena Genome | matrix protein 2, | |
| | protein 2 | Sequencing Center | gi: 4557543. Nishiu. J., | |
| | | | Tanaka, T. and | |
| | | | Nakamura, Y. Genomics | |
| | | | 52, 378-381 (1998) | |
| sbg15037- | Serine | GB: AC005570 | A long isoform of human | Secreted |
| SER | protease | Direct submitted (01 Sep. | HELA2 protein, W77297 | |
| | | 1998) Center for Human | Patented by Amrad | |
| | | Genome Studies, DOE | Operations Pty Ltd. Patent | |
| | | Joint Genome Institute, | number and and | |
| | | Los Alamos National | publication date: | |
| | | Laboratory, MS M888, Los | WO9836054-A1, 20- | |
| | | Alamos, NM 87545, USA. | Aug. 1998 | |
| sbg23161- | Extracellular/ | GB: Z99756, GB: Z82214 | Mouse EGF-related | Secreted |
| EGFa | epidermal | Submitted (08 Dec. 1999) | protein SCUBE1 | |
| | growth factor | by Sanger Centre, Hinxton, | gi: 10998440. | |
| | | Cambridgeshire, CB10 | Grimmond, S., Larder, R., | |
| | | 1SA, UK. | Van Hateren, N., | |
| | | | Siggers, P., | |
| | | | Hulsebos, T. J . M., | |
| | | | Arkell, R. and Greenfield, | |
| | | | A. Genomics 70 (1), 74- | |
| | | | 81 (2000) | |
| sbg82008- | TGF beta | GB: AC008940.frag1. | A novel isolated and | Secreted |
| TGFa,b | (transforming | Submitted (03 Aug. 1999) | purified growth factor | |
| | growth factor | by Production Sequencing | (GF), Y16714. Patented | |
| | beta) | Facility, DOE Joint | by UNIV | |
| | | Genome Institute, 2800 | WASHINGTON. Patent | |
| | | Mitchell Drive, Walnut | number and and | |
| | | Creek, CA 94598, USA | publication date: | |
| | | | WO9914235, 25 Mar. 1999 | |
| sbg27142- | Immunoglobulin | GB: AC011846: | Mouse cell adhesion | Secreted |
| IGBb | superfamily | Submitted (15 Oct. 1999) | molecule, gi: 1862939. | |
| | | Whitehead Institute/MIT | Submitted (11 Dec. | |
| | | Center for Genome | 2000) Junya Toguchida, | |
| | | Research, 320 Charles | Kyoto University, | |
| | | Street, Cambridge, MA | Institute for Frontier | |
| | | 02141, USA | Medical Sciences; 53 | |
| | | GB: AC068507: | Kawahara-cho, Shogoin, | |
| | | Submitted (03 May 2000) | Sakyo-ku, Kyoto, Kyoto | |
| | | Whitehead Institute/MIT | 606-8507, Japan | |
| | | Center for Genome | | |
| | | Research, 320 Charles | | |
| | | Street, Cambridge, MA | | |
| | | 02141, USA | | |
| sbg239881- | Tag7-like | GB: AC011492 | Mouse TAGL-alpha | Secreted |
| TAGL | family | Direct submitted (07 Oct. | protein, gi: 10946624. | |
| | protein | 1999) by Production | Submitted (11 May | |
| | | Sequencing Facility, DOE | 1999) Laboratory of | |
| | | Joint Genome Institute, | Cancer Molecular | |
| | | 2800 Mitchell Drive, | Genetics, Institute of | |
| | | Walnut Creek, CA 94598, | Gene Biology, Russian | |
| | | USA. | Academy of Sciences, | |
| | | | 34/5 Vavilov Street, | |
| | | | Moscow 117334, Russia | |
| sbg248602- | Zinc | GB: AL035460 | Mouse metallocarboxy- | Secreted |
| CHP | Carboxy- | Direct submitted (20 Mar. | peptidase CPX-1, | |
| | peptidase | 2000) by Sanger Centre, | AAD15985. Lei. Y., | |
| | | Hinxton, Cambridgeshire, | Xin, X., Morgan, D., | |
| | | CB10 1SA, UK | Pintar, J. E. and | |
| | | | Fricker, L. D, 1999, DNA | |
| | | | Cell Biol. 18:175-185. | |
| sbg219473- | HNK- | GB: AP001087 | Human GalNAc 4-sulfo- | Membrane- |
| HNKS | sulfotrans- | Direct submitted (25 Jan. | transferase, gi: 11990885. | bound |
| | ferase | 2000) by the Institute of | Habuchi. O. and Okuda, T. | |
| | | Physical and Chemical | J. Biol. Chem. 275 (51), | |
| | | Research (RIKEN), | 40605-40613 (2000) | |
| | | Genomic Sciences Center | | |
| | | (GSC); Kitasato Univ., 1- | | |
| | | 15-1 Kitasato, Sagamihara, | | |
| | | Kanagawa 228-8555, | | |
| | | Japan. | | |

TABLE III

| Gene Name | Uses | Associated Diseases |
|---|---|---|
| sbg123493 | An embodiment of the invention may be the use of sbg123493- | Diseases in spinal cord, |
| -SLITa | SLITa, a secreted protein, to bind Robo receptors and have an | thyroid gland, ovary, |
| | evolutionarily conserved role in repulsive axon guidance and may be | prostate, renal gland, |
| | useful for the prevention and treatment of diseases in spinal cord, | small intestine, heart, |
| | thyroid gland, ovary, prostate, renal gland, small intestine, heart, | trachea, thymus, lymph |
| | trachea, thymus, lymph node, muscular system and colon. | node, muscular system |
| | sbg123493-SLITa may also be used in the treatment of pineal tumors | and colon, pineal tumors |
| | and alleviation of precocious puberty. | and alleviation of |
| | Close homologs of sbg123493-SLITa are rat protein-Slit protein and | precocious puberty |
| | pineal gland specific gene-1 protein. | |
| sbg14936- | An embodiment of the invention is the use of sbg14936-EGFa, a | Neurodegenerarive |
| EGFa | secreted protein, to treat colorectal carcinomas, and peptic ulcer | disorders, trauma, natural |
| | healing. The closest homologue to sbg14936-EGFa is high- | blinding, colorectal |
| | molecular-weight proteins with multiple EGF-like motifs. | carcinomas and peptic |
| | Polypeptides with EGF-like and/or cadherin-like repeats have been | ulcer healing |
| | used to stimulate the growth of various epidermal and epithelial | |
| | tissues in vivo and in vitro and of some fibroblasts in cell culture. | |
| SBh80018 | An embodiment of the invention is the use of SBh80018-cyastin- | Autoimmune disorder, |
| -.cyastin- | related to treat or prevent tissue damage associated with brain | hematopoietic disorder, |
| related | hemorrhage. | wound healing disorder, |
| | | viral and bacterial |
| | | infection, cancer, |
| | | neurological disorder, |
| | | brain haemorrhage, tissue |
| | | damage, inflammation, |
| | | and protection and |
| | | remodeling of the eye |
| SBh74552- | An embodiment of the invention is the use of SBh74552-trypsinogen | Autoimmune disorder, |
| trypsinogen | to treat clot formation induced by myocardial infarction and | hematopoietic disorder, |
| | reocclusion following angioplasty or pulmonary thromboembolism. | wound healing disorder, |
| | Close homologues to of SBh74552-trypsinogen are used to treat clot | viral and bacterial |
| | formation and for treating associated gastrointestinal and | infection, cancer, clot |
| | haematopoietic disorders. | formation in myocardial |
| | | infarction, reocclusion |
| | | following angioplasty or |
| | | pulmonary |
| | | thromboembolism, |
| | | gastrointestinal disorders |
| sbg90060- | An embodiment of the invention is the use of sbg90060-IGFBP, | Cancer, infection, |
| IGFBP | in the treatment of a wide range of disease states including | autoimmune disorder, |
| | cancer, diabetes, vascular disease, asthma, and growth disorders. | hematopoietic disorder, |
| | Close homologs of sbg90060-IGFBP are Insulin-like growth | wound healing |
| | factor (IGF) binding proteins (IGFBP). IGFBP when occupied | disorder, inflammation, |
| | by IGF, combines with an acid-labile glycoprotein subunit (ALS) | diabetes, vascular |
| | to form a high molecular weight complex. The IGFBPs regulate | disease, asthma, and |
| | somatic growth and cellular proliferation both in vivo and in | growth isorders |
| | vitro. The IGFBPs also appear to have emerging roles in the | |
| | mechanisms underlying human cancer. Future research on its | |
| | physiology may have advancements in the treatment of a wide | |
| | range of disease states including cancer, diabetes, vascular | |
| | disease, asthma, and growth disorders (Wetterau L A, Moore M G, | |
| | Lee K W, Shim M L, Cohen P, 1999, Mol Genet Metab 68:161- | |
| | 81). | |
| sbg97078- | An embodiment of the invention is the use of sbg97078- | Cancer, infection, |
| ANGIOa | ANGIOa, in treating hypertension, heart disease, and kidney | autoimmune disorder, |
| | disease, related to unbalanced levels of angiotensin II/vasopressin | hematopoietic disorder, |
| | receptors. | wound healing |
| | A close homolog of sbg97078-ANGIOa is angiotensin | disorder, inflammation |
| | H/vasopressin receptors. Angiotensin II/vasopressin receptors | hypertension, heart |
| | couple to adenylate cyclase and responds with equal sensitivity to | disease, and kidney |
| | Ang II and AVP. Ang II receptors respond to the | disease |
| | neurotransmitter angiotensin II whilst AVP receptors respond to | |
| | arginine vasopressin. Vasopressin receptor mediates many | |
| | central and peripheral actions of vasopressin, including | |
| | intracellular calcium mobilization. Thus the proteins, antibodies, | |
| | agonists and antagonists can be used for treating, e.g. | |
| | hypertension, heart disease, and kidney disease, related to | |
| | unbalanced levels of angiotensin II/vasopressin receptor (Howl J, | |
| | Wheatley M, 1995 Gen Pharmacol 26:1143-52; Grazzini E, | |
| | Boccara G, Joubert D, Trueba M, Durroux T, Guillon G, Gallo- | |
| | Payet N, Chouinard L, Payet M D, Serradeil Le Gal C, 1998 Adv | |
| | Exp Med Biol 449:325-34). | |
| sbg68091- | An embodiment of the invention is the use of sbg68091-CMP, in | Cancer, infection, |
| CMP | repairing damaged cartilage in joints, such as in osteoarthritis and | autoimmune disorder, |
| | rheumatoid arthritis. | hematopoietic disorder, |
| | A close homolog of sbg68091-CMP is Matrilin-1. The matrilin | wound healing |
| | family shares a common structure made up of von Willebrand | disorder, inflammation |
| | factor A domains, epidermal growth factor-like domains and a | rheumatoid arthritis, |
| | coiled coil alpha-helical module (Deak F, Wagener R, Kiss I, | and osteoarthritis. |
| | Paulsson M, 1999. Matrix Biol 18:55-64). Matrilin-1, cartilage | |
| | matrix protein (CMP), is a major component of the extracellular | |
| | matrix of nonarticular cartilage, and it binds to collagen. | |
| sbg18525- | An embodiment of the invention is the use of sbg18525-LRR a | Cancer, infection, |
| LRR | member of the leucine-rich repeat protein family, in | autoimmune disorder, |
| | immunization , protein-protein interactions, such as cell | hematopoietic disorder, |
| | adhesion or receptor-ligand binding and neuronal LRR may be | wound healing disorder, |
| | an important component of the pathophysiological response to | inflammation, |
| | brain injury. Close homologs of sbg18525-LRR are leucine- | gastrointestinal |
| | rich repeat (LRR) proteins such as connectin, slit, chaoptin, | ulceration, and diseases |
| | and toll. These proteins have important roles in neuronal | in spinal cord, thyroid |
| | development and the adult nervous system as cell adhesion | gland, heart, trachea, |
| | molecules (Taguchi A, Wanaka A, Mori T, Matsumoto K, Imai | thymus, lymph node, |
| | Y, Tagaki T, Tohyama M, 1996, Brain Res Mol Brain | muscular system, and |
| | Res; 35:31-4). At least one LRR was shown to be specifically | nervous system |
| | expressed on B cells, suggesting its role in immunization | |
| | (Miyake K, Yamashita Y, Ogata M, Sudo T, Kimoto M, 1995. | |
| | J Immunol 154:3333-40). Some studies have shown that brain | |
| | injury can cause over expression of neuronal LRR, suggesting | |
| | that neuronal LRR may be an important component of the | |
| | pathophysiological response to brain injury (Ishii N, Wanaka | |
| | A, Tohyama M, 1996, Brain Res Mol Brain Res 40:148-52).. | |
| SBh45597- | An embodiment of the invention is the use of SBh45597- | Acute respiratory |
| trypsin | trypsin inhibitor in vesicle targeting. The Rabs are a subfamily | disease, AIDs, allergy, |
| inhibitor | within the large group of small GTP-binding proteins and have | atherosclerosis, cancer, |
| | been showed to play a role in vesicle targeting. Like RAS, | biabetes, cerebral |
| | they cycle between active GTP-bound and inactive GDP-bound | neoplasm, immune |
| | forms with both transitions to require additional factors: | disorder, |
| | GTPase-activating proteins (GAPs) and guanine nucleotide | imflasmmatory |
| | exchange factors (GEFs). The GDP-bound form is also a target | disorder, rheumatoid |
| | for a GDI (GDP dissociation inhibitor), a slightly-misnamed | arthritis, viral infection. |
| | but remarkable protein which extracts the GDP-Rab (including | |
| | its very hydrophobic isoprenoid groups) from the membrane, | |
| | allowing it to return via the cytosol to its membrane of origin. | |
| | (Armstrong J. Int J Biochem Cell Biol 2000 Mar; 32(3):303-7). | |
| sbg34640- | An embodiment of the invention is the use of sbg34640-CALa, | Infections, cancers, |
| CALa | a secreted protein, in the diagnosis and treatment of cancer. | autoimmune disorders, |
| | Close homologues to sbg34640-CALa are S100 calcium- | wound healing disorder |
| | binding protein A11 (calgizzarin) and other EF-hand calcium | and hematopoietic |
| | binding proteins and more specifically to s-100/CABP like | disorder |
| | proteins. S100 calcium-binding protein A11 (calgizzarin) | |
| | binds two calcium ions per molecule with an affinity similar to | |
| | that of the s-100 proteins. s-100/CABP like proteins are useful | |
| | in diagnosis and treatment of cancer. (Fan, Y., Leung, D., | |
| | Houck, K. A., Yan, S., Kao, J. Calgizzarin (endothelial | |
| | monocyte-activating polypeptide ((EMAP) Submitted January | |
| | 1996 to the EMBL/GenBank/DDBJ databases. ACCESSION | |
| | NO: P50543.). | |
| sbg14849LO | An embodiment of the invention is the use of sbg14849LO in | Cancer, infection, |
| | the biogenesis of connective tissue matrices by crosslinking the | autoimmune disorder, |
| | extracellular matrix proteins, collagen and elastin or in the | hematopoietic disorder, |
| | treatment of osteoporotic bone. A close homologue of | wound healing disorder, |
| | sbg14849LO is lysyl oxidase (LO). LO is a cuproenzyme that | inflammation, fibrotic |
| | plays a critical role in the biogenesis of connective tissue | diseases, and metabolic |
| | matrices by crosslinking the extracellular matrix proteins, | bone diseases |
| | collagen and elastin. Levels of LO increase in many fibrotic | |
| | diseases, while expression of the enzyme is decreased in some | |
| | diseases related to impaired copper metabolism. Transforming | |
| | growth factor-beta, platelet-derived growth factor, angiotensin | |
| | II, retinoic acid, fibroblast growth factor, and altered serum | |
| | conditions can affect LO expression. It has also become | |
| | increasingly evident that LO may have other important | |
| | biological functions (Smith-Mungo LI, and Kagan HM, 1998, | |
| | Matrix Biol 16:387-98). In mineralizing tissues, a relatively | |
| | low level of lysyl hydroxylation results in low levels of | |
| | hydroxylysyl pyridinoline, and the occurrence of the largely | |
| | bone specific lysyl pyridinoline and pyrrolic cross-links (Knott | |
| | L, and Bailey A J, 1998, Bone 22:181-7). | |
| SBh35812- | An embodiment of the invention is the use of SBh35812- | Autoimmune disorder, |
| CALGIZ- | CALGIZ-ZARIN to activate host response mechanisms. Close | hematopoietic disorder, |
| ZARIN | homologues of SBh35812-CALGIZ-ZARIN are cytokines and | wound healing disorder, |
| | S-100 PROTEINS. | viral and bacterial |
| | | infection, cancer, |
| | | melanoma cance, |
| | | cerebral dysfunction |
| sbg37967- | An embodiment of the invention is the use of sbg37967- | Cancer, autoimmune |
| ECMPa | ECMPa, a secreted protein, in wound healing and treatment of | disease, inflammatory |
| | inflammatory diseases. A close homologue to sbg37967- | diseases, wound healing |
| | ECMPa is extracellular matrix protein 2 (pECM2). pECM2 | and hematopoietic |
| | expressed predominantly in adipose and female-specific tissues | disorder |
| | and its chromosomal localization to 9q22.3 and participates in | |
| | protein-protein interactions and/or cell-ECM recognition | |
| | processes (Nishiu, J., Tanaka, T. and Nakamura, Y. 1998. | |
| | Genomics 52, 378-381). | |
| sbg15037- | An embodiment of the invention is the use of sbg15037-SER in | Cancer, including |
| SER | the diagnosis of testicular tumors. sbg15037-SER is a | testicular turmors, |
| | membrane-type serine protease which shows a trypsin-like | infection, autoimmune |
| | cleavage activity. A close homologue to sbg15037-SER is | disorder, hematopoietic |
| | testisin, a new human serine proteinase, which is abundantly | disorder, wound healing |
| | expressed only in the testis and is lost in testicular tumors. | disorders, and |
| | These findings about testisin demonstrate a new cell surface | inflammation |
| | serine proteinase, loss of which may have a role in the | |
| | progression of testicular tumors of germ cell origin. (Hooper | |
| | J D, Nicol D L, Dickinson J L, Eyre H J, Scarman A L, Normyle | |
| | J F, Stuttgen M A, Douglas M L, Loveland K A, Sutherland G R, | |
| | and Antalis T M, 1999, Cancer Res 59:3199-205). | |
| sbg23161- | An embodiment of the invention is the use of sbg23161-EGFa, | Cancer, autoimmune |
| EGFa | a secreted protein, in regulating vascular smooth muscle cell | disorders, wound healing |
| | proliferation, e.g. for enhancing neurological functions or | disorders, infections, and |
| | treating neoplasia and other disorders. A close homologue to | hemotopoietic disorders |
| | sbg23161-EGFa is human extracellular/epidermal growth | |
| | factor-like protein (EEGF). This EEGF protein is useful for | |
| | regulating vascular smooth muscle cell proliferation, e.g. for | |
| | enhancing neurological functions or treating neoplasia and | |
| | other disorders (LI HS and OLSEN H, New isolated | |
| | extracellular/epidermal growth factor, Accession Number | |
| | W79739, HUMAN GENOME SCI INC). | |
| sbg82008- | An embodiment of the invention is the use of sbg82008- | Cancer (eg., lymphoma, |
| TGFa,b | TGFa,b in growth control and hence the etiology of cancer, cell | leukemia, renal cell |
| | differentiation and development. sbg82008-TGFa,b contains | carcinoma, melanoma, |
| | the Prosite consensus pattern (PDOC00223) for TGF beta | lung cancer), infection |
| | family members. | (viral disease, (eg hepatitis |
| | Close homologues of sbg82008-TGFa,b are TGF-beta proteins. | A and C), parasitic disease, |
| | TGF-beta proteins are known to be involved in growth control | bacterial disease), |
| | and hence the etiology of cancer (Anticancer Res 1999 Nov- | inflammation, autoimmune |
| | Dec; 19(6A):4791-807), cell differentiation and development. A | disorder (eg multiple |
| | TGF-beta signaling pathway constitutes a tumor suppressor | sclerosis, Type I diabetes), |
| | path (Cytokine Growth Factor Rev 2000 Apr. 1; 11(1-2):159- | infertility, miscarriage, |
| | 168). | hematopoietic disorder, |
| | | wound healing disorder, |
| | | inflammatory diseases, |
| | | inflammatory bowel |
| | | disease, cystic fibrosis, |
| | | immune deficiency, |
| | | thrombocytopenia, chronic |
| | | obstructive pulmonary |
| | | disease |
| sbg27142- | An embodiment of the invention is the use of sbg27142-IGBb | Cancer, infection diseases, |
| IGBb | in the diagnosis and/or treatment of cancer and autoimmune | autoimmune disorder, |
| | disorders of the nervous system. A close homologue to | wound healing disorder |
| | sbg27142-IGBb is the mouse cell adhesion molecule | and hematopoietic disorder |
| | (gi: 11862939) that has been associated with transformation of | |
| | osteoblasts and the mouse gene Punc that is expressed | |
| | predominantly in the developing nervous system (Salbaum, | |
| | J. M. 1998 Mech. Dev. 71 (1-2), 201-204). | |
| sbg239881- | An embodiment of the invention is the use of sbg239881- | Cancer, infection, |
| TAGL | TAGL to inhibit tumor growth and induce apoptosis and/or | autoimmune disorder, |
| | may also be useful as probes for gene mapping and detection of | hematopoietic disorder, |
| | tag7 gene expression. Close homologues to sbg239881-TAGL | wound healing disorders |
| | and its promoter region are genes of the tumor necrosis factor | |
| | (TNF). The tag7 coding sequences are also useful as probes | |
| | for gene mapping and detection of tag7 gene expression | |
| | (Kiselev S L, Kustikova O S, Korobko E V, Prokhortchouk E B, | |
| | Kabishev A A, Lukanidin E M, Georgiev G P, 1998, J Biol | |
| | Chem 273:18633-9). | |
| sbg248602- | Due to the carboxypeptidase activity required for processing of | Cancer, infection, |
| CHP | various neuropeptides and hormones, an embodiment of the | autoimmune disorder, |
| | invention is the use of sbg248602-CHP in treatments of | hematopoietic disorder, |
| | neurodegenerative disorders and developmental abnormalities. | wound healing disorders, |
| | Close homologues to sbg248602-CHP are peptidases that | inflammation, |
| | catalyze the removal of c-terminal basic amino acid residues, | neurodegenerative |
| | and is involved in processing of neuropeptides and hormones | disorders, and |
| | in secretory vesicles (Manser E, Fernandez D, Loo L, Goh P Y, | developmental |
| | Monfries C, Hall C, and Lim L, 1990, Biochem J 267:517-25). | abnormalities |
| | Some enzymes from this family have been isolated in multiple | |
| | forms from both soluble and membrane-bound compartments, | |
| | and are demonstrated to co-secrete with peptides from | |
| | pancreatic and adrenal cells. Single mRNA species have been | |
| | shown to yield multiple forms of similar peptidases (Manser E, | |
| | Fernandez D, and Lim L, 1991, Biochem J 280:695-701). | |
| sbg219473- | An embodiment of the invention may be the use of sbg219473- | Cancer, infection, |
| HNKS | HNKS in the development of the nervous system, and may also | autoimmune disorder, |
| | be involved in the preferential reinervation of muscle nerves by | hematopoietic disorder, |
| | motor axons after lesion. Close homologues to sbg219473- | wound healing disorders, |
| | HNKS are sulfotransferases. Sulforransferase is considered to | inflammation, and |
| | be the key enzyme in the biosynthesis of the HNK-1 | peripheral neuropathies |
| | carbohydrate epitope, which is expressed on several neural | |
| | adhesion glycoproteins and as a glycolipid, and is involved in | |
| | cell interactions (Bakker, H., Friedmann, I., Oka, S., | |
| | Kawasaki. T., Nifant'ev, N., Schachner, M., and Mantei, N., | |
| | 1997, J. Biol. Chem. 272:29942-29946). The HNK-1 epitope is | |
| | spatially and temporally regulated during the development of | |
| | the nervous system. The biological function of the HNK-1 | |
| | sulfotransferase may be related to the development of the | |
| | nervous system, and also may be involved in the preferential | |
| | reinervation of muscle nerves by motor axons after lesion | |
| | (Jungalwala F B, 1994, Neurochem Res 19:945-57). | |

TABLE IV
Quantitative, Tissue-specific mRNA expression detected using SybrMan

| | Tissue-Specific mRNA Expression | | | | |
|---|---|---|---|---|---|
| | (copies per ng mRNA; avg. ± range for 2 data points per tissue) | | | | |
| | | | | | Kid- |
| Gene Name | Brain | Heart | Lung | Liver | ney |
| sbg123493- | 9 ± 3 | 70 ± 31 | 13 ± 3 | −1 ± 1 | 41 ± 16 |
| SLITa | | | | | |
| sbg14936- | 516 ± 34 | 2424 ± 72 | 550 ± 56 | 129 ± 7 | 1825 ± 6 |
| EGFa | | | | | |
| SBh80018- | 1 ± 0 | 2 ± 1 | 0 ± 0 | −7 ± 4 | 2 ± 3 |
| .cyastin- | | | | | |
| related | | | | | |
| SBh74552- | −1 ± 1 | 7 ± 1 | 9 ± 1 | −10 ± 1 | 1 ± 3 |
| .trypsinogen | | | | | |
| sbg90060- | 366 ± 17 | 659 ± 36 | 784 ± 64 | 53 ± 7 | 1035 ± 189 |
| IGFBP | | | | | |
| sbg97078- | 15 ± 1 | 16 ± 7 | 58 ± 3 | −6 ± 1 | 18 ± 1 |
| ANGIOa | | | | | |
| sbg68091- | 1360 ± 30 | 3596 ± 59 | 1846 ± 271 | 248 ± 18 | 2596 ± 146 |
| CMP | | | | | |
| sbg18525- | 4290 ± 157 | 367 ± 6 | 47 ± 4 | 7 ± 0 | 263 ± 10 |
| LRR | | | | | |
| SBh45597- | 59 ± 12 | 58 ± 7 | 44 ± 1 | 22 ± 1 | 106 ± 21 |
| .trypsin | | | | | |
| inhibitor | | | | | |
| sbg34640- | 3006 ± 11 | 30001 ± 197 | 98054 ± 1290 | 4166 ± 228 | 39196 ± 1674 |
| CALa | | | | | |
| sbg14849- | 508 ± 23 | 862 ± 13 | 631 ± 8 | 51 ± 5 | 251 ± 24 |
| LO | | | | | |
| SBh35812.- | 345 ± 1 | 20 ± 1 | 11 ± 1 | −3 ± 7 | 45 ± 1 |
| CALGIZ- | | | | | |
| ZARIN | | | | | |
| sbg37967- | 72 ± 5 | 26 ± 10 | 24 ± 8 | 3 ± 9 | 45 ± 0 |
| ECMPa | | | | | |
| sbg15037- | 291 ± 9 | 256 ± 24 | 284 ± 18 | 302 ± 7 | 312 ± 6 |
| SER | | | | | |
| sbg23161- | 150 ± 1 | 142 ± 9 | 2063 ± 68 | 348 ± 20 | 1184 ± 80 |
| EGFa | | | | | |
| sbg82008- | 1542 ± 96 | 651 ± 49 | 858 ± 37 | 555 ± 50 | 818 ± 248 |
| TGFa,b | | | | | |
| sbg2714- | 526 ± 37 | 505 ± 8 | 115 ± 5 | −6 ± 9 | 91 ± 3 |
| 2IGBb | | | | | |
| sbg23988- | 3 ± 1 | 2 ± 0 | 6 ± 1 | 2816 ± 28 | 6 ± 1 |
| 1TAGL | | | | | |
| sbg248602- | 134 ± 10 | 989 ± 16 | 539 ± 3 | 3 ± 5 | 1335 ± 16 |
| CHP | | | | | |
| sbg219473- | 175 ± 32 | 1075 ± 81 | 2522 ± 91 | 473 ± 35 | 453 ± 57 |
| HNKS | | | | | |

| | Tissue-Specific mRNA Expression | | | | |
|---|---|---|---|---|---|
| | (copies per ng mRNA; avg. ± range for 2 data points per tissue) | | | | |
| | Skele- | | | | |
| | tal | Intes- | Spleen/ | Pla- | |
| Gene Name | muscle | tine | lymph | centa | Testis |
| sbg123493- | 132 ± 21 | 6 ± 2 | 5 ± 10 | 9 ± 4 | 959 ± 80 |
| SLITa | | | | | |
| sbg14936- | 1503 ± 168 | 218 ± 26 | 423 ± 4 | 629 ± 39 | 1765 ± 40 |
| EGFa | | | | | |
| SBh80018- | 6 ± 4 | −3 ± 3 | 2 ± 0 | 0 ± 1 | 5258 ± 259 |
| .cyastin- | | | | | |
| related | | | | | |
| SBh74552- | 4 ± 1 | 3 ± 0 | 10 ± 3 | 5 ± 0 | 5159 ± 907 |
| .trypsinogen | | | | | |
| sbg90060- | 119 ± 15 | 109 ± 4 | 531 ± 12 | 582 ± 8 | 207 ± 13 |
| IGFBP | | | | | |
| sbg97078- | 4 ± 1 | 37 ± 2 | 91 ± 5 | 244 ± 3 | 688 ± 18 |
| ANGIOa | | | | | |
| sbg68091- | 2351 ± 5 | 1646 ± 112 | 486 ± 4 | 3228 ± 327 | 3204 ± 42 |
| CMP | | | | | |
| sbg18525- | 69 ± 7 | 401 ± 62 | 39 ± 3 | 119 ± 17 | 307 ± 1 |
| LRR | | | | | |
| SBh45597- | 45 ± 6 | 36 ± 6 | 49 ± 16 | 57 ± 9 | 219 ± 55 |
| .trypsin | | | | | |
| inhibitor | | | | | |
| sbg34640- | 9611 ± 323 | 31417 ± 619 | 70617 ± 2786 | 203542 ± 4017 | 20011 ± 2747 |
| CALa | | | | | |
| sbg14849- | 125 ± 12 | 348 ± 38 | 662 ± 17 | 1404 ± 138 | 721 ± 69 |
| LO | | | | | |
| SBh35812.- | 8 ± 7 | 5 ± 2 | 15 ± 4 | 20 ± 5 | 136 ± 20 |
| CALGIZ- | | | | | |
| ZARIN | | | | | |
| sbg37967- | 18 ± 1 | 4 ± 3 | 34 ± 10 | 593 ± 62 | 57 ± 5 |
| ECMPa | | | | | |
| sbg15037- | 298 ± 8 | 264 ± 17 | 256 ± 4 | 277 ± 14 | 316 ± 55 |
| SER | | | | | |
| sbg23161- | 79 ± 13 | 809 ± 41 | 1276 ± 17 | 831 ± 22 | 2635 ± 156 |
| EGFa | | | | | |
| sbg82008- | 829 ± 47 | 321 ± 28 | 721 ± 108 | 1037 ± 51 | 670 ± 110 |
| TGFa,b | | | | | |
| sbg2714- | 3783 ± 80 | 173 ± 1 | 211 ± 37 | 5218 ± 240 | 354 ± 39 |
| 2IGBb | | | | | |
| sbg23988- | 0 ± 0 | 3 ± 1 | −2 ± 5 | 4 ± 0 | 780 ± 20 |
| 1TAGL | | | | | |
| sbg248602- | 80 ± 17 | 385 ± 18 | 730 ± 43 | 15644 ± 309 | 921 ± 9 |
| CHP | | | | | |
| sbg219473- | 74 ± 18 | 98 ± 1 | 1121 ± 12 | 10 ± 6 | 2813 ± 148 |
| HNKS | | | | | |
| Quantitative, tissue-specific, mRNA expression patterns of the genes were measured using SYBR- Green Quantitative PCR (Applied Biosystems, Foster City, CA; see Schmittgen T.D. et al., Analytical Biochemistry | | | | | |
| # 285:194-204, 2000) and human cDNAs prepared from various human tissues. Gene-specific PCR primers were designed using the first nucleic acid sequence listed in the Sequence List for each gene. Results are | | | | | |
| # presented as the number of copies of each specific gene's mRNA detected in 1 ng mRNA pool from each tissue. Two replicate mRNA measurements were made from each tissue RNA. | | | | | |

TABLE V
Additional diseases based on mRNA expression in specific tissues

| Tissue | |
|---|---|
| Expression | Additional Diseases |
| Brain | Neurological and psychiatric diseases, including Alzheimers, parasupranuclear palsey, |
| | Huntington's disease, myotonic dystrophy, anorexia, depression, schizophrenia, |
| | headache, amnesias, anxiety disorders, sleep disorders, multiple sclerosis |
| Heart | Cardiovascular diseases, including congestive heart failure, dilated cardiomyopathy, |
| | cardiac arrhythmias, Hodgson's Disease, myocardial infarction, cardiac arrhythmias |
| Lung | Respiratory diseases, including asthma, Chronic Obstructive Pulmonary Disease, cystic |
| | fibrosis, acute bronchitis, adult respiratory distress syndrome |
| Liver | Dyslipidemia, hypercholesterolemia, hypertriglyceridemia, cirrhosis, hepatic |
| | encephalopathy, fatty hepatocirrhosis, viral and nonviral hepatitis, Type II Diabetes |
| | Mellitis, impaired glucose tolerance |
| Kidney | Renal diseases, including acute and chronic renal failure, acute tubular necrosis, |
| | cystinuria, Fanconi's Syndrome, glomerulonephritis, renal cell carcinoma, renovascular |
| | hypertension |
| Skeletal | Eulenburg's Disease, hypoglycemia, obesity, tendinitis, periodic paralyses, malignant |
| muscle | hyperthermia, paramyotonia congenita, myotonia congenita |
| Intestine | Gastrointestinal diseases, including Myotonia congenita, Ileus, Intestinal Obstruction, |
| | Tropical Sprue, Pseudomembranous Enterocolitis |
| Spleen/lymph | Lymphangiectasia, hypersplenism, angiomas, ankylosing spondylitis, Hodgkin's Disease, |
| | macroglobulinemia, malignant lymphomas, rheumatoid arthritis |
| Placenta | Choriocarcinoma, hydatidiform mole, placenta previa |
| Testis | Testicular cancer, male reproductive diseases, including low testosterone and male |
| | infertility |
| Pancreas | Diabetic ketoacidosis, Type 1 & 2 diabetes, obesity, impaired glucose tolerance |

What is claimed is:

1. An isolated polypeptide selected from the group consisting of:
(a) an isolated polypeptide encoded by a polynucleotide comprising a sequence set forth in Table I;
(b) an isolated polypeptide comprising a polypeptide sequence set forth in Table I; and
(c) a polypeptide sequence of a gene set forth in Table I.

2. An isolated polynucleotide selected from the group consisting of:
(a) an isolated polynucleotide comprising a polynucleotide sequence set forth in Table I;
(b) an isolated polynucleotide of a gene set forth in Table I;
(c) an isolated polynucleotide comprising a polynucleotide sequence encoding a polypeptide set forth in Table I;
(d) an isolated polynucleotide encoding a polypeptide set forth in Table I;
(e) a polynucleotide which is an RNA equivalent of the polynucleotide of (a) to (d);
or a polynucleotide sequence complementary to said isolated polynucleotide.

3. An expression vector comprising a polynucleotide capable of producing a polypeptide of claim 1 when said expression vector is present in a compatible host cell.

4. A process for producing a recombinant host cell which comprises the step of introducing an expression vector comprising a polynucleotide capable of producing a polypeptide of claim 1 into a cell such that the host cell, under appropriate culture conditions, produces said polypeptide.

5. A recombinant host cell produced by the process of claim 4.

6. A membrane of a recombinant host cell of claim 5 expressing said polypeptide.

7. A process for producing a polypeptide which comprises culturing a host cell of claim 5 under conditions sufficient for the production of said polypeptide and recovering said polypeptide from the culture.


SEQUENCE LISTING (66 sequences)

SEQ ID NO: 1 - DNA, 771 bp, Homo sapiens
atgtcccttgcttcaggccctggccctgggtggttactcttttcctttggaatggggctggtatcagggtcaaagtgtccaaataattgtctgtgtcaagcccaagaagtaatctgcacagggaagcagttaaccgaatacccccttgacatacccctgaacacccggaggctgttcctgaacgagaacagaatcactagtttgccagcaatgcatctaggactcctcagtgaccttgtttatttggactgtcagaacaaccggattcgagaggtgatggattataccttcatcggggtcttcaaactcatctaccttgacctcagctccaacaacctaacctcgatctccccattcactttctcggtgctcagcaacctggtgcagctgaacattgccaacaaccctcacctgttatcgcttcacaagttcacctttgccaacaccacctctttgaggtacctggacctcagaaataccggcttgcagaccctggacagtgctgccttataccacctcactactctggagaccctgtttctgagtggaaacccctggaagtgcaactgctctttcctggacttcgccatcttcttaatagtgttccatatggacccctcaggtgagggcttgattgggtgtggggaagaggatgtgattgaagtggctccagaaaaggtgaactcaaaagatggtcagaatgggagaaaaagttgggtgaagctgattgaatgcattcttattactctgcagggcccacccttgggttga

SEQ ID NO: 2 - DNA, 2694 bp, Homo sapiens
atgggctcggggcgcgtacccgggctctgcctgcttgtcctgctggtccacgcccgcgccgcccagtacagcaaagccgcgcaagatgtggatgagtgtgtggaggggactgacaactgccacatcgatgctatctgccagaacaccccgaggtcatacaagtgcatctgcaagtctggctacacaggggacggcaaacactgcaaagacgtggatgagtgcgagcgagaggataatgcaggttgtgtgcatgactgtgtcaacatccctggcaattaccggtgtacctgctatgatggattccacctggcacatgacggacacaactgtctggatgtggacgagtgtgccgagggcaacggcggctgtcagcagagctgtgtcaacatgatgggcagctatgagtgccactgccgggaaggcttcttcctcagcgacaaccagcatacctgtatccagcggccagaagaaggaatgaattgcatgaacaagaaccacggctgtgcccacatttgccgggagacacccaaggggggtattgcctgtgaatgccgtcctggctttgagcttaccaagaaccaacgggactgtaaatgtgagataattgggatggcagtgacatgcaactatggtaacggcggctgccagcacacgtgtgatgacacagagcagggtccccggtgcggctgccatatcaagtttgtgctccataccgacgggaagacatgcatcgagacctgtgctgtcaacaacgggggctgtgacagtaagtgccatgatgcagcgactggtgtccactgcacctgccctgtgggcttcatgctgcagccagacaggaagacgtgcaaagatatagatgagtgccgcttaaacaacgggggctgtgaccatatttgccgcaacacagtgggcagcttcgaatgcagttgcaagaaaggctataagcttctcatcaatgagaggaactgccaggatatagacgagtgttcctttgatcgaacctgtgaccacatatgtgtcaacacaccaggaagcttccagtgtctctgccatcgtggctacctgttgtatggtatcacccactgtggggatgtggatgaatgcagcatcaaccggggaggttgccgctttggctgcatcaacactcctggcagctaccagtgtacctgcccagcaggccagggtcggctgcactggaatggcaaagattgcacagagccactgaagtgtcagggcagtcctggggcctcgaaagccatgctcagctgcaaccggtctggcaagaaggacacctgtgccctgacctgtccctccagggcccgatttttgccagagtctgagaatggcttcacggtgagctgtgggacccccagccccagggctgctccagcccgagctggccacaatgggaacagcaccaactccaaccactgccatgaggctgcagtgctgtccattaaacaacgggcctccttcaagatcaaggatgccaaatgccgtttgcacctgcgaaacaaaggcaaaacagaggaggctggcagaatcacagggccaggtggtgccccctgctctgaatgccaggtcaccttcatccaccttaagtgtgactcctctcggaagggcaagggccgacgggcccggacccctccaggcaaagaggtcacaaggctcaccctggaactggaggcagagcaactctttctcctccctgatacacacggccatccaccaccagccagctgtgggctgccctgcctccgacagcgaatggaacggcggctgaaaggatccctgaagatgctcagaaagtccatcaaccaggaccgcttcctgctgcgcctggcaggccttgattatgagctggcccacaagccgggcctggtagccggggagcgagcagagccgatggagtcctgtaggcccgggcagcaccgtgctgggaccaagtgtgtccagtgctccccagggcactactacaacaccagcatccaccgctgtattcgctgtgccatgggctcctatcagcccgacttccgtcagaacttctgcagccgctgtccaggaaacacaagcacagactttgatggctctaccagtgtggcccaatgcaagaatcgtcagtgtggtggggagctgggtgagttcactggctatattgagtcccccaactacccgggcaactacccagctggtgtggagtgcatctggaacatcaaccccccacccaagcgcaagatccttatcgtggtaccagagatcttcctgccatctgaggatgagtgtggggacgtcctcgtcatgagaaagaactcatccccatcctccattaccacttatgagacctgccagacctacgagcgtcccattgccttcactgcccgttccaggaagctctggatcaacttcaagacaagcgaggccaacagcgcccgtggcttccagattccctatgttacctatgatgaggactatgagcagctggtagaagacattgtgcgagatggccggctctatgcctctgaaaaccaccaggagattttaaaggacaagaagctcatcaaggccttctttgaggtgctagcccacccccagaactacttcaagtacacagagaaacacaaggagatgctgccaaaatccttcatcaagctgctccgctccaaagtttccagcttcctgaggccctacaaatag

SEQ ID NO: 3 - DNA, 2982 bp, Homo sapiens
atgggctcggggcgcgtacccgggctctgcctgcttgtcctgctggtccacgcccgcgccgcccagtacagcaaagccgcgcaggatgtggatgagtgtgtggaggggactgacaactgccacatcgatgctatctgccagaacaccccgaggtcatacaagtgcatctgcaagtctggctacacaggggacggcaaacactgcaaagacgtggatgagtgcgagcgagaggataatgcaggttgtgtgcatgactgtgtcaacatccctggcaattaccggtgtacctgctatgatggattccacctggcacatgacggacacaactgtctggatgtggacgagtgtgccgagggcaacggcggctgtcagcagagctgtgtcaacatgatgggcagctatgagtgccactgccgggaaggcttcttcctcagcgacaaccagcatacctgtatccagcggccagaagaaggaatgaattgcatgaacaagaaccacggctgtgcccacatttgccgggagacacccaaggggggtattgcctgtgaatgccgtcctggctttgagcttaccaagaaccaacgggactgtaaattgacatgcaactatggtaacggcggctgccagcacacgtgtgatgacacagagcagggtccccggtgcggctgccatatcaagtttgtgctccataccgacgggaagacatgcatcgagacctgtgctgtcaacaacgggggctgtgacagtaagtgccatgatgcagcgactggtgtccactgcacctgccctgtgggcttcatgctgcagccagacaggaagacgtgcaaagatatagatgagtgccgcttaaacaacgggggctgtgaccatatttgccgcaacacagtgggcagcttcgaatgcagttgcaagaaaggctataagcttctcatcaatgagaggaactgccaggatatagacgagtgttcctttgatcgaacctgtgaccacatatgtgtcaacacaccaggaagcttccagtgtctctgccatcgtggctacctgttgtatggtatcacccactgtggggatgtggatgaatgcagcatcaaccggggaggttgccgctttggctgcatcaacactcctggcagctaccagtgtacctgcccagcaggccagggtcggctgcactggaatggcaaagattgcacagagccactgaagtgtcagggcagtcctggggcctcgaaagccatgctcagctgcaaccggtctggcaagaaggacacctgtgccctgacctgtccctccagggcccgatttttgccagagtctgagaatggcttcacggtgagctgtgggacccccagccccagggctgctccagcccgagctggccacaatgggaacagcaccaactccaaccactgccatgaggctgcagtgctgtccattaaacaacgggcctccttcaagatcaaggatgccaaatgccgtttgcacctgcgaaacaaaggcaaaacagaggaggctggcagaatcacagggccaggtggtgccccctgctctgaatgccaggtcaccttcatccaccttaagtgtgactcctctcggaagggcaagggccgacgggcccggacccctccaggcaaagaggtcacaaggctcaccctggaactggaggcagaggtcagagccgaagaaaccacagccagctgtgggctgccctgcctccgacagcgaatggaacggcggctgaaaggatccctgaagatgctcagaaagtccatcaaccaggaccgcttcctgctgcgcctggcaggccttgattatgagctggcccacaagccgggcctggtagccggggagcgagcagagccgatggagtcctgtaggcccgggcagcaccgtgctgggaccaagtgtgtcagctgcccgcagggaacgtattaccacggccagacggagcagtgtgtgccatgcccagcgggcaccttccaggagagagaagggcagctctcctgcgacctttgccctgggagtgatgcccacgggcctcttggagccaccaacgtcaccacgtgtgcaggtcagtgcccacctggccaacactctgtagatgggttcaagccctgtcagccatgcccacgtggcacctaccaacctgaagcaggacggaccctatgcttcccttgtggtgggggcctcaccaccaagcatgaaggggccatttccttccaagactgtgacaccaaagtccagtgctccccagggcactactacaacaccagcatccaccgctgtattcgctgtgccatgggctcctatcagcccgacttccgtcagaacttctgcagccgctgtccaggaaacacaagcacagactttgatggctctaccagtgtggcccaatgcaagaatcgtcagtgtggtggggagctgggtgagttcactggctatattgagtcccccaactacccgggcaactacccagctggtgtggagtgcatctggaacatcaaccccccacccaagcgcaagatccttatcgtggtaccagagatcttcctgccatctgaggatgagtgtggggacgtcctcgtcatgagaaagaactcatccccatcctccattaccacttatgagacctgccagacctacgagcgtcccattgccttcactgcccgttccaggaagctctggatcaacttcaagacaagcgaggccaacagcgcccgtggcttccagattccctatgttacctatgatgaggactatgagcagctggtagaagacattgtgcgagatggccggctctatgcctctgaaaaccaccaggagattttaaaggacaagaagctcatcaaggccttctttgaggtgctagcccacccccagaactacttcaagtacacagagaaacacaaggagatgctgccaaaatccttcatcaagctgctccgctccaaagtttccagcttcctgaggccctacaaatag

SEQ ID NO: 4 - DNA, 417 bp, Homo sapiens
atggtccggctctgccaggccctgctgctgttagtggccactgtggcccttgcatccagaagattccaagcctggggctcaacaaaggtggtgaggacattccaagatatccctcaaaactacgtctatgtgcagcaggcactctggttcgccatgaaggagtataacaaggccagctttagtataacaagttcagctttagggaaagaatacaaattaaaggtgacagatagtttggagtactatattgaggtcaaaattgcccgaacaatttgcaagaaaatttcagaagatgaaaactgtgcatttcaagaggatcccaaaatgcaaaaggtggttttttgtacttttattgttgcatctaaaccatggaaatttgaactcaccatgctgaagaaacaatgcaaagatatgtag

SEQ ID NO: 5 - DNA, 726 bp, Homo sapiens
atgaagtttatcctcctctgggccctcttgaatctgactgttgctttggcctttaatccagattacacagtcagctccactcccccttacttggtctatttgaaatctgactacttgccctgcgctggagtcctgatccacccgctttgggtgatcacagctgcacactgcaatttaccaaagcttcgggtgatattgggggttacaatcccagcagactctaatgaaaagcatctgcaagtgattggctatgagaagatgattcatcatccacacttctcagtcacttctattgatcatgacatcatgctaatcaagctgaaaacagaggctgaactcaatgactatgtgaaattagccaacctgccctaccaaactatctctgaaaataccatgtgctctgtctctacctggagctacaatgtgtgtgatatctacaaagagcccgattcactgcaaactgtgaacatctctgtaatctccaagcctcagtgtcgcgatgcctataaaacctacaacatcacggaaaatatgctgtgtgtgggcattgtgccaggaaggaggcagccctgcaaggaagtttctgctgccccggcaatctgcaatgggatgcttcaaggaatcctgtcttttgcggatggatgtgttttgagagccgatgttggcatctatgccaaaattttttactatataccctggattgaaaatgtaatccaaaataactga

SEQ ID NO: 6 - DNA, 732 bp, Homo sapiens
atgactgagaaatcttggaatttcttgtctatgcttctctttccagttgctttggcctttaatccagattacacagtcagctccactcccccttacttggtctatttgaaatctgactacttgccctgcgctggagtcctgatccacccgctttgggtgatcacagctgcacactgcaatttaccaaagcttcgggtgatattgggggttacaatcccagcagactctaatgaaaagcatctgcaagtgattggctatgagaagatgattcatcatccacacttctcagtcacttctattgatcatgacatcatgctaatcaagctgaaaacagaggctgaactcaatgactatgtgaaattagccaacctgccctaccaaactatctctgaaaataccatgtgctctgtctctacctggagctacaatgtgtgtgatatctacaaagagcccgattcactgcaaactgtgaacatctctgtaatctccaagcctcagtgtcgcgatgcctataaaacctacaacatcacggaaaatatgctgtgtgtgggcattgtgccaggaaggaggcagccctgcaaggaagtttctgctgccccggcaatctgcaatgggatgcttcaaggaatcctgtcttttgcggatggatgtgttttgagagccgatgttggcatctatgccaaaattttttactatataccctggattgaaaatgtaatccaaaataactga

SEQ ID NO: 7 - DNA, 1452 bp, Homo sapiens
atgtacccaggctggcccgggcagggcatgtgggcgagcggacagcgcctgcctgacgaggccttcgagtccctcacccagctgcagcacctctgcgtggctcacaacaagctctcagtggcccctcagtttctgccccggtccctccgtgtcgcggatctggctgccaaccaagtgatggagatcttccccctcacctttggggagaagccggcactcaggtccgtgtacctccacaacaaccagctgagcaacgctggcctgccccccgacgccttccgcggctccgaggccatcgccaccctcagcctctccaacaaccagctcagctacctgccgcccagcctgccgccctcactcgagcggctccacctgcagaacaatctcatctccaaggtgccccgaggagccctgagccgccagactcaactccgtgagctctacctccagcacaaccagctgacagacagtggcctggatgccaccaccttcagcaagctgcatagccttgaatacctggatctctcccacaaccagctgaccacagtgcccgccggcctgccccggaccctggctatcctgcacctgggccgcaaccgcatccggcaggtggaggcggctcggctgcacggggcgcgtggtctgcgctatttgttgctgcagcacaaccagctggggagctcagggctgcccgccggggctctgcggccgctgcggggcctgcacacgctgcacctctatggcaatgggctggaccgcgtgcctccagccctgccccgccgcctgcgtgccctggtgctgccccacaaccacgtggccgcgctgggtgcccgtgacctggtcgccacaccgggcctgacggagcttaacctggcctataaccgcctggccagcgcccgtgtgcaccaccgggccttccgccggttgcgtgccctgcgcagcctcgacctggcagggaatcagctaacccggctgcccatgggcctgcccactggcctgcgcaccctgcagctgcaacgcaaccagctgcggatgctcgagcccgagcctctggccggcctggaccaactgcgggagctcagcctggcgcacaaccggctccgggtcggcgacatcgggccaggcacctggcatgagctccaagccctccagatgctggacctcagccacaatgagctgtcctttgtgcccccggacctgcctgaggccctagaggagctgcacctcgagggcaaccgcatcggccacgtgggccccgaggccttcctcagcacaccccgcctgcgtgccctcttcctcagggccaacaggcttcacatgacgagcatcgcggctgaggccttcctggggctcccaaacctgcgtgtggtggacacggcagggaatccggagcaggtcctgatccggctgcctcccaccaccccacgtgggccacgggcagggggcccctga

SEQ ID NO: 8 - DNA, 1818 bp, Homo sapiens
atggcagagtcagggctggccatggaggggatgctccagtcaccatggcgaccctgcgcccagcctggagacacgctgaccctccctcccccgcagtggccgagcctgctgctgctcctgctgttgccggggcccccgcccgtcgccggcttggaagacgctgccttcccccacctgggggagagcttgcagcccctgccccgggcctgtcccctgcgctgctcctgcccccgagtcgacactgtggactgtgatggcttggaccttcgagtgttcccggacaacatcaccagagccgctcagcacctctccctgcagaacaaccagctccaggaactcccctacaatgagctgtcccgcctcagtggcctgcgaaccctcaacctccacaacaacctcatctcctccgaaggcctgcctgacgaggccttcgagtccctcacccagctgcagcacctctgcgtggctcacaacaagctctcagtggcccctcagtttctgccccggtccctccgtgtcgcggatctggctgccaaccaagtgatggagatcttccccctcacctttggggagaagccggcactcaggtccgtgtacctccacaacaaccagctgagcaacgctggcctgccccccgacgccttccgcggctccgaggccatcgccaccctcagcctctccaacaaccagctcagctacctgccgcccagcctgccgccctcactcgagcggctccacctgcagaacaatctcatctccaaggtgccccgaggagccctgagccgccagactcaactccgtgagctctacctccagcacaaccagctgacagacagtggcctggatgccaccaccttcagcaagctgcatagccttgaatacctggatctctcccacaaccagctgaccacagtgcccgccggcctgccccggaccctggctatcctgcacctgggccgcaaccgcatccggcaggtggaggcggctcggctgcacggggcgcgtggtctgcgctatttgttgctgcagcacaaccagctggggagctcagggctgcccgccggggctctgcggccgctgcggggcctgcacacgctgcacctctatggcaatgggctggaccgcgtgcctccagccctgccccgccgcctgcgtgccctggtgctgccccacaaccacgtggccgcgctgggtgcccgtgacctggtcgccacaccgggcctgacggagcttaacctggcctataaccgcctggccagcgcccgtgtgcaccaccgggccttccgccggttgcgtgccctgcgcagcctcgacctggcagggaatcagctaacccggctgcccatgggcctgcccactggcctgcgcaccctgcagctgcaacgcaaccagctgcggatgctcgagcccgagcctctggccggcctggaccaactgcgggagctcagcctggcgcacaaccggctccgggtcggcgacatcgggccaggcacctggcatgagctccaagccctccagatgctggacctcagccacaatgagctgtcctttgtgcccccggacctgcctgaggccctagaggagctgcacctcgagggcaaccgcatcggccacgtgggccccgaggccttcctcagcacaccccgcctgcgtgccctcttcctcagggccaacaggcttcacatgacgagcatcgcggctgaggccttcctggggctcccaaacctgcgtgtggtggacacggcagggaatccggagcaggtcctgatccggctgcctcccaccaccccacgtgggccacgggcagggggcccctga

SEQ ID NO: 9 - DNA, 3150 bp, Homo sapiens
atggtaactcgtgaactgtttttccttttttccccccagttcttcagccttaacctaaggtctcatactcggagcactatgacatcgccccagctagagtggactctgcagacccttctggagcagctgaacgaggatgaattaaagagtttcaaatcccttttatgggcttttcccctcgaagacgtgctacagaagaccccatggtctgaggtggaagaggctgatggcaagaaactggcagaaattctggtcaacacctcctcagaaaattggataaggaatgcgactgtgaacatcttggaagagatgaatctcacggaattgtgtaagatggcaaaggctgagatgatggaggacggacaggtgcaagaaatagataatcctgagctgggagatgcagaagaagactcggagttagcaaagccaggtgaaaaggaaggatggagaaattcaatggagaaacagtctttggtctggaagaacaccttttggcaaggagacattgacaatttccatgacgacgtcactctgagaaaccaacggttcattccattcttgaatcccagaacacccaggaagctaacaccttacacggtggtgctgcacggccccgcaggcgtggggaaaaccacgctggccaaaaagtgtatgctggactggacagactgcaacctcagcccgacgctcagatacgcgttctacctcagctgcaaggagctcagccgcatgggcccctgcagttttgcagagctgatctccaaagactggcctgaattgcaggatgacattccaagcatcctagcccaagcacagagaatcctgttcgtggtcgatggccttgatgagctgaaagtcccacctggggcgctgatccaggacatctgcggggactgggagaagaagaagccggtgcccgtcctcctggggagtttgctgaagaggaagatgttacccagggcagccttgctggtcaccacgcggcccagggcactgagggacctccagctcctggcgcagcagccgatctacgtaagggtggagggcttcctggaggaggacaggagggcctatttcctgagacactttggagacgaggaccaagccatgcgtgcctttgagctaatgaggagcaacgcggccctgttccagctgggctcggcccccgcggtgtgctggattgtgtgcacgactctgaagctgcagatggagaagggggaggacccggtccccacctgcctcacccgcacggggctgttcctgcgtttcctctgcagccggttcccgcagggcgcacagctgcggggcgcgctgcggacgctgagcctcctggccgcgcagggcctgtgggcgcagatgtccgtgttccaccgagaggacctggaaaggctcggggtgcaggagtccgacctccgtctgttcctggacggagacatcctccgccaggacagagtctccaaaggctgctactccttcatccacctcagcttccagcagtttctcactgccctgttctacgccctggagaaggaggagggggaggacagggacggccacgcctgggacatcggggacgtacagaagctgctttccggagaagaaagactcaagaaccccgacctgattcaagtaggacacttcttattcggcctcgctaacgagaagagagccaaggagttggaggccacttttggctgccggatgtcaccggacatcaaacaggaattgctgcaatgcaaagcacatcttcatgcaaataagcccttatccgtgaccgacctgaaggaggtcttgggctgcctgtatgagtctcaggaggaggagctggcgaaggtggtggtggccccgttcaaggaaatttctattcacctgacaaatacttctgaagtgatgcattgttccttcagcctgaagcattgtcaagacttgcagaaactctcactgcaggtagcaaagggggtgttcctggagaattacatggattttgaactggacattgaatttgaaagctcaaacagcaacctcaagtttctggaagtgaaacaaagcttcctgagtgactcttctgtgcggattctttgtgaccacgtaacccgtagcacctgtcatctgcagaaagtggagattaaaaacgtcacccctgacaccgcgtaccgggacttctgtcttgctttcattgggaagaagaccctcacgcacctgaccctggcagggcacatcgagtgggaacgcacgatgatgctgatgctgtgtgacctgctcagaaatcataaatgcaacctgcagtacctgaggttgggaggtcactgtgccaccccggagcagtgggctgaattcttctatgtcctcaaagccaaccagtccctgaagcacctgcgtctctcagccaatgtgctcctggatgagggtgccatgttgctgtacaagaccatgacacgcccaaaacacttcctgcagatgttgtcgttggaaaactgtcgtcttacagaagccagttgcaaggaccttgctgctgtcttggttgtcagcaagaagctgacacacctgtgcttggccaagaaccccattggggatacaggggtgaagtttctgtgtgagggcttgagttaccctgattgtaaactgcagaccttggtgttggtgtcttgttccgctaccactcagcagtgggctgatctctccttggcccttgaagtcaaccagtccctgacgtgcgtaaacctctccgacaatgagcttctggatgagggtgctaagttgctgtacacaactttgagacaccccaagtgctttctgcagaggttgtcgttggaaaactgtcaccttacagaagccaattgcaaggaccttgctgctgtgttggttgtcagccgggagctgacacacctgtgcttggccaagaaccccattgggaatacaggggtgaagtttctgtgtgagggcttgaggtaccccgagtgtaaactgcagaccttggtgttacagcaatgcagcataaccaagcttggctgtagatatctctcagaggcgctccaagaagcctgcagcctcacaaacctggacttgagtatcaaccagatagctcgtggattgtggattctctgtcaggcattagagaatccaaactgtaacctaaaacacctacggtag

SEQ ID NO: 10 - DNA, 3189 bp, Homo sapiens
atggtgtcttcggcgcagatgggcttcaacctgcaggctctcctggagcagctcagccaggatgagttgagcaagttcaagtatctgatcacgaccttctccctggcacacgagctccagaagatcccccacaaggaggtagacaaggctgatgggaagcaactggtagaaatcctcaccacccattgtgacagctactgggtggagatggcgagcctccaggtctttgaaaagatgcaccgaatggatctgtctgagagagcaaaggatgaagtcagagaagcagctttgaaatcctttaataaaaggaagcctctatcattagggataacacggaaagaacgaccacctctagacgtggacgaaatgctggagcgcttcaaaacagaagcacaagcgtttacagaaacgaaaggaaatgtcatctgcctgggtaaagaagtctttaaaggaaaaaagccagacaaagacaataggtgcaggtatatattgaagacgaagttccgggagatgtggaagagctggcctggagatagcaaagaggtccaggttatggctgagagatacaagatgctgatcccattcagcaaccccagggtgcttcccgggcccttctcatacacggtggtgctgtatggtcctgcaggccttgggaaaaccacgctggcccagaaactaatgctagactgggcagaggacaacctcatccacaaattcaaatatgcgttctacctcagctgcagggagctcagccgcctgggcccgtgcagttttgcagagctggtcttcagggactggcctgaattgcaggatgacattccacacatcctagcccaagcacggaaaatcttgttcgtgattgacggctttgatgagctgggagccgcacctggggcgctgatcgaggacatctgcggggactgggagaagaagaagccggtgcccgtcctcctggggagtttgctgaacagggtgatgttacccaaggccgccctgctggtcaccacgcggcccagggccctgagggacctccggatcctggcggaggagccgatctacataagggtggagggcttcctggaggaggacaggagggcctatttcctgagacactttggagacgaggaccaagccatgcgtgcctttgagctaatgaggagcaacgcggccctgttccagctgggctcggcccccgcggtgtgctggatcgtgtgcacgactctgaagctgcagatggagaagggggaggacccggtccccacctgcctcacccgcacggggctgttcctgcgtttcctctgcagccggttcccgcagggcgcacagctgcggggcgcgctgcggacgctgagcctcctggccgcgcagggcctgtgggcgcagacgtccgtgcttcaccgagaggatctggaaaggctcggggtgcaggagtccgacctccgtctgttcctggacggagacatcctccgccaggacagagtctccaaaggctgctactccttcatccacctcagcttccagcagtttctcactgccctgttctacaccctggagaaggaggaggaagaggatagggacggccacacctgggacattggggacgtacagaagctgctttccggagtagaaagactcaggaaccccgacctgatccaagcaggctactactcctttggcctcgctaacgagaagagagccaaggagttggaggccacttttggctgccggatgtcaccggacatcaaacaggaattgctgcgatgcgacataagttgtaagggtggacattcaacggtgacagacctgcaggagctcctcggctgtctgtacgagtctcaggaggaggagctggtgaaggaggtgatggctcagttcaaagaaatatccctgcacttaaatgcagtagacgttgtgccatcttcattctgcgtcaagcactgtcgaaacctgcagaaaatgtcactgcaggtaataaaggagaatctcccggagaatgtcactgcgtctgaatcagacgccgaggttgagagatcccaggatgatcagcacatgcttcctttctggacggacctttgttccatatttggatcaaataaggatctgatgggtctagcaatcaatgatagctttctcagtgcctccctagtaaggatcctgtgtgaacaaatagcctctgacacctgtcatctccagagagtggtgttcaaaaacatttccccagctgatgctcatcggaacctctgcctagctcttcgaggtcacaagactgtaacgtatctgacccttcaaggcaatgaccaggatgatatgtttcccgcattgtgtgaggtcttgagacatccagaatgtaacctgcgatatctcgggttggtgtcttgttccgctaccactcagcagtgggctgatctctccttggcccttgaagtcaaccagtccctgacgtgcgtaaacctctccgacaatgagcttctggatgagggtgctaagttgctgtacacaactttgagacaccccaagtgctttctgcagaggttgtcgttggaaaactgtcaccttacagaagccaattgcaaggaccttgctgctgtgttggttgtcagccgggagctgacacacctgtgcttggccaagaaccccattgggaatacaggggtgaagtttctgtgtgagggcttgaggtaccccgagtgtaaactgcagaccttggtgctttggaactgcgacataactagcgatggctgctgcgatctcacaaagcttctccaagaaaaatcaagcctgttgtgtttggatctggggctgaatcacataggagttaagggaatgaagttcctgtgtgaggctttgaggaaaccactgtgcaacttgagatgtctgtggttgtggggatgttccatccctccgttcagttgtgaagacctctgctctgccctcagctgcaaccagagcctcgtcactctggacctgggtcagaatcccttggggtctagtggagtgaagatgctgtttgaaaccttgacatgttccagtggcaccctccggacactcaggttgaaaatcgatgactttaatgatgaactcaataagctgctggaagaaatagaagaaaaaaacccacaactgattattgatactgagaaacatcatccctgggcagaaaggccttcttctcatgacttcatgatctga

SEQ ID NO: 11 - DNA, 1062 bp, Homo sapiens
atgacaatttttcatcccattacttcatccattggccagcctggttgtgggcccaaatgcaaagagactccactagagctggtgtttgtgatcgacagctcagaaagcgtggggccagagaactttcagatcattaaaaattttgtgaagactatggctgaccgggttgctctggaccttgccacggcccgcataggcataatcaactatagccataaggtggagaaggtggctaatttgaagcagttctccagcaaggatgacttcaagttggctgtggacaacatgcagtatctgggggaaggcacatacacagccactgctctgcaagcagccaacgacatgtttgaagatgcaaggccaggtgtaaaaaaagtggccttggtcatcactgatggacagacagattctcgtgataaagagaaactgacagaggtggtgaagaatgccagtgacaccaatgtggagatatttgtgataggggtggtgaagaaaaatgatcccaactttgaaatattccacaaagaaatgaatctaattgctactgacccagagcatgtttaccagtttgatgatttctttaccctgcaagacaccctgaagcaaaaattgtttcaaaaaatttgtgaggattttgattcctatctcgttcaaatttttggttcatcgtcacctcaacctggatttgggatgtcaggggaagaactcagtgaatctactccagagcctcaaaaagaaatttctgagtcattgagtgtcaccagagaccaggatgaagatgataaggctccagagccaacgtgggctgatgatctgcctgccactacctcatctgaggccaccaccacccccaggccactgctcagcacccctgtggatggggcagaggatcctagatgtttggaagccttgaagcctggaaactgtggtgaatatgtggttcgatggtattatgacaaacaggtcaactcttgtgcccgattttggttcagtggctgtaatggctcaggaaatagattcaacagtgaaaaggaatgtcaagaaacctgcattcaaggatga

SEQ ID NO: 12 - DNA, 1347 bp, Homo sapiens
atgcatgaggtaattgaatctgactatgaggggagagataaaaccttgtcctgccttgtggtgggtgtgtgtgactactccactcggatgcttggtagaaatgatcacactgctgttactggccaacaaggagcctggtcagagtctgcctccttggaccacagtcccatcctcagtttcctgccccaggaattcccagcagatcgagatggttccttggctctccatagcacttatgaaagtcttcgtttgtctgcttcttcctggactgtgaatcctttgaggggtataaatatgatgccttcatcattggcaccaagtagccaaggttgtgggcccaaatgcaaagagactccactagagctggtgtttgtgatcgacagctcagaaagcgtggggccagagaactttcagatcattaaaaattttgtgaagactatggctgaccgggttgctctggaccttgccacggcccgcataggcataatcaactatagccataaggtggagaaggtggctaatttgaagcagttctccagcaaggatgacttcaagttggctgtggacaacatgcagtatctgggggaaggcacatacacagccactgctctgcaagcagccaacgacatgtttgaagatgcaaggccaggtgtaaaaaaagtggccttggtcatcactgatggacagacagattctcgtgataaagagaaactgacagaggtggtgaagaatgccagtgacaccaatgtggagatatttgtgataggggtggtgaagaaaaatgatcccaactttgaaatattccacaaagaaatgaatctaattgctactgacccagagcatgtttaccagtttgatgatttctttaccctgcaagacaccctgaagcaaaaattgtttcaaaaaatttgtgaggattttgattcctatctcgttcaaatttttggttcatcgtcacctcaacctggatttgggatgtcaggggaagaactcagtgaatctactccagagcctcaaaaagaaatttctgagtcattgagtgtcaccagagaccaggatgaagatgataaggctccagagccaacgtgggctgatgatctgcctgccactacctcatctgaggccaccaccacccccaggccactgctcagcacccctgtggatggggcagaggatcctagatgtttggaagccttgaagcctggaaactgtggtgaatatgtggttcgatggtattatgacaaacaggtcaactcttgtgcccgattttggttcagtggctgtaatggctcaggaaatagattcaacagtgaaaaggaatgtcaagaaacctgcattcaaggatga

SEQ ID NO: 13 - DNA, 1482 bp, Homo sapiens
atgctgcccgccgcccccagcgggtgcccgcagctgtgccggtgcgaggggcggctgctgtactgcgaggcgctcaacctcaccgaggcgccccacaacctgtccggcctgctgggcttgtccctgcgctacaacagcctctcggagctgcgcgccggccagttcacggggttaatgcagctcacgtggctctatctggatcacaatcacatctgctccgtgcagggggacgcctttcagaaactgcgccgagttaaggaactcacgctgagttccaaccagatcacccaactgcccaacaccaccttccggcccatgcccaacctgcgcagcgtggacctctcgtacaacaagctgcaggcgctcgcgcccgacctcttccacgggctgcggaagctcaccacgctgcatatgcgggccaacgccatccagtttgtgcccgtgcgcatcttccaggactgccgcagcctcaagtttctcgacatcggatacaatcagctcaagagtctggcgcgcaactctttcgccggcttgtttaagctcaccgagctgcacctcgagcacaacgacttggtcaaggtgaacttcgcccacttcccgcgcctcatctccctgcactcgctctgcctgcggaggaacaaggtggccattgtggtcagctcgctggactgggtttggaacctggagaaaatggacttgtcgggcaacgagatcgagtacatggagccccatgtgttcgagaccgtgccgcacctgcagtccctgcagctggactccaaccgcctcacctacatcgagccccggatcctcaactcttggaagtccctgacaagcatcaccctggccgggaacctgtgggattgcgggcgcaacgtgtgtgccctagcctcgtggctcaacaacttccaggggcgctacgatggcaacttgcagtgcgccagcccggagtacgcacagggcgaggacgtcctggacgccgtgtacgccttccacctgtgcgaggatggggccgagcccaccagcgggcacctgctctcggccgtcaccaaccgcagtgatctggggccccctgcaaggcgggccaccacggcctcgcggaccgggggggaggggcagcacgacggcacattcaagcctgccaccgggggttttccagccggggagcacgcgaagaaccccgtgcagatccacaaggtggtcacgggcaccatggccttcattttttctttcctcatggtggtcctggtgctctacgtgtcctggaagtgtttcccagccagcctcaggcagctcagacagtgctttgtcacgcagcgcaggaagcaaaagcagaaacagaccatgcatcagatggctgccatgtctgcccaggaatactacgttgattacaaaccgaaccacattgagggagccctggtgatcatcaacgagtatggctcgtgtacctgccaccagcagcccgcgagggaatgcgaggtgtga

SEQ ID NO: 14 - DNA, 1647 bp, Homo sapiens
atgcccgccctacgtccactcctgccgctcctgctcctcctccggctgacctcgggggctggcttgctgccagggctggggagccacccgggcgtgtgccccaaccagctcagccccaacctgtgggtggacgcccagagcacctgtgagcgcgagtgtagcagggaccaggactgtgcggctgctgagaagtgctgcatcaacgtgtgtggactgcacagctgcgtggcagcacgcttccccggcagcccagctgcgccgacgacagcggcctcctgcgagggctttgtgtgcccacagcagggctcggactgcgacatctgggacgggcagcccgtgtgccgctgccgcgaccgctgtgagaaggagcccagcttcacctgcgcctcggacggcctcacctactacaaccgctgctatatggacgccgaggcctgcctgcggggcctgcacctccacatcgtgccctgcaagcacgtgctcagctggccgcccagcagcccggggccgccggagaccactgcccgccccacacctggggccgcgcccgtgcctcctgccctgtacagcagcccctccccacaggcggtgcaggttgggggtacggccagcctccactgcgacgtcagcggccgcccgccgcctgctgtgacctgggagaagcagagtcaccagcgagagaacctgatcatgcgccctgatcagatgtatggcaacgtggtggtcaccagcatcgggcagctggtgctctacaacgcgcggcccgaagacgccggcctgtacacctgcaccgcgcgcaacgctgctgggctgctgcgggctgacttcccactctctgtggtccagcgagagccggccagggacgcagcccccagcatcccagccccggccgagtgcctgccggatgtgcaggcctgcacgggccccacttccccacaccttgtcctctggcactacgacccgcagcggggcggctgcatgaccttcccggcccgtggctgtgatggggcggcccgcggctttgagacctacgaggcatgccagcaggcctgtgcccgcggccccggcgacgcctgcgtgctgcctgccgtgcagggcccctgccggggctgggagccgcgctgggcctacagcccgctgctgcagcagtgccatcccttcgtgtacggtggctgcgagggcaacggcaacaacttccacagccgcgagagctgcgaggatgcctgccccgtgccgcgcacaccgccctgccgcgcctgccgcctccggagcaagctggcgctgagcctgtgccgcagcgacttcgccatcgtggggcggctcacggaggtgctggaggagcccgaggccgccggcggcatcgcccgcgtggcgctcgaggacgtgctcaaggatgacaagatgggcctcaagttcttgggcaccaagtacctggaggtgacgctgagtggcatggactgggcctgcccctgccccaacatgacggcgggcgacgggccgctggtcatcatgggtgaggtgcgcgatggcgtggccgtgctggacgccggcagctacgtccgcgccgccagcgagaagcgcgtcaagaagatcttggagctgctggagaagcaggcctgcgagctgctcaaccgcttccaggactag

SEQ ID NO: 15 - DNA, 861 bp, Homo sapiens
atggcctttgtggcaatcgtggtgtccaactttggcctctcaggtcagcctcatgggggcttcaacagccaggaccaaaatgaccaaggcccctccgtccctgtgtccctgcttgacagaaccaccggaggagggagcgccctgtgcttcctcgcagggatcgactacaagaccaccaccatcctgctggacggccggcgcgtgaagctggagctctgggacacgtcgggccagggccggttctgcaccatcttcaggtcctactccaggggcgctcaggggatcctcttggtgtatgacatcaccaaccgctggtcctttgacggcatcgaccgctggatcaaggagatcgatgagcatgcacccggagtcccccggatcttggttggaaaccggctgcacctggccttcaagcggcaggtcccgacggagcaggcccgcgcgtacgcagagaagaactgcatgaccttctttgaggtcagccccctgtgcaacttcaacgtcatcgagtccttcacggagctatcccgcatcgtgctcatgcggcacggcatggagaagatctggaggcccaaccgagtgttcagcctgcaggacctctgctgccgggccatcgtctcctgcacccccgtgcacctcatcgacaagcttccactgcccgtcaccatcaagagccacctcaagtccttctcgatggccaacggcatgaacgcggtcatgatgcacggccgttcctactccctggccagcggggccgggggcggcggcagcaagggcaacagcctcaagaggtccaagtccatccgtccaccccagagccccccccagaactgctcgcggagtaactgcaagatctcctag

SEQ ID NO: 16 - DNA, 519 bp, Homo sapiens
atgggcatccccatcccaatcatccctcaccacccccaggctcgggtcgcgtccccccaggctttgatggacaagtggccatggaaagcatcctcagctgccccagggttctgccatcacccatccactaaatggtccagggaccctgggaggcaccctgagtctccacatcggggtggctctggggtacacagacgaagcagagagccggcaccccatcctgcgtctgaggaatccagctttccctggctggaagacccggtcatgaagtatgtgggaaagggtggttataactgcactctctccaagacggagttcctaagcttcatgaatgcagaactggctgccttcacaaagaaccagaaggaccccggggtccttcaccgcatgatgaagaaactgggcaccaacaatgacgggcagctagatttctcagaatttcttaatctgattggcggcctagctatggcttgccatgactccttcctcaaggctgtcccttcccagaagcggacctga

SEQ ID NO: 17 - DNA, 312 bp, Homo sapiens
ctgcaaaaatctccagccctgcagagactgagcatcgagtccctgatttctcttttccagaagtatgtgggaaagggtggttataactgcactctctccaagacggagttcctaagcttcatgaatgcagaactggctgccttcacaaagaaccagaaggaccccggggtccttcaccgcatgatgaagaaactgggcaccaacaatgacgggcagctagatttctcagaatttcttaatctgattggcggcctagctatggcttgccatgactccttcctcaaggctgtcccttcccagaagcggacctga

SEQ ID NO: 18 - DNA, 2262 bp, Homo sapiens
atgcgacctgtcagtgtctggcagtggagcccctgggggctgctgctgtgcctgctgtgcagttcgtgcttggggtctccgtccccttccacgggccctgagaagaaggccgggagccaggggcttcggttccggctggctggcttccccaggaagccctacgagggccgcgtggagatacagcgagctggtgaatggggcaccatctgcgatgatgacttcacgctgcaggctgcccacatcctctgccgggagctgggcttcacagaggccacaggctggacccacagtgccaaatatggccctggaacaggccgcatctggctggacaacttgagctgcagtgggaccgagcagagtgtgactgaatgtgcctcccggggctgggggaacagtgactgtacgcacgatgaggatgctggggtcatctgcaaagaccagcgcctccctggcttctcggactccaatgtcattgaggtagagcatcacctgcaagtggaggaggtgcgaattcgacccgccgttgggtggggcagacgacccctgcccgtgacggaggggctggtggaagtcaggcttcctgacggctggtcgcaagtgtgcgacaaaggctggagcgcccacaacagccacgtggtctgcgggatgctgggcttccccagcgaaaagagggtcaacgcggccttctacaggctgctagcccaacggcagcaacactcctttggtctgcatggggtggcgtgcgtgggcacggaggcccacctctccctctgttccctggagttctatcgtgccaatgacaccgccaggtgccctggggggggccctgcagtggtgagctgtgtgccaggccctgtctacgcggcatccagtggccagaagaagcaacaacagtcgaagcctcagggggaggcccgtgtccgtctaaagggcggcgcccaccctggagagggccgggtagaagtcctgaaggccagcacatggggcacagtctgtgaccgcaagtgggacctgcatgcagccagcgtggtgtgtcgggagctgggcttcgggagtgctcgagaagctctgagtggcgctcgcatggggcagggcatgggtgctatccacctgagtgaagttcgctgctctggacaggagctctccctctggaagtgcccccacaagaacatcacagctgaggattgttcacatagccaggatgccggggtccggtgcaacctaccttacactggggcagagaccaggatccgactcagtgggggccgcagccaacatgaggggcgagtcgaggtgcaaatagggggacctgggccccttcgctggggcctcatctgtggggatgactgggggaccctggaggccatggtggcctgtaggcaactgggtctgggctacgccaaccacggcctgcaggagacctggtactgggactctgggaatataacagaggtggtgatgagtggagtgcgctgcacagggactgagctgtccctggatcagtgtgcccatcatggcacccacatcacctgcaagaggacagggacccgcttcactgctggagtcatctgttctgagactgcatcagatctgttgctgcactcagcactggtgcaggagaccgcctacatcgaagaccggcccctgcatatgttgtactgtgctgcggaagagaactgcctggccagctcagcccgctcagccaactggccctatggtcaccggcgtctgctccgattctcctcccagatccacaacctgggacgagctgacttcaggcccaaggctgggcgccactcctgggtgtggcacgagtgccatgggcattaccacagcatggacatcttcactcactatgatatcctcaccccaaatggcaccaaggtggctgagggccacaaagctagtttctgtctcgaagacactgagtgtcaggaggatgtctccaagcggtatgagtgtgccaactttggagagcaaggcatcactgtgggttgctgggatctctaccggcatgacattgactgtcagtggattgacatcacggatgtgaagccaggaaactacattctccaggttgtcatcaacccaaactttgaagtagcagagagtgactttaccaacaatgcaatgaaatgtaactgcaaatatgatggacatagaatctgggtgcacaactgccacattggtgatgccttcagtgaagaggccaacaggaggtttgaacgctaccctggccagaccagcaaccagattatctaa

SEQ ID NO: 19 - DNA, 355 bp, Homo sapiens
atggagagcgcagcacagttaggcccccaggtcccagtggctctcagttggatgagggaccaagggcagggccattgcatcacgaccctgtgctgttttccagagaggtatgctggacgggaccataacagctgcaaactctcccagagggggttcctaaacttcatgaacactgtactggttgccttcacaaagaaccagaagggctctggtgcccttgactgcatgatgaagaaactggacttcaactgtgatgggcaggattttcaggactttctcagtcttactgatggtgtagctgtggcttgccctgactccttcatcccggctggccatgccccatgagagaatctga

SEQ ID NO: 20 - DNA, 321 bp, Homo sapiens
atggcaaaaatctccggctgcacagagattgcatggtggtgcatcacgaccctgtgctgttttccagagaggtatgctggacgggaccataacagctgcaaactctcccagagggggttcctaaacttcatgaacactgtactggttgccttcacaaagaaccagaagggctctggtgcccttgactgcatgatgaagaaactggacttcaactgtgatgggcagctagattttcaggactttctcagtcttactgatggtgtagctgtggcttgccctgactccttcatcccggctggccatgcccatgagagaatctga

SEQ ID NO: 21 - DNA, 1932 bp, Homo sapiens
atggcccttgccggcccctgcccctcctccactgcttcccttctcccctccacccaagccttgcccacaattaactcatttcttaagatcgcttccaaacctaagtcaacgctggacagggctgtaggaaaagcttcctcaatactggccctgaagagccgagccagcgccaagaggagtgtgctgctccccatcctggcactgtgggcggggagctgctcaggaggggccccaccaacccccatgggcttggctaccctgcagctgctgcccagcccaccaggggcccccgacggtcagctgcagcccatccctggcatcggccacccagacaagcctgaggctgggaagctggaccagttgcgggatcagcccaccccgaagcagggagctcaaggaacccccacccagtccccctccactggctggaaagcgcttcccaggccagggctggccctgaggaaggagtcacccccagtgaccttggagcaggagcagggtcacaacaagggcctggtcgctgagtgggctcagccccaggccacagctgccatgagggctggggcagggaagcccgaggccttgaagctgaggccctggcaggccggcagggaccctcaagctcaagagggggcagcagtcaccgaggaggaccagggccagaggacaggaggccgggaagacaagggaaggggcctgaaacccaggaggccccccaaagggacctcccatcaacctgggctgaggatccggcgcccacagaaggaccgcagccgaggccagggtggcggcggcagcacctccaagaccccaggccatgggtggaaaagaccaggaagcacacatgggcacaggcacaggcacgcagacctgggcaccacccagcaggccatgccctctctgccggcctcgtgcctcctggcccaggcagtcatcgcctgtggcaatgtcaagatgaagcatgtccctgccctgacccaccctggtctgaccacactctacctggcagagaatgaaattgccaagatcccagcccacacgttcctggggctgcccaacctggagtggctggatctcagcaagaacaagctggatccccgaggcctgcacccccatgccttcaagaatctgatgcggctgaagcggctgaacctggttgggaactcgctgaccacagtcccggccctacctgcctccctgcaggagctcaaactcaacgacaacctcctgcagggcttgcaaggcagcagcttccgtgggctcagccagctgttgacgctggaggagctgcacctgggcaccaacctcatcgaggaggtggcggagggcgcactgagccacatccacagcctcagcgtgctggtgctcagccacaactggcttcaggagcactggctggcaccccgagcctggattcatctcccgaagctggagacccttgacctgtcctacaaccggctggtgcacgtgccccgcttcctgccgcggggcctgaggcgcctgacgctgcaccacgaccacatcgagcgcatccctggctacgcgttcgcgcacatgaagccaggcctagagttcctgcacctgtcccacaacaggctgcaggctgacggcatccacagcgtgtccttcctgggcctgcgcgcctcgctggcggagctgctcctggatcataaccaggtgcaggccatcccacgcggcctcctgggcctcaagggactgcaggtgctgggcctgagccacaacaggatcagacaagtgcccttgaattccatctgtgacatgcgcgtggctcaggactccaaccttacctccacacacctggagaacaacctcattgaccggcgccgcatcccgcccactgccttctcctgcacccgagcctatcacagcgtggtcctccagccccagcggcggggggaggagggctcctag

SEQ ID NO: 22 - DNA, 1962 bp, Homo sapiens
atggccgggtgccctgggactggacagagtgggcagcaggagtaccactccccaggggcccacccagccaagaggagtgtgctgctccccatcctggcactgtgggcggggagctgctcaggaggggccccaccaacccccatgggcttggctaccctgcagctgctgcccagcccaccaggggcccccgacggtcagctgcagcccatccctggcatcggccacccagacaagcctgaggctgggaagctggaccagttgcgggatcagcccaccccgaagcagggagctcaaggaacccccacccagtccccctccactggctggaaagcgcttcccaggccagggctggccctgaggaaggagtcacccccagtgaccttggagcaggagcagggtcacaacaagggcctggtcgctgagtgggctcagccccaggccacagctgccatgagggctggggcagggaagcccgaggccttgaagctgaggccctggcaggccggcagggaccctcaagctcaagagggggcagcagtcaccgaggaggaccagggccagaggacaggaggccgggaagacaagggaaggggcctgaaacccaggaggccccccaaagggacctcccatcaacctgggctgaggatccggcgcccacagaaggaccgcagccgaggccagggtggcggcggcagcacctccaagaccccaggccatgggtggaaaagaccaggaagcacacatgggcacaggcacaggcacgcagacctgggcaccacccagcaggccatgccctctctgccggcctcgtgcctcctggcccaggcagtcatcgcctgtggcaatgtcaagatgaagcatgtccctgccctgacccaccctggtctgaccacactctacctggcagagaatgaaattgccaagatcccagcccacacgttcctggggctgcccaacctggagtggctggatctcagcaagaacaagctggatccccgaggcctgcacccccatgccttcaagaatctgatgcggctgaagcggctgaacctggttgggaactcgctgaccacagtcccggccctacctgcctccctgcaggagctcaaactcaacgacaacctcctgcagggcttgcaaggcagcagcttccgtgggctcagccagctgttgacgctggaggtggaagggaaccagctgcgtgacagggacatctcccccctggccttccagcccctctgcagcctgctctatctgaggctggaccggaaccggctgcgggccatcccacgcggcctgccgtcctccctgcaggaactgcacctgggcaccaacctcatcgaggaggtggcggagggcgcactgagccacatccacagcctcagcgtgctggtgctcagccacaactggcttcaggagcactggctggcaccccgagcctggattcatctcccgaagctggagacccttgacctgtcctacaaccggctggtgcacgtgccccgcttcctgccgcggggcctgaggcgcctgacgctgcaccacgaccacatcgagcgcatccctggctacgcgttcgcgcacatgaagccaggcctagagttcctgcacctgtcccacaacaggctgcaggctgacggcatccacagcgtgtccttcctgggcctgcgcgcctcgctggcggagctgctcctggatcataaccaggtgcaggccatcccacgcggcctcctgggcctcaagggactgcaggtgctgggcctgagccacaacaggatcagacaagtgcccttgaattccatctgtgacatgcgcgtggctcaggactccaaccttacctccacacacctggagaacaacctcattgaccggcgccgcatcccgcccactgccttctcctgcacccgagcctatcacagcgtggtcctccagccccagcggcggggggaggagggctcctag

SEQ ID NO: 23 - DNA, 918 bp, Homo sapiens
atgggcgcgcgcggggcgctgctgctggcgctgctgctggctcgggctggactcgggaagccggagtcgcaggaggaggagctgttgtcagaggcctgcggccaccgggaaattcacgcgctggtggcgggcggagtggagtccgcgcgcgggcgctggccatggcaggccagcctgcgcctgaggagacgccaccgatgtggagggagcctgctcagccgccgctgggtgctctcggctgcgcactgcttccaaaagcactactatccctccgagtggacggtccagctgggcgagctgacttccaggccaactccttggaacctgcgggcctacagcagtcgttacaaagtgcaggacatcattgtgaaccctgacgcacttggggttttacgcaatgacattgccctgctgagactggcctcttctgtcacctacaatgcgtacatccagcccatttgcatcgagtcttccaccttcaacttcgtgcaccggccggactgctgggtgaccggctgggggttaatcagccccagtggcacacctctgccacctccttacaacctccgggaagcacaggtcaccatcttaaacaacaccaggtgtaattacctgtttgaacagccctctagccgtagtatgatctgggattccatgttttgtgctggtgctgaggatggcagtgtagacacctgcaaaggtgactcaggtggacccttggtctgtgacaaggatggactgtggtatcaggttggaatcgtgagctggggaatggactgcggtcaacccaatcggcctggtgtctacaccaacatcagtgtgtacttccactggatccggagggtgatgtcccacagtacacccaggccaaacccctcccagctgttgctgctccttgccctgctgtgggctccctga

SEQ ID NO: 24 - DNA, 1164 bp, Homo sapiens
atgagggtcacctggaaccacgggccgccatgtccctcccccgacagcttgacaataacctgtaattatggaaacggaggctgccagcacagctgtgaggacacagacacaggccccacgtgtggttgccaccagaagtacgccctccactcagacggtcgcacgtgcatcgagaaggatgaggctgcaattgagcgctctcagttcaatgccacgtcagtagctgatgtggacaagcgggtgaaacggcggctactcatggcaccccctgactgggggcagaagctaggtcttttccagcttggtgccccccctcagggcacagcacagggccttgcccagagcgggagcatggagtccctgctcattaatctagtcattgagcacaactcattagacacctccgccgtgctggtcaccttgacgctgccctgcccagatagcgtgtggtcagtgggagaggcctctgcacacacagacagcgctgccctgtggggcagaagcccaggggtgagcgctctccccaccagctggaggaggaagccagggcaccagcgggtgcagacctcacgtcccaggcgcctgagccgccctccacaagtgtgtttcagggtgggggagattcctcatgaggccataatgtcagcccctgagacgtgcgcagtcaataacggaggctgcgaccggacatgcaaggacacagccactggcgtgcgatgcagctgccccgttggattcacactgcagccggacgggaagacatgcaaagacatcaacgagtgcctggtcaacaacggaggctgcgaccacttctgccgcaacaccgtgggcagcttcgagtgcggctgccggaagggctacaagctgctcaccgacgagcgcacctgccaggacatcgacgagtgctccttcgagcggacctgtgaccacatctgcatcaactccccgggcagcttccagtgcctgtgtcaccgcggctacatcctctacgggacaacccactgcggagatgtggacgagtgcagcatgagcaacgggagctgtgaccagggctgcgtcaacaccaagggcagctacgagtgcgtctgtcccccggggaggcggctccactggaacgggaaggattgcgtgggcagaggttctctgctgttggggtatggctga

SEQ ID NO: 25 - DNA, 2895 bp, Homo sapiens
atgggcgcggcggccgtgcgctggcacttgtgcgtgctgctggccctgggcacacgcgggcggctggccgggggcagcgggctcccaggttcagtcgacgtggatgagtgctcagagggcacagatgactgccacatcgatgccatctgtcagaacacgcccaagtcctacaaatgcctctgcaagccaggctacaagggggaaggcaagcagtgtgaagacattgacgagtgtgagaatgactactacaatgggggctgtgtccacgagtgcatcaacatcccggggaactacaggtgtacctgctttgatggcttcatgctggcacacgatggacacaactgcctggatgtggacgagtgtcaggacaataatggtggctgccagcagatctgcgtcaatgccatgggcagctacgagtgtcagtgccacagtggcttcttccttagtgacaaccagcatacctgcatccaccgctccaatgagggtatgaactgcatgaacaaagaccatggctgtgcccacatctgccgggagacgcccaaaggtggggtggcctgcgactgcaggcccggctttgaccttgcccaaaaccagaaggactgcacactaacctgtaattatggaaacggaggctgccagcacagctgtgaggacacagacacaggccccacgtgtggttgccaccagaagtacgccctccactcagacggtcgcacgtgcatcgagacgtgcgcagtcaataacggaggctgcgaccggacatgcaaggacacagccactggcgtgcgatgcagctgccccgttggattcacactgcagccggacgggaagacatgcaaagacatcaacgagtgcctggtcaacaacggaggctgcgaccacttctgccgcaacaccgtgggcagcttcgagtgcggctgccggaagggctacaagctgctcaccgacgagcgcacctgccaggacatcgacgagtgctccttcgagcggacctgtgaccacatctgcatcaactccccgggcagcttccagtgcctgtgtcaccgcggctacatcctctacgggacaacccactgcggagatgtggacgagtgcagcatgagcaacgggagctgtgaccagggctgcgtcaacaccaagggcagctacgagtgcgtctgtcccccggggaggcggctccactggaacgggaaggattgcgtggagacaggcaagtgtctttctcgcgccaagacctccccccgggcccagctgtcctgcagcaaggcaggcggtgtggagagctgcttcctttcctgcccggctcacacactcttcgtgccagactcggaaaatagctacgtcctgagctgcggagttccagggccgcagggcaaggcgctgcagaaacgcaacggcaccagctctggcctcgggcccagctgctcagatgcccccaccacccccatcaaacagaaggcccgcttcaagatccgagatgccaagtgccacctccggccccacagccaggcacgagcaaaggagaccgccaggcagccgctgctggaccactgccatgtgactttcgtgaccctcaagtgtgactcctccaagaagaggcgccgtggccgcaagtccccatccaaggaggtgtcccacatcacagcagagtttgagatcgagacaaagatggaagaggcctcagacacatgcgaagcggactgcttgcggaagcgagcagaacagagcctgcaggccgccatcaagaccctgcgcaagtccatcggccggcagcagttctatgtccaggtctcaggcactgagtacgaggtagcccagaggccagccaaggcgctggaggggcagggggcatgtggcgcaggccaggtgctacaggacagcaaatgcgttgcctgtgggcctggcacccacttcggtggtgagctcggccagtgtgtgtcatgtatgccaggaacataccaggacatggaaggccagctcagttgcacaccgtgccccagcagcgacgggcttggtctgcctggtgcccgcaacgtgtcggaatgtggaggccagtgttctccaggcttcttctcggccgatggcttcaagccctgccaggcctgccccgtgggcacgtaccagcctgagcccgggcgcaccggctgcttcccctgtggagggggtttgctcaccaaacacgaaggcaccacctccttccaggactgcgaggctaaagtgcactgctcccccggccaccactacaacaccaccacccaccgctgcatccgctgccccgtcggcacctaccagcccgagtttggccagaaccactgcatcacctgtccgggcaacaccagcacagacttcgatggctccaccaacgtcacacactgcaaaaaccagcactgcggcggcgagcttggtgactacaccggctacatcgagtcccccaactaccctggcgactacccagccaacgctgaatgcgtctggcacatcgcgcctcccccaaagcgcaggatcctcatcgtggtccctgagatcttcctgcccatcgaggatgagtgcggcgatgttctggtcatgaggaagagtgcctctcccacgtccatcaccacctatgagacctgccagacctacgagaggcccatcgccttcacctcccgctcccgcaagctctggatccagttcaaatccaatgaaggcaacagcggcaaaggcttccaagtgccctatgtcacctacgatggtaagatccactgtcttcacggcccactgtgcacggctcaggcggggccctggagacacagagatgagtcgcacgtccccgccctcagggagctgcgacctggcaggtacagacctggaagcagaacgaacactgtcaggggccagagccagacaggctga

SEQ ID NO: 26 - DNA, 640 bp, Homo sapiens
aatggttttaccctcatattcaaaatcagagggagggtcattattggatatctactgtttactcacgtattggatggaggtggtgcccaccctcttggcagagacaaagattccagccactgatgtcgctgatgccagcctgaatgaatgttccagtaccgaaaggaaacaagacgtagtgttgctgttcgtgaccttgtcccacacacagccacctctgtttcacctgccttatgtccagaaacccttaatctctaatgtggagcagctgatcctggggatcccgggccagaatcgccgggagataggccatggccaggatatctttccagcagagaagctctgccatctgcaggatcgcaaggtgaaccttcacagagctgcctggggcgagtgtattgttgcacccaagactctcagcttctcttactgtcaggggacctgcccggccctcaacagtgagctccgtcattccagctttgagtgctataagagggcagtacctacctgtccctggctcttccagacctgccgtcccaccatggtcagactcttctccctgatggtccaggatgacgaacacaagatgagtgtgcactatgtgaacacttccttggtggagaagtgtggctgctcttga

SEQ ID NO: 27 - DNA, 568 bp, Homo sapiens
batggaggtggtgcccaccctcttggcagagacaaagattccagccactgatgtcgctgatgccagcctgaatgaatgttccagtaccgaaaggaaacaagacgtagtgttgctgttcgtgaccttgtcccacacacagccacctctgtttcacctgccttatgtccagaaacccttaatctctaatgtggagcagctgatcctggggatcccgggccagaatcgccgggagataggccatggccaggatatctttccagcagagaagctctgccatctgcaggatcgcaaggtgaaccttcacagagctgcctggggcgagtgtattgttgcacccaagactctcagcttctcttactgtcaggggacctgcccggccctcaacagtgagctccgtcattccagctttgagtgctataagagggcagtacctacctgtccctggctcttccagacctgccgtcccaccatggtcagactcttctccctgatggtccaggatgacgaacacaagatgagtgtgcactatgtgaacacttccttggtggagaagtgtggctgctcttga

SEQ ID NO: 28 - DNA, 2223 bp, Homo sapiens
atgggtgactcaggagcagaggctgtgggaggtggggggacatacactgatggccccgtgctcctcctctatgcaggggagctgctgttgccccaggagacgactgtggagctgagctgtggagtggggccactgcaagtgatcctgggcccagagcaggctgcagtgctaaactgtagcctgggggctgctgccgctggaccccccaccagggtgacctggagcaaggatggggacaccctgctggagcacgaccacttacacctgctgcccaatggttccctgtggctgtcccagccactagcacccaatggcagtgacgagtcagtccctgaggctgtgggggtcattgaaggcaactattcgtgcctagcccacggcccccctggagtgctggccagccagactgctgtcgtcaagcttgccacactcgcagacttctctctgcacccggagtctcagacggtggaggagaacgggacagctcgctttgagtgccacattgaagggctgccagctcccatcattacttgggagaaggaccaggtgacattgcctgaggagcctcggctcatcgtgcttcccaacggcgtccttcagatcctggatgttcaggagagtgatgcaggcccctaccgctgcgtggccaccaactcagctcgccagcacttcagccaggaggccctactcagtgtggcccacagagggtccctggcgtccaccagggggcaggacgtggtcattgtggcagccccagagaacaccacagtggtgtctggccagagtgtggtgatggaatgtgtggcctcagctgaccccaccccttttgtgtcctgggtccgacaagacgggaagcccatctccacagatgtcatcgtcctgggccgcaccaacctactaattgccaacgcgcagccctggcactccggcgtctatgtctgccgcgccaacaagccccgcacgcgcgacttcgccactgcagccgctgagctccgtgtgctggcggctcccgccatcactcaggcgcccgaggcgctgtcgcggacgcgggcgagcacagcgcgcttcgtgtgccgcgcgtcgggggagccgcggccagcgctgcgctggctgcacaacggggcgccgctgcggcccaacgggcgcgtcaaggtccagggcggcggtggcagcctggtcatcacacagatcggcctgcaggacgccggctactaccagtgcgtggctgagaacagcgcgggaatggcgtgcgctgccgcgtcgctggccgtggtggtgcgcgaggggctgcccagcgcccccacgcgggtcactgctacgccactgagcagctccgctgtgttggtggcctgggagcggcccgagatgcacagcgagcagatcatcggcttctctctccactaccagaaggcacggggcatggacaatgtggaataccagtttgcagtgaacaacgacaccacagaactacaggttcgggacctggaacccaacacagattatgagttctacgtggtggcctactcccagctgggagccagccgcacctccaccccagcactggtgcacacactggatgatggtagggcctctgaactcgcagtgggcagcttgggcctgagcaatgggcaggtggtgaagtacaagatagaatacggtttgggaaaggaagatcagattttctctactgaggtgcgaggaaatgagacacagcttatgctgaactcgcttcagccaaacaaggtgtatcgagtacggatttcggctggtacagcagccggcttcggggccccctcccagtggatgcatcacaggacgcccagtatgcacaaccagagccatgtcccttttgcccctgcagagttgaaggtgcaggcaaagatggagtccctggtcgtgtcatggcagccaccccctcaccccacccagatctctggctacaaactatattggcgggaggtgggggctgaggaggaggccaatggcgatcgcctgccagggggccgtggagaccaggcttgggatgtggggcctgtccggctcaagaagaaagtgaagcagtatgagctgacccagctagtccctggccggctgtacgaggtgaagctcgtggctttcaacaaacatgaggatggctatgcagcagtgtggaagggcaagacggagaaggcgccggcaccaggtgagggcggtgggggaagaaggcggggagggctcaggtga

SEQ ID NO: 29 - DNA, 3753 bp, Homo sapiens
atggcgcggggggacgccggccgcggccgcgggctcctcgcgttgaccttctgcctgttggccgcgcgcggggagctgctgttgccccaggagacgactgtggagctgagctgtggagtggggccactgcaagtgatcctgggcccagagcaggctgcagtgctaaactgtagcctgggggctgctgccgctggaccccccaccagggtgacctggagcaaggatggggacaccctgctggagcacgaccacttacacctgctgcccaatggttccctgtggctgtcccagccactagcacccaatggcagtgacgagtcagtccctgaggctgtgggggtcattgaaggcaactattcgtgcctagcccacggccccctcggagtgctggccagccagactgctgtcgtcaagcttgccacactcgcagacttctctctgcacccggagtctcagacggtggaggagaacgggacagctcgctttgagtgccacattgaagggctgccagctcccatcattacttgggagaaggaccaggtgacattgcctgaggagcctcggctcatcgtgcttcccaacggcgtccttcagatcctggatgttcaggagagtgatgcaggcccctaccgctgcgtggccaccaactcagctcgccagcacttcagccaggaggccctactcagtgtggcccacagagggtccctggcgtccaccagggggcaggacgtggtcattgtggcagccccagagaacaccacagtggtgtctggccagagtgtggtgatggaatgtgtggcctcagctgaccccaccccttttgtgtcctgggtccgacaagacgggaagcccatctccacagatgtcatcgtcctgggccgcaccaacctactaattgccaacgcgcagccctggcactccggcgtctatgtctgccgcgccaacaagccccgcacgcgcgacttcgccactgcagccgctgagctccgtgtgctggcggctcccgccatcactcaggcgcccgaggcgctgtcgcggacgcgggcgagcacagcgcgcttcgtgtgccgcgcgtcgggggagccgcggccagcgctgcgctggctgcacaacggggcgccgctgcggcccaacgggcgcgtcaaggtccagggcggcggtggcagcctggtcatcacacagatcggcctgcaggacgccggctactaccagtgcgtggctgagaacagcgcgggaatggcgtgcgctgccgcgtcgctggccgtggtggtgcgcgaggggctgcccagcgcccccacgcgggtcactgctacgccactgagcagctccgctgtgttggtggcctgggagcggcccgagatgcacagcgagcagatcatcggcttctctctccactaccagaaggcacggggcatggacaatgtggaataccagtttgcagtgaacaacgacaccacagaactacaggttcgggacctggaacccaacacagattatgagttctacgtggtggcctactcccagctgggagccagccgcacctccaccccagcactggtgcacacactggatgatgtccccagtgcagcaccccagctctccctgtccagccccaacccttcggacatcagggtggcgtggctgcccctgccccccagcctgagcaatgggcaggtggtgaagtacaagatagaatacggtttgggaaaggaagatcagattttctctactgaggtgcgaggaaatgagacacagcttatgctgaactcgcttcagccaaacaaggtgtatcgagtacggatttcggctggtacagcagccggcttcggggccccctcccagtggatgcatcacaggacgcccagtatgcacaaccagagccatgtcccttttgcccctgcagagttgaaggtgcaggcaaagatggagtccctggtcgtgtcatggcagccaccccctcaccccacccagatctctggctacaaactatattggcgggaggtgggggctgaggaggaggccaatggcgatcgcctgccagggggccgtggagaccaggcttgggatgtggggcctgtccggctcaagaagaaagtgaagcagtatgagctgacccagctagtccctggccggctgtacgaggtgaagctcgtggctttcaacaaacatgaggatggctatgcagcagtgtggaagggcaagacggagaaggcgccggcaccagacatgcctatccagaggggaccacccctgcctccagcccacgtccatgcggaatcaaacagctccacatccatctggcttcggtggaaaaagccagatttcaccacagtcaagattgtcaactacactgtgcgcttcagcccctgggggctcaggaatgcctccctggtcacctattacaccagttctggagaagacatcctcattggcggcttgaagccattcaccaaatacgagtttgcagtgcagtctcacggcgtggacatggatgggcctttcggctctgtggtggagcgctccaccctgcctgaccggccctccacacccccatccgacctgcgactgagccccctgacaccgtccacggttcggctgcactggtgcccccccacagagcccaacggggagatcgtggagtatctgatcctgtacagcagcaaccacacgcagcctgagcaccagtggaccttgctcaccacgcagggaaacatcttcagtgctgaggtccatggcctggagagcgacactcggtacttcttcaagatgggggcgcgcacagaggtgggacctgggcctttctcccgcctgcaggatgtgatcacgctccaggagaagctgtcagactcgctggacatgcactcagtcacgggcatcatcgtgggtgtctgcctgggcctcctctgcctcctggcctgcatgtgtgctggcctgcgccgcagcccccacagggaatccctcccaggcctgtcctccaccgccacccccgggaatcccgcgctgtactccagagctcggcttggcccccccagccccccagctgcccatgaattggagtcccttgtgcacccccatccccaggactggtccccgccaccctcagacgtggaggacagggctgaagtgcacagccttatgggtggcggtgtttctgaaggccggagtcactccaaaagaaagatctcctgggctcaaccaagcgggctgagctgggctggttcctgggcaggctgtgagctgccccaggcaggcccccggccggctctgacccgggccctgctgccccctgctggaactgggcagacgctgttgctgcaggctctggtgtacgacgccataaagggcaatgggaggaagaagtcacccccagcctgcaggaaccaggtggaggctgaagtcattgtccactctgactttagtgcatctaacgggaaccctgacctccatctccaagacctggagcctgaggaccccctgcctccagaggctcctgatctcatctcgggtgttggggatccagggcagggggcagcctggctggacagggagttgggagggtgtgagctggcagcccccgggccagacagacttacctgcttgccagaggcagccagtgcttcctgctcctacccggacctccagccaggcgaggtgctagaggagacccctggagatagctgccagctcaaatccccctgccctctaggagccagcccaggcctgcccagatccccggtctcctcctctgcctag

SEQ ID NO: 30 - DNA, 1905 bp, Homo sapiens
atggcccagggtgtcctctggatcctactcggattgctactgtggtcagacccagggacagcctccctgcccctgctcatggactctgtcatccaggccctggctgagctggagcagaaagtgccagctgccaagaccagacacacagcttctgcgtggctgatgtcagctccaaactctggcccccacaatcgcctctaccacttcctgctgggggcatggagcctcaatgctacagagttggatccctgcccactaagcccagagctgttaggcctgaccaaggaggtggcccgacatgacgtacgagaagggaaggaatatggggtggtgctggcacctgatggctcgaccgtggctgtggagcctctgctggcggggctggaggcagggctgcaagggcgcagggtcataaatttgcccttggacagcatggctgccccttgggagactggagatacctttccagatgttgtggccattgctccagatgtaagagccacctcctccccaggactcagggatggctctccagatgtcaccactgcagatattggagccaacactccagatgctacaaaaggctgtccagatgtccaagcttccttgccagatgccaaagccaagtccccaccgaccatggtggacagcctcctggcagtcaccctggctggaaacctgggcctgaccttcctccgaggttcccagacccagagccatccagacctgggaactgagggctgctgggaccagctctctgcccctcggacctttacgcttttggaccccaaggcatctctgttaaccatggccttcctcaatggcgccctggatggggtcatccttggagactacctgagccggactcctgagccccggccatccctcagccacttgctgagccagtactatggggctggggtggccagagacccagggttccgcagcaacttccgacggcagaacggtgctgctctgacttcagcctccatcctggcccagcaggtgtggggaacccttgtccttctacagaggctggagccagtacacctccagcttcagtgcatgagccaagaacagctggcccaggtggctgccaatgctaccaaggaattcactgaggccttcctgggatgcccggccatccacccccgctgccgctggggagcggcgccttatcggggccgcccgaagctgctgcagctgccgctgggattcttgtacgtgcatcacacctacgtgcctgcaccaccctgcacggacttcacgcgctgcgcagccaacatgcgctccatgcagcgctaccaccaggacacgcaaggctggggagacatcggctacagtttcgtggtgggctcggacggctacgtgtacgagggacgcggctggcactgggtgggcgcccacacgctcggccacaactcccggggcttcggcgtggccatagtgggcaactacaccgcggcgctgcccaccgaggccgctctgcgcacggtgcgcgacacgctcccgagttgtgcggtgcgcgccggcctcctgcggccagactacgcgctgctgggccaccgccagctggtgcgcaccgactgccccggcgacgcgctcttcgacctgctgcgcacctggccgcacttcaccgcggtgagtcttcgcagcctgcactacacggcccgccgcccctccgtctacacaagctccacgaggcccctgccccctgcctgtaacagctgtgcccgcacagcctcagccaggcccccaacttcccggcggcacgtctattcaggaaacctaggcccagcctttgcgggtcactctgcgggcaacatccctgatcctgtgacttctgcctatgcagcctcagctcagccccagacccagccagcctgtcctttccccagctcctaa

SEQ ID NO: 31 - DNA, 1731 bp, Homo sapiens
atggcccagggtgtcctctggatcctactcggattgctactgtggtcagacccagggacagcctccctgcccctgctcatggactctgtcatccaggccctggctgagctggagcagaaagtgccagctgccaagaccagacacacagcttctgcgtggctgatgtcagctccaaactctggcccccacaatcgcctctaccacttcctgctgggggcatggagcctcaatgctacagagttggatccctgcccactaagcccagagctgttaggcctgaccaaggaggtggcccgacatgacgtacgagaagggaaggaatatggggtggtgctggcacctgatggctcgaccgtggctgtggagcctctgctggcggggctggaggcagggctgcaagggcgcagggtcataaatttgcccttggacagcatggctgccccttgggagactggagatacctttccagatgttgtggccattgctccagatgtaagagccacctcctccccaggactcagggatggctctccagatgtcaccactgcagatattggagccaacactccagatgctacaaaaggctgtccagatgtccaagcttccttgccagatgccaaagccaagtccccaccgaccatggtggacagcctcctggcagtcaccctggctggaaacctgggcctgaccttcctccgaggttcccagacccagagccatccagacctgggaactgagggctgctgggaccagctctctgcccctcggacctttacgcttttggaccccaaggcatctctgttaaccatggccttcctcaatggcgccctggatggggtcatccttggagactacctgagccggactcctgagccccggccatccctcagccacttgctgagccagtactatggggctggggtggccagagacccagggttccgcagcaacttccgacggcagaacggtgctgctctgacttcagcctccatcctggcccagcaggtgtggggaacccttgtccttctacagaggctggagccagtacacctccagcttcagtgcatgagccaagaacagctggcccaggtggctgccaatgctaccaaggaattcactgaggccttcctgggatgcccggccatccacccccgctgccgctggggagcggcgccttatcggggccgcccgaagctgctgcagctgccgctgggattcttgtacgtgcatcacacctacgtgcctgcaccaccctgcacggacttcacgcgctgcgcagccaacatgcgctccatgcagcgctaccaccaggacacgcaaggctggggagacatcggctacagtttcgtggtgggctcggacggctacgtgtacgagggacgcggctggcactgggtgggcgcccacacgctcggccacaactcccggggcttcggcgtggccatagtgggcaactacaccgcggcgctgcccaccgaggccgctctgcgcacggtgcgcgacacgctcccgagttgtgcggtgcgcgccggcctcctgcggccagactacgcgctgctgggccaccgccagctggtgcgcaccgactgccccggcgacgcgctcttcgacctgctgcgcacctggccgcacttcaccgcgactgttaagccaagacctgccaggagtgtctctaagagatccaggagggagccacccccaaggaccctgccagccacagacctccaataa

SEQ ID NO: 32 - DNA, 2205 bp, Homo sapiens
atgtgggggctcctgctcgccctggccgccttcgcgccggccgtcggcccggctctgggggcgcccaggaactcggtgctgggcctcgcgcagcccgggaccaccaaggtcccaggctcgaccccggccctgcatagcagcccggcacagccgccggcggagacagctaacgggacctcagaacagcatgtccggattcgagtcatcaagaagaaaaaggtcattatgaagaagcggaagaagctaactctaactcgccccaccccactggtgactgccgggccccttgtgacccccactccagcagggaccctcgaccccgctgagaaacaagaaacaggctgtcctcctttgggtctggagtccctgcgagtttcagatagccggcttgaggcatccagcagccagtcctttggtcttggaccacaccgaggacggctcaacattcagtcaggcctggaggacggcgatctatatgatggagcctggtgtgctgaggagcaggacgccgatccatggtttcaggtggacgctgggcaccccacccgcttctcgggtgttatcacacagggcaggaactctgtctggaggtatgactgggtcacatcatacaaggtccagttcagcaatgacagtcggacctggtggggaagtaggaaccacagcagtgggatggacgcagtatttcctgccaattcagacccagaaactccagtgctgaacctcctgccggagccccaggtggcccgcttcattcgcctgctgccccagacctggctccagggaggcgcgccttgcctccgggcagagatcctggcctgcccagtctcagaccccaatgacctattccttgaggcccctgcgtcgggatcctctgaccctctagactttcagcatcacaattacaaggccatgaggaagctgatgaagcaggtacaagagcaatgccccaacatcacccgcatctacagcattgggaagagctaccagggcctgaagctgtatgtgatggaaatgtcggacaagcctggggagcatgagctgggggagcctgaggtgcgctacgtggctggcatgcatgggaacgaggccctggggcgggagttgcttctgctcctgatgcagttcctgtgccatgagttcctgcgagggaacccacgggtgacccggctgctctctgagatgcgcattcacctgctgccctccatgaaccctgatggctatgagatcgcctaccaccggggttcagagctggtgggctgggccgagggccgctggaacaaccagagcatcgatcttaaccataattttgctgacctcaacacaccactgtgggaagcacaggacgatgggaaggtgccccacatcgtccccaaccatcacctgccattgcccacttactacaccctgcccaatgccaccgtggctcctgaaacgcgggcagtaatcaagtggatgaagcggatcccctttgtgctaagtgccaacctccacgggggtgagctcgtggtgtcctacccattcgacatgactcgcaccccgtgggctgcccgcgagctcacgcccacaccagatgatgctgtgtttcgctggctcagcactgtctatgctggcagtaatctggccatgcaggacaccagccgccgaccctgccacagccaggacttctccgtgcacggcaacatcatcaacggggctgactggcacacggtccccgggagcatgaatgacttcagctacctacacaccaactgctttgaggtcactgtggagctgtcctgtgacaagttccctcacgagaatgaattgccccaggagtgggagaacaacaaagacgccctcctcacctacctggagcaggtgcgcatgggcattgcaggagtggtgagggacaaggacacggagcttgggattgctgacgctgtcattgccgtggatgggattaaccatgacgtgaccacggcgtggggcggggattattggcgtctgctgaccccaggggactacatggtgactgccagtgccgagggctaccattcagtgacacggaactgtcgggtcacctttgaagagggccccttcccctgcaatttcgtgctcaccaagactcccaaacagaggctgcgcgagctgctggcagctggggccaaggtgcccccggaccttcgcaggcgcctggagcggctaaggggacagaaggattga

SEQ ID NO: 33 - DNA, 1077 bp, Homo sapiens
atgcctgaggatgtacgagaaaaaaaggaaaatcttctactcaattctgagagatctactaggctcttaacaaagaccagtcattcacaaggaggggatcaagctttaagtaagtccacagggtcaccaacagagaagttgattgaaaaacgtcaaggagctaagactgtttttaacaagttcagcaacatgaattggccagtggacattcaccctttaaacaaaagtttagtcaaagataataaatggaagaaaactgaggagacccaagagaaacgaaggtctttccttcaggagttttgcaagaaatacggtggggtgagtcatcatcagtcacatctttttcatacagtatccagaatctatgtagaagataaacacaaaatcttatattgtgaggtacctaaggctggctgttccaattggaaaagaattctgatggtactaaatggattggcttcctctgcatacaacatctcccacaatgctgtccactacgggaagcatttgaagaagctagatagctttgacctaaaagggatatatacccgcttaaatacttacaccaaagctgtgtttgttcgtgatcccatggaaagattagtatcagcctttagggacaaatttgaacaccccaatagttattaccatccagtattcggaaaggcaattatcaagaaatatcgaccaaatgcctgtgaagaagcattaattaatggatctggagtcaagttcaaagagtttatccactacttgctggattcccaccgtccagtaggaatggacattcactgggaaaaggtcagcaaactctgctatccgtgtttgatcaactatgattttgtagggaaatttgagactttggaagaagatgccaattactttttacagatgatcggtgctccaaaggagctgaaatttcccaactttaaggataggcactcttccgatgaaagaaccaatgctcaagtcgtgagacagtatttaaaggatctgactagaactgagagacaattaatctatgacttttattacttggactatttaatgtttaattatacaactccatttttgtag

SEQ ID NO: 34 - protein, 256 aa, Homo sapiens [sequence omitted]

SEQ ID NO: 35 - protein, 897 aa, Homo sapiens
MGSGRVPGLCLLVLLVHARAAQYSKAAQDVDECVEGTDNCHIDAICQNTPRSYKCICKSGYTGDGKHCKDVDECEREDNAGCVHDCVNIPGNYRCTCYDGFHLAHDGHNCLDVDECAEGNGGCQQSCVNMMGSYECHCREGFFLSDNQHTCIQRPEEGMNCMNKNHGCAHICRETPKGGIACECRPGFELTKNQRDCKCEIIGMAVTCNYGNGGCQHTCDDTEQGPRCGCHIKFVLHTDGKTCIETCAVNNGGCDSKCHDAATGVHCTCPVGFMLQPDRKTCKDIDECRLNNGGCDHICRNTVGSFECSCKKGYKLLINERNCQDIDECSFDRTCDHICVNTPGSFQCLCHRGYLLYGITHCGDVDECSINRGGCRFGCINTPGSYQCTCPAGQGRLHWNGKDCTEPLKCQGSPGASKAMLSCNRSGKKDTCALTCPSRARFLPESENGFTVSCGTPSPRAAPARAGHNGNSTNSNHCHEAAVLSIKQRASFKIKDAKCRLHLRNKGKTEEAGRITGPGGAPCSECQVTFIHLKCDSSRKGKGRRARTPPGKEVTRLTLELEAEQLFLLPDTHGHPPPASCGLPCLRQRMERRLKGSLKMLRKSINQDRFLLRLAGLDYELAHKPGLVAGERAEPMESCRPGQHRAGTKCVQCSPGHYYNTSIHRCIRCAMGSYQPDFRQNFCSRCPGNTSTDFDGSTSVAQCKNRQCGGELGEFTGYIESPNYPGNYPAGVECIWNINPPPKRKILIVVPEIFLPSEDECGDVLVMRKNSSPSSITTYETCQTYERPIAFTARSRKLWINFKTSEANSARGFQIPYVTYDEDYEQLVEDIVRDGRLYASENHQEILKDKKLIKAFFEVLAHPQNYFKYTEKHKEMLPKSFIKLLRSKVSSFLRPYK

SEQ ID NO: 36 - protein, 993 aa, Homo sapiens
MGSGRVPGLCLLVLLVHARAAQYSKAAQDVDECVEGTDNCHIDAICQNTPRSYKCICKSGYTGDGKHCKDVDECEREDNAGCVHDCVNIPGNYRCTCYDGFHLAHDGHNCLDVDECAEGNGGCQQSCVNMMGSYECHCREGFFLSDNQHTCIQRPEEGMNCMNKNHGCAHICRETPKGGIACECRPGFELTKNQRDCKLTCNYGNGGCQHTCDDTEQGPRCGCHIKFVLHTDGKTCIETCAVNNGGCDSKCHDAATGVHCTCPVGFMLQPDRKTCKDIDECRLNNGGCDHICRNTVGSFECSCKKGYKLLINERNCQDIDECSFDRTCDHICVNTPGSFQCLCHRGYLLYGITHCGDVDECSINRGGCRFGCINTPGSYQCTCPAGQGRLHWNGKDCTEPLKCQGSPGASKAMLSCNRSGKKDTCALTCPSRARFLPESENGFTVSCGTPSPRAAPARAGHNGNSTNSNHCHEAAVLSIKQRASFKIKDAKCRLHLRNKGKTEEAGRITGPGGAPCSECQVTFIHLKCDSSRKGKGRRARTPPGKEVTRLTLELEAEVRAEETTASCGLPCLRQRMERRLKGSLKMLRKSINQDRFLLRLAGLDYELAHKPGLVAGERAEPMESCRPGQHRAGTKCVSCPQGTYYHGQTEQCVPCPAGTFQEREGQLSCDLCPGSDAHGPLGATNVTTCAGQCPPGQHSVDGFKPCQPCPRGTYQPEAGRTLCFPCGGGLTTKHEGAISFQDCDTKVQCSPGHYYNTSIHRCIRCAMGSYQPDFRQNFCSRCPGNTSTDFDGSTSVAQCKNRQCGGELGEFTGYIESPNYPGNYPAGVECIWNINPPPKRKILIVVPEIFLPSEDECGDVLVMRKNSSPSSITTYETCQTYERPIAFTARSRKLWINFKTSEANSARGFQIPYVTYDEDYEQLVEDIVRDGRLYASENHQEILKDKKLIKAFFEVLAHPQNYFKYTEKHKEMLPKSFIKLLRSKVSSFLRPYK

SEQ ID NO: 37 - protein, 138 aa, Homo sapiens
MVRLCQALLLLVATVALASRRFQAWGSTKVVRTFQDIPQNYVYVQQALWFAMKEYNKASFSITSSALGKEYKLKVTDSLEYYIEVKIARTICKKISEDENCAFQEDPKMQKVVFCTFIVASKPWKFELTMLKKQCKDM

SEQ ID NO: 38 - protein, 241 aa, Homo sapiens
MKFILLWALLNLTVALAFNPDYTVSSTPPYLVYLKSDYLPCAGVLIHPLWVITAAHCNLPKLRVILGVTIPADSNEKHLQVIGYEKMIHHPHFSVTSIDHDIMLIKLKTEAELNDYVKLANLPYQTISENTMCSVSTWSYNVCDIYKEPDSLQTVNISVISKPQCRDAYKTYNITENMLCVGIVPGRRQPCKEVSAAPAICNGMLQGILSFADGCVLRADVGIYAKIFYYIPWIENVIQNN

SEQ ID NO: 39 - protein, 243 aa, Homo sapiens
MTEKSWNFLSMLLFPVALAFNPDYTVSSTPPYLVYLKSDYLPCAGVLIHPLWVITAAHCNLPKLRVILGVTIPADSNEKHLQVIGYEKMIHHPHFSVTSIDHDIMLIKLKTEAELNDYVKLANLPYQTISENTMCSVSTWSYNVCDIYKEPDSLQTVNISVISKPQCRDAYKTYNITENMLCVGIVPGRRQPCKEVSAAPAICNGMLQGILSFADGCVLRADVGIYAKIFYYIPWIENVIQNN

SEQ ID NO: 40 - protein, 483 aa, Homo sapiens
MYPGWPGQGMWASGQRLPDEAFESLTQLQHLCVAHNKLSVAPQFLPRSLRVADLAANQVMEIFPLTFGEKPALRSVYLHNNQLSNAGLPPDAFRGSEAIATLSLSNNQLSYLPPSLPPSLERLHLQNNLISKVPRGALSRQTQLRELYLQHNQLTDSGLDATTFSKLHSLEYLDLSHNQLTTVPAGLPRTLAILHLGRNRIRQVEAARLHGARGLRYLLLQHNQLGSSGLPAGALRPLRGLHTLHLYGNGLDRVPPALPRRLRALVLPHNHVAALGARDLVATPGLTELNLAYNRLASARVHHRAFRRLRALRSLDLAGNQLTRLPMGLPTGLRTLQLQRNQLRMLEPEPLAGLDQLRELSLAHNRLRVGDIGPGTWHELQALQMLDLSHNELSFVPPDLPEALEELHLEGNRIGHVGPEAFLSTPRLRALFLRANRLHMTSIAAEAFLGLPNLRVVDTAGNPEQVLIRLPPTTPRGPRAGGP

SEQ ID NO: 41 - protein, 605 aa, Homo sapiens
MAESGLAMEGMLQSPWRPCAQPGDTLTLPPPQWPSLLLLLLLPGPPPVAGLEDAAFPHLGESLQPLPRACPLRCSCPRVDTVDCDGLDLRVFPDNITRAAQHLSLQNNQLQELPYNELSRLSGLRTLNLHNNLISSEGLPDEAFESLTQLQHLCVAHNKLSVAPQFLPRSLRVADLAANQVMEIFPLTFGEKPALRSVYLHNNQLSNAGLPPDAFRGSEAIATLSLSNNQLSYLPPSLPPSLERLHLQNNLISKVPRGALSRQTQLRELYLQHNQLTDSGLDATTFSKLHSLEYLDLSHNQLTTVPAGLPRTLAILHLGRNRIRQVEAARLHGARGLRYLLLQHNQLGSSGLPAGALRPLRGLHTLHLYGNGLDRVPPALPRRLRALVLPHNHVAALGARDLVATPGLTELNLAYNRLASARVHHRAFRRLRALRSLDLAGNQLTRLPMGLPTGLRTLQLQRNQLRMLEPEPLAGLDQLRELSLAHNRLRVGDIGPGTWHELQALQMLDLSHNELSFVPPDLPEALEELHLEGNRIGHVGPEAFLSTPRLRALFLRANRLHMTSIAAEAFLGLPNLRVVDTAGNPEQVLIRLPPTTPRGPRAGGP

SEQ ID NO: 42 - protein, 1049 aa, Homo sapiens
MVTRELFFLFSPQFFSLNLRSHTRSTMTSPQLEWTLQTLLEQLNEDELKSFKSLLWAFPLEDVLQKTPWSEVEEADGKKLAEILVNTSSENWIRNATVNILEEMNLTELCKMAKAEMMEDGQVQEIDNPELGDAEEDSELAKPGEKEGWRNSMEKQSLVWKNTFWQGDIDNFHDDVTLRNQRFIPFLNPRTPRKLTPYTVVLHGPAGVGKTTLAKKCMLDWTDCNLSPTLRYAFYLSCKELSRMGPCSFAELISKDWPELQDDIPSILAQAQRILFVVDGLDELKVPPGALIQDICGDWEKKKPVPVLLGSLLKRKMLPRAALLVTTRPRALRDLQLLAQQPIYVRVEGFLEEDRRAYFLRHFGDEDQAMRAFELMRSNAALFQLGSAPAVCWIVCTTLKLQMEKGEDPVPTCLTRTGLFLRFLCSRFPQGAQLRGALRTLSLLAAQGLWAQMSVFHREDLERLGVQESDLRLFLDGDILRQDRVSKGCYSFIHLSFQQFLTALFYALEKEEGEDRDGHAWDIGDVQKLLSGEERLKNPDLIQVGHFLFGLANEKRAKELEATFGCRMSPDIKQELLQCKAHLHANKPLSVTDLKEVLGCLYESQEEELAKVVVAPFKEISIHLTNTSEVMHCSFSLKHCQDLQKLSLQVAKGVFLENYMDFELDIEFESSNSNLKFLEVKQSFLSDSSVRILCDHVTRSTCHLQKVEIKNVTPDTAYRDFCLAFIGKKTLTHLTLAGHIEWERTMMLMLCDLLRNHKCNLQYLRLGGHCATPEQWAEFFYVLKANQSLKHLRLSANVLLDEGAMLLYKTMTRPKHFLQMLSLENCRLTEASCKDLAAVLVVSKKLTHLCLAKNPIGDTGVKFLCEGLSYPDCKLQTLVLVSCSATTQQWADLSLALEVNQSLTCVNLSDNELLDEGAKLLYTTLRHPKCFLQRLSLENCHLTEANCKDLAAVLVVSRELTHLCLAKNPIGNTGVKFLCEGLRYPECKLQTLVLQQCSITKLGCRYLSEALQEACSLTNLDLSINQIARGLWILCQALENPNCNLKHLR

SEQ ID NO: 43 - protein, 1062 aa, Homo sapiens
MVSSAQMGFNLQALLEQLSQDELSKFKYLITTFSLAHELQKIPHKEVDKADGKQLVEILTTHCDSYWVEMASLQVFEKMHRMDLSERAKDEVREAALKSFNKRKPLSLGITRKERPPLDVDEMLERFKTEAQAFTETKGNVICLGKEVFKGKKPDKDNRCRYILKTKFREMWKSWPGDSKEVQVMAERYKMLIPFSNPRVLPGPFSYTVVLYGPAGLGKTTLAQKLMLDWAEDNLIHKFKYAFYLSCRELSRLGPCSFAELVFRDWPELQDDIPHILAQARKILFVIDGFDELGAAPGALIEDICGDWEKKKPVPVLLGSLLNRVMLPKAALLVTTRPRALRDLRILAEEPIYIRVEGFLEEDRRAYFLRHFGDEDQAMRAFELMRSNAALFQLGSAPAVCWIVCTTLKLQMEKGEDPVPTCLTRTGLFLRFLCSRFPQGAQLRGALRTLSLLAAQGLWAQTSVLHREDLERLGVQESDLRLFLDGDILRQDRVSKGCYSFIHLSFQQFLTALFYTLEKEEEEDRDGHTWDIGDVQKLLSGVERLRNPDLIQAGYYSFGLANEKRAKELEATFGCRMSPDIKQELLRCDISCKGGHSTVTDLQELLGCLYESQEEELVKEVMAQFKEISLHLNAVDVVPSSFCVKHCRNLQKMSLQVIKENLPENVTASESDAEVERSQDDQHMLPFWTDLCSIFGSNKDLMGLAINDSFLSASLVRILCEQIASDTCHLQRVVFKNISPADAHRNLCLALRGHKTVTYLTLQGNDQDDMFPALCEVLRHPECNLRYLGLVSCSATTQQWADLSLALEVNQSLTCVNLSDNELLDEGAKLLYTTLRHPKCFLQRLSLENCHLTEANCKDLAAVLVVSRELTHLCLAKNPIGNTGVKFLCEGLRYPECKLQTLVLWNCDITSDGCCDLTKLLQEKSSLLCLDLGLNHIGVKGMKFLCEALRKPLCNLRCLWLWGCSIPPFSCEDLCSALSCNQSLVTLDLGQNPLGSSGVKMLFETLTCSSGTLRTLRLKIDDFNDELNKLLEEIEEKNPQLIIDTEKHHPWAERPSSHDFMI

SEQ ID NO: 44 - protein, 353 aa, Homo sapiens
MTIFHPITSSIGQPGCGPKCKETPLELVFVIDSSESVGPENFQIIKNFVKTMADRVALDLATARIGIINYSHKVEKVANLKQFSSKDDFKLAVDNMQYLGEGTYTATALQAANDMFEDARPGVKKVALVITDGQTDSRDKEKLTEVVKNASDTNVEIFVIGVVKKNDPNFEIFHKEMNLIATDPEHVYQFDDFFTLQDTLKQKLFQKICEDFDSYLVQIFGSSSPQPGFGMSGEELSESTPEPQKEISESLSVTRDQDEDDKAPEPTWADDLPATTSSEATTTPRPLLSTPVDGAEDPRCLEALKPGNCGEYVVRWYYDKQVNSCARFWFSGCNGSGNRFNSEKECQETCIQG

SEQ ID NO: 45 - protein, 448 aa, Homo sapiens
MHEVIESDYEGRDKTLSCLVVGVCDYSTRMLGRNDHTAVTGQQGAWSESASLDHSPILSFLPQEFPADRDGSLALHSTYESLRLSASSWTVNPLRGINMMPSSLAPSSQGCGPKCKETPLELVFVIDSSESVGPENFQIIKNFVKTMADRVALDLATARIGIINYSHKVEKVANLKQFSSKDDFKLAVDNMQYLGEGTYTATALQAANDMFEDARPGVKKVALVITDGQTDSRDKEKLTEVVKNASDTNVEIFVIGVVKKNDPNFEIFHKEMNLIATDPEHVYQFDDFFTLQDTLKQKLFQKICEDFDSYLVQIFGSSSPQPGFGMSGEELSESTPEPQKEISESLSVTRDQDEDDKAPEPTWADDLPATTSSEATTTPRPLLSTPVDGAEDPRCLEALKPGNCGEYVVRWYYDKQVNSCARFWFSGCNGSGNRFNSEKECQETCIQG

SEQ ID NO: 46 - protein, 493 aa, Homo sapiens
MLPAAPSGCPQLCRCEGRLLYCEALNLTEAPHNLSGLLGLSLRYNSLSELRAGQFTGLMQLTWLYLDHNHICSVQGDAFQKLRRVKELTLSSNQITQLPNTTFRPMPNLRSVDLSYNKLQALAPDLFHGLRKLTTLHMRANAIQFVPVRIFQDCRSLKFLDIGYNQLKSLARNSFAGLFKLTELHLEHNDLVKVNFAHFPRLISLHSLCLRRNKVAIVVSSLDWVWNLEKMDLSGNEIEYMEPHVFETVPHLQSLQLDSNRLTYIEPRILNSWKSLTSITLAGNLWDCGRNVCALASWLNNFQGRYDGNLQCASPEYAQGEDVLDAVYAFHLCEDGAEPTSGHLLSAVTNRSDLGPPARRATTASRTGGEGQHDGTFKPATGGFPAGEHAKNPVQIHKVVTGTMAFIFSFLMVVLVLYVSWKCFPASLRQLRQCFVTQRRKQKQKQTMHQMAAMSAQEYYVDYKPNHIEGALVIINEYGSCTCHQQPARECEV

SEQ ID NO: 47 - protein, 548 aa, Homo sapiens
MPALRPLLPLLLLLRLTSGAGLLPGLGSHPGVCPNQLSPNLWVDAQSTCERECSRDQDCAAAEKCCINVCGLHSCVAARFPGSPAAPTTAASCEGFVCPQQGSDCDIWDGQPVCRCRDRCEKEPSFTCASDGLTYYNRCYMDAEACLRGLHLHIVPCKHVLSWPPSSPGPPETTARPTPGAAPVPPALYSSPSPQAVQVGGTASLHCDVSGRPPPAVTWEKQSHQRENLIMRPDQMYGNVVVTSIGQLVLYNARPEDAGLYTCTARNAAGLLRADFPLSVVQREPARDAAPSIPAPAECLPDVQACTGPTSPHLVLWHYDPQRGGCMTFPARGCDGAARGFETYEACQQACARGPGDACVLPAVQGPCRGWEPRWAYSPLLQQCHPFVYGGCEGNGNNFHSRESCEDACPVPRTPPCRACRLRSKLALSLCRSDFAIVGRLTEVLEEPEAAGGIARVALEDVLKDDKMGLKFLGTKYLEVTLSGMDWACPCPNMTAGDGPLVIMGEVRDGVAVLDAGSYVRAASEKRVKKILELLEKQACELLNRFQD

SEQ ID NO: 48 - protein, 286 aa, Homo sapiens
MAFVAIVVSNFGLSGQPHGGFNSQDQNDQGPSVPVSLLDRTTGGGSALCFLAGIDYKTTTILLDGRRVKLELWDTSGQGRFCTIFRSYSRGAQGILLVYDITNRWSFDGIDRWIKEIDEHAPGVPRILVGNRLHLAFKRQVPTEQARAYAEKNCMTFFEVSPLCNFNVIESFTELSRIVLMRHGMEKIWRPNRVFSLQDLCCRAIVSCTPVHLIDKLPLPVTIKSHLKSFSMANGMNAVMMHGRSYSLASGAGGGGSKGNSLKRSKSIRPPQSPPQNCSRSNCKIS

SEQ ID NO: 49 - protein, 172 aa, Homo sapiens
MGIPIPIIPHHPQARVASPQALMDKWPWKASSAAPGFCHHPSTKWSRDPGRHPESPHRGGSGVHRRSREPAPHPASEESSFPWLEDPVMKYVGKGGYNCTLSKTEFLSFMNAELAAFTKNQKDPGVLHRMMKKLGTNNDGQLDFSEFLNLIGGLAMACHDSFLKAVPSQKRT

SEQ ID NO: 50 - protein, 103 aa, Homo sapiens
LQKSPALQRLSIESLISLFQKYVGKGGYNCTLSKTEFLSFMNAELAAFTKNQKDPGVLHRMMKKLGTNNDGQLDFSEFLNLIGGLAMACHDSFLKAVPSQKRT

SEQ ID NO: 51 - protein, 753 aa, Homo sapiens
MRPVSVWQWSPWGLLLCLLCSSCLGSPSPSTGPEKKAGSQGLRFRLAGFPRKPYEGRVEIQRAGEWGTICDDDFTLQAAHILCRELGFTEATGWTHSAKYGPGTGRIWLDNLSCSGTEQSVTECASRGWGNSDCTHDEDAGVICKDQRLPGFSDSNVIEVEHHLQVEEVRIRPAVGWGRRPLPVTEGLVEVRLPDGWSQVCDKGWSAHNSHVVCGMLGFPSEKRVNAAFYRLLAQRQQHSFGLHGVACVGTEAHLSLCSLEFYRANDTARCPGGGPAVVSCVPGPVYAASSGQKKQQQSKPQGEARVRLKGGAHPGEGRVEVLKASTWGTVCDRKWDLHAASVVCRELGFGSAREALSGARMGQGMGAIHLSEVRCSGQELSLWKCPHKNITAEDCSHSQDAGVRCNLPYTGAETRIRLSGGRSQHEGRVEVQIGGPGPLRWGLICGDDWGTLEAMVACRQLGLGYANHGLQETWYWDSGNITEVVMSGVRCTGTELSLDQCAHHGTHITCKRTGTRFTAGVICSETASDLLLHSALVQETAYIEDRPLHMLYCAAEENCLASSARSANWPYGHRRLLRFSSQIHNLGRADFRPKAGRHSWVWHECHGHYHSMDIFTHYDILTPNGTKVAEGHKASFCLEDTECQEDVSKRYECANFGEQGITVGCWDLYRHDIDCQWIDITDVKPGNYILQVVINPNFEVAESDFTNNAMKCNCKYDGHRIWVHNCHIGDAFSEEANRRFERYPGQTSNQII

SEQ ID NO: 52 - protein, 114 aa, Homo sapiens [sequence omitted]

SEQ ID NO: 53 - protein, 106 aa, Homo sapiens
MAKISGCTEIAWWCITTLCCFPERYAGRDHNSCKLSQRGFLNFMNTVLVAFTKNQKGSGALDCMMKKLDFNCDGQLDFQDFLSLTDGVAVACPDSFIPAGHAHERI

SEQ ID NO: 54 - protein, 643 aa, Homo sapiens
MALAGPCPSSTASLLPSTQALPTINSFLKIASKPKSTLDRAVGKASSILALKSRASAKRSVLLPILALWAGSCSGGAPPTPMGLATLQLLPSPPGAPDGQLQPIPGIGHPDKPEAGKLDQLRDQPTPKQGAQGTPTQSPSTGWKALPRPGLALRKESPPVTLEQEQGHNKGLVAEWAQPQATAAMRAGAGKPEALKLRPWQAGRDPQAQEGAAVTEEDQGQRTGGREDKGRGLKPRRPPKGTSHQPGLRIRRPQKDRSRGQGGGGSTSKTPGHGWKRPGSTHGHRHRHADLGTTQQAMPSLPASCLLAQAVIACGNVKMKHVPALTHPGLTTLYLAENEIAKIPAHTFLGLPNLEWLDLSKNKLDPRGLHPHAFKNLMRLKRLNLVGNSLTTVPALPASLQELKLNDNLLQGLQGSSFRGLSQLLTLEELHLGTNLIEEVAEGALSHIHSLSVLVLSHNWLQEHWLAPRAWIHLPKLETLDLSYNRLVHVPRFLPRGLRRLTLHHDHIERIPGYAFAHMKPGLEFLHLSHNRLQADGIHSVSFLGLRASLAELLLDHNQVQAIPRGLLGLKGLQVLGLSHNRIRQVPLNSICDMRVAQDSNLTSTHLENNLIDRRRIPPTAFSCTRAYHSVVLQPQRRGEEGS

SEQ ID NO: 55 - protein, 653 aa, Homo sapiens [sequence omitted]

SEQ ID NO: 56 - protein, 305 aa, Homo sapiens
MGARGALLLALLLARAGLGKPESQEEELLSEACGHREIHALVAGGVESARGRWPWQASLRLRRRHRCGGSLLSRRWVLSAAHCFQKHYYPSEWTVQLGELTSRPTPWNLRAYSSRYKVQDIIVNPDALGVLRNDIALLRLASSVTYNAYIQPICIESSTFNFVHRPDCWVTGWGLISPSGTPLPPPYNLREAQVTILNNTRCNYLFEQPSSRSMIWDSMFCAGAEDGSVDTCKGDSGGPLVCDKDGLWYQVGIVSWGMDCGQPNRPGVYTNISVYFHWIRRVMSHSTPRPNPSQLLLLLALLWAP

SEQ ID NO: 57 - protein, 387 aa, Homo sapiens
MRVTWNHGPPCPSPDSLTITCNYGNGGCQHSCEDTDTGPTCGCHQKYALHSDGRTCIEKDEAAIERSQFNATSVADVDKRVKRRLLMAPPDWGQKLGLFQLGAPPQGTAQGLAQSGSMESLLINLVIEHNSLDTSAVLVTLTLPCPDSVWSVGEASAHTDSAALWGRSPGVSALPTSWRRKPGHQRVQTSRPRRLSRPPQVCFRVGEIPHEAIMSAPETCAVNNGGCDRTCKDTATGVRCSCPVGFTLQPDGKTCKDINECLVNNGGCDHFCRNTVGSFECGCRKGYKLLTDERTCQDIDECSFERTCDHICINSPGSFQCLCHRGYILYGTTHCGDVDECSMSNGSCDQGCVNTKGSYECVCPPGRRLHWNGKDCVGRGSLLLGYG

SEQ ID NO: 58 - protein, 964 aa, Homo sapiens
MGAAAVRWHLCVLLALGTRGRLAGGSGLPGSVDVDECSEGTDDCHIDAICQNTPKSYKCLCKPGYKGEGKQCEDIDECENDYYNGGCVHECINIPGNYRCTCFDGFMLAHDGHNCLDVDECQDNNGGCQQICVNAMGSYECQCHSGFFLSDNQHTCIHRSNEGMNCMNKDHGCAHICRETPKGGVACDCRPGFDLAQNQKDCTLTCNYGNGGCQHSCEDTDTGPTCGCHQKYALHSDGRTCIETCAVNNGGCDRTCKDTATGVRCSCPVGFTLQPDGKTCKDINECLVNNGGCDHFCRNTVGSFECGCRKGYKLLTDERTCQDIDECSFERTCDHICINSPGSFQCLCHRGYILYGTTHCGDVDECSMSNGSCDQGCVNTKGSYECVCPPGRRLHWNGKDCVETGKCLSRAKTSPRAQLSCSKAGGVESCFLSCPAHTLFVPDSENSYVLSCGVPGPQGKALQKRNGTSSGLGPSCSDAPTTPIKQKARFKIRDAKCHLRPHSQARAKETARQPLLDHCHVTFVTLKCDSSKKRRRGRKSPSKEVSHITAEFEIETKMEEASDTCEADCLRKRAEQSLQAAIKTLRKSIGRQQFYVQVSGTEYEVAQRPAKALEGQGACGAGQVLQDSKCVACGPGTHFGGELGQCVSCMPGTYQDMEGQLSCTPCPSSDGLGLPGARNVSECGGQCSPGFFSADGFKPCQACPVGTYQPEPGRTGCFPCGGGLLTKHEGTTSFQDCEAKVHCSPGHHYNTTTHRCIRCPVGTYQPEFGQNHCITCPGNTSTDFDGSTNVTHCKNQHCGGELGDYTGYIESPNYPGDYPANAECVWHIAPPPKRRILIVVPEIFLPIEDECGDVLVMRKSASPTSITTYETCQTYERPIAFTSRSRKLWIQFKSNEGNSGKGFQVPYVTYDGKIHCLHGPLCTAQAGPWRHRDESHVPALRELRPGRYRPGSRTNTVRGQSQTG

SEQ ID NO: 59 - protein, 213 aa, Homo sapiens
AMVLPSYSKSEGGSLLDIYCLLTYWMEVVPTLLAETKIPATDVADASLNECSSTERKQDVVLLFVTLSHTQPPLFHLPYVQKPLISNVEQLILGIPGQNRREIGHGQDIFPAEKLCHLQDRKVNLHRAAWGECIVAPKTLSFSYCQGTCPALNSELRHSSFECYKRAVPTCPWLFQTCRPTMVRLFSLMVQDDEHKMSVHYVNTSLVEKCGCS

SEQ ID NO: 60 - protein, 189 aa, Homo sapiens
BMEVVPTLLAETKIPATDVADASLNECSSTERKQDVVLLFVTLSHTQPPLFHLPYVQKPLISNVEQLILGIPGQNRREIGHGQDIFPAEKLCHLQDRKVNLHRAAWGECIVAPKTLSFSYCQGTCPALNSELRHSSFECYKRAVPTCPWLFQTCRPTMVRLFSLMVQDDEHKMSVHYVNTSLVEKCGCS

SEQ ID NO: 61 - protein, 740 aa, Homo sapiens
MGDSGAEAVGGGGTYTDGPVLLLYAGELLLPQETTVELSCGVGPLQVILGPEQAAVLNCSLGAAAAGPPTRVTWSKDGDTLLEHDHLHLLPNGSLWLSQPLAPNGSDESVPEAVGVIEGNYSCLAHGPPGVLASQTAVVKLATLADFSLHPESQTVEENGTARFECHIEGLPAPIITWEKDQVTLPEEPRLIVLPNGVLQILDVQESDAGPYRCVATNSARQHFSQEALLSVAHRGSLASTRGQDVVIVAAPENTTVVSGQSVVMECVASADPTPFVSWVRQDGKPISTDVIVLGRTNLLIANAQPWHSGVYVCRANKPRTRDFATAAAELRVLAAPAITQAPEALSRTRASTARFVCRASGEPRPALRWLHNGAPLRPNGRVKVQGGGGSLVITQIGLQDAGYYQCVAENSAGMACAAASLAVVVREGLPSAPTRVTATPLSSSAVLVAWERPEMHSEQIIGFSLHYQKARGMDNVEYQFAVNNDTTELQVRDLEPNTDYEFYVVAYSQLGASRTSTPALVHTLDDGRASELAVGSLGLSNGQVVKYKIEYGLGKEDQIFSTEVRGNETQLMLNSLQPNKVYRVRISAGTAAGFGAPSQWMHHRTPSMHNQSHVPFAPAELKVQAKMESLVVSWQPPPHPTQISGYKLYWREVGAEEEANGDRLPGGRGDQAWDVGPVRLKKKVKQYELTQLVPGRLYEVKLVAFNKHEDGYAAVWKGKTEKAPAPGEGGGGRRRGGLR

SEQ ID NO: 62 - protein, 1250 aa, Homo sapiens
MARGDAGRGRGLLALTFCLLAARGELLLPQETTVELSCGVGPLQVILGPEQAAVLNCSLGAAAAGPPTRVTWSKDGDTLLEHDHLHLLPNGSLWLSQPLAPNGSDESVPEAVGVIEGNYSCLAHGPLGVLASQTAVVKLATLADFSLHPESQTVEENGTARFECHIEGLPAPIITWEKDQVTLPEEPRLIVLPNGVLQILDVQESDAGPYRCVATNSARQHFSQEALLSVAHRGSLASTRGQDVVIVAAPENTTVVSGQSVVMECVASADPTPFVSWVRQDGKPISTDVIVLGRTNLLIANAQPWHSGVYVCRANKPRTRDFATAAAELRVLAAPAITQAPEALSRTRASTARFVCRASGEPRPALRWLHNGAPLRPNGRVKVQGGGGSLVITQIGLQDAGYYQCVAENSAGMACAAASLAVVVREGLPSAPTRVTATPLSSSAVLVAWERPEMHSEQIIGFSLHYQKARGMDNVEYQFAVNNDTTELQVRDLEPNTDYEFYVVAYSQLGASRTSTPALVHTLDDVPSAAPQLSLSSPNPSDIRVAWLPLPPSLSNGQVVKYKIEYGLGKEDQIFSTEVRGNETQLMLNSLQPNKVYRVRISAGTAAGFGAPSQWMHHRTPSMHNQSHVPFAPAELKVQAKMESLVVSWQPPPHPTQISGYKLYWREVGAEEEANGDRLPGGRGDQAWDVGPVRLKKKVKQYELTQLVPGRLYEVKLVAFNKHEDGYAAVWKGKTEKAPAPDMPIQRGPPLPPAHVHAESNSSTSIWLRWKKPDFTTVKIVNYTVRFSPWGLRNASLVTYYTSSGEDILIGGLKPFTKYEFAVQSHGVDMDGPFGSVVERSTLPDRPSTPPSDLRLSPLTPSTVRLHWCPPTEPNGEIVEYLILYSSNHTQPEHQWTLLTTQGNIFSAEVHGLESDTRYFFKMGARTEVGPGPFSRLQDVITLQEKLSDSLDMHSVTGIIVGVCLGLLCLLACMCAGLRRSPHRESLPGLSSTATPGNPALYSRARLGPPSPPAAHELESLVHPHPQDWSPPPSDVEDRAEVHSLMGGGVSEGRSHSKRKISWAQPSGLSWAGSWAGCELPQAGPRPALTRALLPPAGTGQTLLLQALVYDAIKGNGRKKSPPACRNQVEAEVIVHSDFSASNGNPDLHLQDLEPEDPLPPEAPDLISGVGDPGQGAAWLDRELGGCELAAPGPDRLTCLPEAASASCSYPDLQPGEVLEETPGDSCQLKSPCPLGASPGLPRSPVSSSA

SEQ ID NO: 63 - protein, 634 aa, Homo sapiens
MAQGVLWILLGLLLWSDPGTASLPLLMDSVIQALAELEQKVPAAKTRHTASAWLMSAPNSGPHNRLYHFLLGAWSLNATELDPCPLSPELLGLTKEVARHDVREGKEYGVVLAPDGSTVAVEPLLAGLEAGLQGRRVINLPLDSMAAPWETGDTFPDVVAIAPDVRATSSPGLRDGSPDVTTADIGANTPDATKGCPDVQASLPDAKAKSPPTMVDSLLAVTLAGNLGLTFLRGSQTQSHPDLGTEGCWDQLSAPRTFTLLDPKASLLTMAFLNGALDGVILGDYLSRTPEPRPSLSHLLSQYYGAGVARDPGFRSNFRRQNGAALTSASILAQQVWGTLVLLQRLEPVHLQLQCMSQEQLAQVAANATKEFTEAFLGCPAIHPRCRWGAAPYRGRPKLLQLPLGFLYVHHTYVPAPPCTDFTRCAANMRSMQRYHQDTQGWGDIGYSFVVGSDGYVYEGRGWHWVGAHTLGHNSRGFGVAIVGNYTAALPTEAALRTVRDTLPSCAVRAGLLRPDYALLGHRQLVRTDCPGDALFDLLRTWPHFTAVSLRSLHYTARRPSVYTSSTRPLPPACNSCARTASARPPTSRRHVYSGNLGPAFAGHSAGNIPDPVTSAYAASAQPQTQPACPFPSS

SEQ ID NO: 64 - protein, 576 aa, Homo sapiens
MAQGVLWILLGLLLWSDPGTASLPLLMDSVIQALAELEQKVPAAKTRHTASAWLMSAPNSGPHNRLYHFLLGAWSLNATELDPCPLSPELLGLTKEVARHDVREGKEYGVVLAPDGSTVAVEPLLAGLEAGLQGRRVINLPLDSMAAPWETGDTFPDVVAIAPDVRATSSPGLRDGSPDVTTADIGANTPDATKGCPDVQASLPDAKAKSPPTMVDSLLAVTLAGNLGLTFLRGSQTQSHPDLGTEGCWDQLSAPRTFTLLDPKASLLTMAFLNGALDGVILGDYLSRTPEPRPSLSHLLSQYYGAGVARDPGFRSNFRRQNGAALTSASILAQQVWGTLVLLQRLEPVHLQLQCMSQEQLAQVAANATKEFTEAFLGCPAIHPRCRWGAAPYRGRPKLLQLPLGFLYVHHTYVPAPPCTDFTRCAANMRSMQRYHQDTQGWGDIGYSFVVGSDGYVYEGRGWHWVGAHTLGHNSRGFGVAIVGNYTAALPTEAALRTVRDTLPSCAVRAGLLRPDYALLGHRQLVRTDCPGDALFDLLRTWPHFTATVKPRPARSVSKRSRREPPPRTLPATDLQ

SEQ ID NO: 65 - protein, 734 aa, Homo sapiens
MWGLLLALAAFAPAVGPALGAPRNSVLGLAQPGTTKVPGSTPALHSSPAQPPAETANGTSEQHVRIRVIKKKKVIMKKRKKLTLTRPTPLVTAGPLVTPTPAGTLDPAEKQETGCPPLGLESLRVSDSRLEASSSQSFGLGPHRGRLNIQSGLEDGDLYDGAWCAEEQDADPWFQVDAGHPTRFSGVITQGRNSVWRYDWVTSYKVQFSNDSRTWWGSRNHSSGMDAVFPANSDPETPVLNLLPEPQVARFIRLLPQTWLQGGAPCLRAEILACPVSDPNDLFLEAPASGSSDPLDFQHHNYKAMRKLMKQVQEQCPNITRIYSIGKSYQGLKLYVMEMSDKPGEHELGEPEVRYVAGMHGNEALGRELLLLLMQFLCHEFLRGNPRVTRLLSEMRIHLLPSMNPDGYEIAYHRGSELVGWAEGRWNNQSIDLNHNFADLNTPLWEAQDDGKVPHIVPNHHLPLPTYYTLPNATVAPETRAVIKWMKRIPFVLSANLHGGELVVSYPFDMTRTPWAARELTPTPDDAVFRWLSTVYAGSNLAMQDTSRRPCHSQDFSVHGNIINGADWHTVPGSMNDFSYLHTNCFEVTVELSCDKFPHENELPQEWENNKDALLTYLEQVRMGIAGVVRDKDTELGIADAVIAVDGINHDVTTAWGGDYWRLLTPGDYMVTASAEGYHSVTRNCRVTFEEGPFPCNFVLTKTPKQRLRELLAAGAKVPPDLRRRLERLRGQKD

SEQ ID NO: 66 - protein, 358 aa, Homo sapiens
MPEDVREKKENLLLNSERSTRLLTKTSHSQGGDQALSKSTGSPTEKLIEKRQGAKTVFNKFSNMNWPVDIHPLNKSLVKDNKWKKTEETQEKRRSFLQEFCKKYGGVSHHQSHLFHTVSRIYVEDKHKILYCEVPKAGCSNWKRILMVLNGLASSAYNISHNAVHYGKHLKKLDSFDLKGIYTRLNTYTKAVFVRDPMERLVSAFRDKFEHPNSYYHPVFGKAIIKKYRPNACEEALINGSGVKFKEFIHYLLDSHRPVGMDIHWEKVSKLCYPCLINYDFVGKFETLEEDANYFLQMIGAPKELKFPNFKDRHSSDERTNAQVVRQYLKDLTRTERQLIYDFYYLDYLMFNYTTPFL